SEQUENCE LISTING (47 sequences)

SEQ ID NO: 1 - DNA, 797 bp, Solanum tuberosum
  CDS (3)..(797)
  SITE 214: Xaa = Ala or Thr
gatccaatgcggtgaatcggatgattgagagctctatgaagggtgtagagttttggattgtgaacactgatatccaagcaatgaggatgtcacctgtaaatcctgagcatagactgccaataggtcaagaactcacaaggggacttggcgcaggcggtaatccggatatagggatgaatgctgccaatgagagtaagcaggccattgaggaagcagtttacggctcagacatggtttttgtgactgctggaatgggtggaggaacagggactggtgcggctcctataattgcaggaactgccaaatcaatgggtatcttaactgttggtatcgttacaacccccttttcttttgagggacgaagaagagcagttcaagcccaagaaggaattgcagctttgagagaaaatgttgatactctgattgtcattccaaatgacaaattattgacagctgtttctccatcaaccccagtaactgaagcttttaacctggctgatgatattcttcggcaaggagttcgtggtatttctgatataattacgattcctgggctagttaatgtggattttgctgatgtgcgtgctattatggcaaatgctgggtcttctttaatgggrataggractgcyacgggraagrccagagcaagagatgcagcattgaacgccattcaatcgcctctgctggacattggtatagagagggctactggaattgtgtggaatataactggtggaagtgatctaacattatttgaggtaaatgctgcagcagaggttatatatgaccttgtg

SEQ ID NO: 2 - protein, 265 aa, Solanum tuberosum
  SITE 214: Xaa = Ala or Thr
SNAVNRMIESSMKGVEFWIVNTDIQAMRMSPVNPEHRLPIGQELTRGLGAGGNPDIGMNAANESKQAIEEAVYGSDMVFVTAGMGGGTGTGAAPIIAGTAKSMGILTVGIVTTPFSFEGRRRAVQAQEGIAALRENVDTLIVIPNDKLLTAVSPSTPVTEAFNLADDILRQGVRGISDIITIPGLVNVDFADVRAIMANAGSSLMGIGTATGKXRARDAALNAIQSPLLDIGIERATGIVWNITGGSDLTLFEVNAAAEVIYDLV

SEQ ID NO: 3 - DNA, 833 bp, Solanum tuberosum
  CDS (1)..(831)
  SITE 7: Xaa = Arg or Ser
ggatccaaygcdgtdaatmgbatgattgagagctctatgaatggtgtggagttttggattgtgaatactgatattcaggcaattaggatgtcacctgtgtttcctgagaatcgattgccaataggccaagagctcacgagaggactaggtgcaggtggtaatccagatatagggatgaatgctgccaaagaaagcaaggaggctattgaagaagcagttcscggtgcagatatggtttttgtgactgctggaatgggcggaggaacagggactggtggggctcctataattgcaggaattgccaaatcaatgggtatcttaactgttggtattgtcacaacccccttttcttttgagggacgaagaagagcagttcaagcccaagaaggaattgcagctttgagagaaaatgttgatacgctaattgtcattcctaatgacaagttactgactkctgtttccttatcaaccccagtaactgaagcttttaacctggctgatgatattcttcggcaaggggttcgtggtatttctgatataattacgattcctggactggtaaatgtggattttgctgatgtgcgtgctattatggcaaatgctggttcctcattgatgggaataggaactgctacagggaagaccagagccagagatgctgcattgaatgctgttcaatctcctttgctggacattggcatagagagagctactggaattgtgtggaatataaccggtggkaacgrtttaacattatttgaggtaaatgctgcagcagaggttatatatgaccttgtcgatccwagtgccaacctmatmttyggcgcggatcc

SEQ ID NO: 4 - protein, 277 aa, Solanum tuberosum
  SITE 7: Xaa = Arg or Ser
GSNAVNXMIESSMNGVEFWIVNTDIQAIRMSPVFPENRLPIGQELTRGLGAGGNPDIGMNAAKESKEAIEEAVXGADMVFVTAGMGGGTGTGGAPIIAGIAKSMGILTVGIVTTPFSFEGRRRAVQAQEGIAALRENVDTLIVIPNDKLLTXVSLSTPVTEAFNLADDILRQGVRGISDIITIPGLVNVDFADVRAIMANAGSSLMGIGTATGKTRARDAALNAVQSPLLDIGIERATGIVWNITGGNXLTLFEVNAAAEVIYDLVDPSANLIFGAD

SEQ ID NO: 5 - DNA, 827 bp, Triticum aestivum
  CDS (3)..(827)
gatccaacgctgtcaatagaatgattgagtactccatgaatggtgtcgagttttggatcgtcaacaccgatgtccaggcgataaggatgtccccggtgcatccccagaacaggctgcagattgggcaggagctcactcggggtttgggtgcgggtgggaaccctgatattgggatgaatgccgccaaggagagctgtgagtccatagaggaagcacttcatggtgctgacatggtttttgtcacggctggaatgggtggaggaactggaactggaggtgctcctgtaattgctggaattgccaagtccatgggtatactgacagtgggtattgtcacaacgcccttttcatttgagggggggaggcgtgcagttcaggctcaagaaggaatatcagccttgagaaatagtgtggacactctcattgtcatcccaaatgacaagctgttgtctgctgtttctccaaacactcctgtcacggaagcattcaacttggctgatgatattctttggcaaggaattcgcggtatctctgatatcattacggttcctgggttggttaatgtagattttgcagatgtgcgagccataatgcaaaatgcagggtcatctttgatgggtatagggactgcaacaggcaagtcaagagcaagagacgccgctcttaatgccattcagtcaccactgctagatattggaattgagagggctacaggcatcgtgtggaatatcactggaggaaatgatttgactttgtttgaggtaaatgctgcagccgaagtaatctacgatctagttgatccaaatgctaatctcatctttggcgcg

SEQ ID NO: 6 - protein, 275 aa, Triticum aestivum
SNAVNRMIEYSMNGVEFWIVNTDVQAIRMSPVHPQNRLQIGQELTRGLGAGGNPDIGMNAAKESCESIEEALHGADMVFVTAGMGGGTGTGGAPVIAGIAKSMGILTVGIVTTPFSFEGGRRAVQAQEGISALRNSVDTLIVIPNDKLLSAVSPNTPVTEAFNLADDILWQGIRGISDIITVPGLVNVDFADVRAIMQNAGSSLMGIGTATGKSRARDAALNAIQSPLLDIGIERATGIVWNITGGNDLTLFEVNAAAEVIYDLVDPNANLIFGA

SEQ ID NO: 7 - DNA, 773 bp, Triticum aestivum
  CDS (2)..(772)
  SITE 94: Xaa = Val or Asp
agctggatccggggtggagttctggattgttaataccgatgtccaggcgataaggatgtccccggtgcattcccagaacaggctgcagattgggcaggagctcactcggggtctgggtgcgggtgggaaccctgatattgggatgaatgctgctaaggagagctgtgagtccatagaggaagcacttcatggtgctgacatggtttttgtcacggcaggaatgggtgggggaactggaactggaggtgcccctgtaattgctggaattgccaagtccatggrtatactgacagtgggtattgtcacaacgcccttttcatttgaggggaggaggcgggcagttcaggctcaagaaggaacatcagccttgagaaatagtgtggacactctcattgtcatcccaaatgacaagctgttgtctgctgtttctccaaacactcctgtcacggaagcattcaacttggctgatgatattctttggcaaggaattcgcggtatctctgatatcattacggttcctgggctggttaatgttgattttgctgatgtgsgagccataatgcaaaatgcagggtcatcttygatgggtatagggactgcaacaggcaagtcaagagcaagagatgccgctcttaatgccattcagtcaccactgctagatattggaattgagagggctacaggcatcgtgtggaatatcactggaggaaatgatttgactttgtttgaggtaaatgcmgcagcagccgaagtaatmtatgatcctagggcta

SEQ ID NO: 8 - protein, 257 aa, Triticum aestivum
  SITE 94: Xaa = Val or Asp
AGSGVEFWIVNTDVQAIRMSPVHSQNRLQIGQELTRGLGAGGNPDIGMNAAKESCESIEEALHGADMVFVTAGMGGGTGTGGAPVIAGIAKSMXILTVGIVTTPFSFEGRRRAVQAQEGTSALRNSVDTLIVIPNDKLLSAVSPNTPVTEAFNLADDILWQGIRGISDIITVPGLVNVDFADVXAIMQNAGSSXMGIGTATGKSRARDAALNAIQSPLLDIGIERATGIVWNITGGNDLTLFEVNAAAAEVIYDPRA

SEQ ID NO: 9 - DNA, 795 bp, Solanum tuberosum
  CDS (10)..(795)
tagcggatccgtggcagtggcttgcagggtgttgacttctatgctataaacacggatgctcaagcactggtacagtctgctgccgagaacccacttcaaattggagaacttctgactcgtgggcttggtactggtggcaatcctcttttaggggaacaggcagcggaggagtcaaaggaagctattgcaaattctctaaaaggttcagatacggttttcataacagcaggaatgggtggaggtacaggatctggtgcggctcctgttgtggctcaaatagcaaaagaagcaggttatttgactgttggtgttgttacatatccattcagctttgaaggacgtaaaagatctgtgcaggctctggaagcaattgaaaaacttcagagaaatgttgacactcttatagtaattcccaatgatcgtctactagatattgccgatgagcagacaccacttcaagatgctttccttcttgcagatgatgtattacgtcaaggtgtccaaggaatatctgatataatcactattcctgggcttgtgaatgtggattttgccgatgtaaaggcagtgatgaaagactctggaactgctatgctcggagtgggggtttcatcaagcaagaaccgagctgaagaagcagccgaacaagcaactctggcccctctaattgggtcgtcaattcaatctgcaactggggtagtatataacattacaggaggaaaagacataactttgcaagaagcgaatagggtgtcccaggttgtcaccagcctggctgatccatggatcccagta

SEQ ID NO: 10 - protein, 262 aa, Solanum tuberosum
RGSGLQGVDFYAINTDAQALVQSAAENPLQIGELLTRGLGTGGNPLLGEQAAEESKEAIANSLKGSDTVFITAGMGGGTGSGAAPVVAQIAKEAGYLTVGVVTYPFSFEGRKRSVQALEAIEKLQRNVDTLIVIPNDRLLDIADEQTPLQDAFLLADDVLRQGVQGISDIITIPGLVNVDFADVKAVMKDSGTAMLGVGVSSSKNRAEEAAEQATLAPLIGSSIQSATGVVYNITGGKDITLQEANRVSQVVTSLADPWIPV

SEQ ID NO: 11 - DNA, 1260 bp, Solanum tuberosum
  CDS (6)..(1259)
gatccatggccaccatctcaaacccagcagagttagcttcttgtccttcttcttccttaactttttcccacaggctacatacttccttcattcctaaacaatgcttcttcaccggagttccccggaaaagtttttgccggcctcaacgtttcagcatttcaagttcatttactccgatggattctgctaagattaaggtcgtcggcgtcggtggaggtggaaacaatgccgttaaccgtatgattggtagtggcttacagggtgttgacttctatgctataaacacggatgctcaagcactggtacagtctgctgccgagaacccacttcaaattggagaacttctgactcgtgggcttggtactggtggcaatcctcttttaggggaacaggcagcggaggagtcaaaggaagctattgcaaattctctaaaaggttcagatatggttttcataacagcaggaatgggtggaggtacaggatctggtgcggctcctgttgtggctcaaatagcaaaagaagcaggttatttgactgttggtgttgttacatatccgttcagctttgaaggacgtaaaagatctgtgcaggctctggaagcaattgaaaaacttcagagaaatgttgacactcttatagtaattcccaatgatcgtctgctagatattgccgatgagcagacaccacttcaagatgctttccttcttgcagatgatgtattacgtcaaggtgtccaaggaatatctgatataatcactattcctgggcttgtgaatgtggattttgccgatgtaaaggcagtgatgaaagactctggaactgctatgctcggagtgggggtttcatcaagcaagaaccgagctgaagaagcagccgaacaagcaactctggcccctctaattgggtcgtcaattcaatctgcaactggggtagtatataacattacaggaggaaaagacataactttgcaagaagtgaatagggtgtcccaggttgttaccagtctggctgatccctctgctaacatcatatttggtgctgttgttgatgagcgttacaatggtgaaatacacgtgacaataattgcaactggtttcacccagtcgtttcagaagacacttctatctgacccacgaggagcaaagctacttgagaagggctctggaatcaaagaaagcatggcatcacctgttaccctgagatcatcaaactcaccttcaacaacctcacggacacctactcggaggctgttcttttagg

SEQ ID NO: 12 - protein, 417 aa, Solanum tuberosum
MATISNPAELASCPSSSLTFSHRLHTSFIPKQCFFTGVPRKSFCRPQRFSISSSFTPMDSAKIKVVGVGGGGNNAVNRMIGSGLQGVDFYAINTDAQALVQSAAENPLQIGELLTRGLGTGGNPLLGEQAAEESKEAIANSLKGSDMVFITAGMGGGTGSGAAPVVAQIAKEAGYLTVGVVTYPFSFEGRKRSVQALEAIEKLQRNVDTLIVIPNDRLLDIADEQTPLQDAFLLADDVLRQGVQGISDIITIPGLVNVDFADVKAVMKDSGTAMLGVGVSSSKNRAEEAAEQATLAPLIGSSIQSATGVVYNITGGKDITLQEVNRVSQVVTSLADPSANIIFGAVVDERYNGEIHVTIIATGFTQSFQKTLLSDPRGAKLLEKGSGIKESMASPVTLRSSNSPSTTSRTPTRRLFF

SEQ ID NO: 13 - DNA, 1434 bp, Solanum tuberosum
  CDS (1)..(1434)
  SITE 13: Xaa = Asp or Gly
atggctacttgtacatcagctgtgtttatgcctcctgrtacgcgacggtcacgaggggtattgactgttcttggtggtagagtttgccctttgaaaattcaagatgaraagattggatatctgggcgttaaccaaaagggtacctcaagtttgcctcaattcaaatgttcagccaattcccacagtgtcaatcagtatcaaaacaaagacccctttctcaatctacatcccgaaatttctatgctcagaggcgaaggtaacaatacaatgactacctctagacaagaaagctcaagtggaaatgtcagtgagagtttgatggattcatcaagctcgaacaattttaatgaggccaaaatcaaggtggttggtgtaggaggtggtggatcaaatgcagttaatcgcatgattgagagctctatgaagggtgtagagttttggattgtgaacactgatatccaagcaatgaggatgtcacctgtaaatcctgagcatagactgccaataggtcaagaactcacaaggggacttggcgcaggcggtaatccagatatagggatgaatgctgccaatgagagtaagcaggccattgagggagcagtttacggctcagacatggtttttgtgactgctggaatgggtggagggacagggacttgtgcggctcctataattkcaggaactkcyaaatcaatgggtatcttactgttggtattgttacaacccccttttctttcgaggggacgaagamgagcagttcaagccmaagaaggwattgcagctttgagagaaaatgtygatactctaattgtcattccaaatgacaaattattgacagctgtttctccatcaacccaagtaactgaagcttttaacctggctgatgatattcttcggcaaggagttcgtggtatttctgatataattacgattcctgggctagtaaatgtggattttgctgatgtgcgtgctattatggcaaatgctgggtcttctttaatgggaataggaactgctacgggaaagaccagagcaagagatgcagcattgaacgccattcaatctcctctgctggacattggtatagagagggctactggaattgtgtggaatataactggtggtagtgatctaacattatttgaggtaaatgctgcagcagaggttatatatgaccttgtggatccaagtgctaacctcatttttggggcggtgatagacccatcgataagtggacaggtcagcataacgctaattgccackggtttcaaacgccaagaagaaagtgatatgaggtccactaccagggagatgcttcacttggaactaacagacgacctgcgtcctttttggaaggtggttcagtggaaattcctgagttcctcagaaaaaaaggacgatcacgctacccaacagcttaagaagatccccggg

SEQ ID NO: 14 - protein, 478 aa, Solanum tuberosum
  SITE 13: Xaa = Asp or Gly
MATCTSAVFMPPXTRRSRGVLTVLGGRVCPLKIQDEKIGYLGVNQKGTSSLPQFKCSANSHSVNQYQNKDPFLNLHPEISMLRGEGNNTMTTSRQESSSGNVSESLMDSSSSNNFNEAKIKVVGVGGGGSNAVNRMIESSMKGVEFWIVNTDIQAMRMSPVNPEHRLPIGQELTRGLGAGGNPDIGMNAANESKQAIEGAVYGSDMVFVTAGMGGGTGTCAAPIIXGTXKSMGILLLVLLQPPFLSRGRRRAVQAXEGIAALRENVDTLIVIPNDKLLTAVSPSTQVTEAFNLADDILRQGVRGISDIITIPGLVNVDFADVRAIMANAGSSLMGIGTATGKTRARDAALNAIQSPLLDIGIERATGIVWNITGGSDLTLFEVNAAAEVIYDLVDPSANLIFGAVIDPSISGQVSITLIATGFKRQEESDMRSTTREMLHLELTDDLRPFWKVVQWKFLSSSEKKDDHATQQLKKIPG

SEQ ID NO: 15 - DNA, 446 bp, Triticum aestivum
  CDS (3)..(446)
  SITE 143: Xaa = Gly, Arg or STOP
tggaccttcacccggaggtgtccctgctccgaggcgagcagaatgacgaggctattaacccaaggaaagcttcttctgatgggagcacgttggaggggctgggggtgccgccgagccaggacgattacaacgctgccaagatcaaggtcgtcggagtcgggggtgggggttcgaatgctgtcaacaggatgattgagtactccatgaatggtgtcgagttttggatcgtcaacaccgatgtccaggcgataaggatgtccccggtgcattcccagaacaggctgcagattgggcaggagctcactcggggtttgggtgcgggtgggaaccctgatattgggatgaatgccgccaaggagagctgtgagtccatagaggaagcacttcatggtgctgacatggtttttgtcaccgctggaatgggtggangaactggaactggangn

SEQ ID NO: 16 - protein, 148 aa, Triticum aestivum
  SITE 143: Xaa = Gly, Arg or STOP
DLHPEVSLLRGEQNDEAINPRKASSDGSTLEGLGVPPSQDDYNAAKIKVVGVGGGGSNAVNRMIEYSMNGVEFWIVNTDVQAIRMSPVHSQNRLQIGQELTRGLGAGGNPDIGMNAAKESCESIEEALHGADMVFVTAGMGGXTGTGX

SEQ ID NO: 17 - DNA, 640 bp, Zea mays
  CDS (1)..(336)
gaggcagctactggcgttgtgtataatattactggtgggaaggacatcactttgcaagaagtgaacaaggtgtcccagattgtgacaagcctagctgacccatctgcgaacataattttcggtgctgtcgttgatgaccgttacactggtgagatacatgtgacaatcattgcgacaggatttccacagtccttccagaaatcccttttggcagatccaaagggagcacgaatagtggaatccaaagagaaagcagcaaccctcgcccataaagcagcagctgctgcagttcaaccggtccctgcttctgcttggtctcgaagactcttctcctgagaagctcatttggtgaaccgtgactcgtagtgcattagatttgcatttagcgtgttgagggcagtccctaaggtgatcttcggatatctggagatttatagcttgggctagtgttcggtagtggtagaataagtttcagtgtatgtatcgttgctttgctttatgtttttgaggatcaggcggtgaggctgagagaagtgctcagcaactcaacattgaactgttgtagaagatctttgattgcttttattgctgctacatgccaacatccctctgttggattcagcaagggggaaaaaaaaaa

SEQ ID NO: 18 - protein, 111 aa, Zea mays
EAATGVVYNITGGKDITLQEVNKVSQIVTSLADPSANIIFGAVVDDRYTGEIHVTIIATGFPQSFQKSLLADPKGARIVESKEKAATLAHKAAAAAVQPVPASAWSRRLFS

SEQ ID NO: 19 - DNA, 833 bp, Oryza sativa
  CDS (1)..(516)
ggaatatcagatattattacaatacctggacttgtcaatgttgattttgctgatgtgaaagctgttatgaaaaactctggaactgcaatgcttggtgttggtgtttcttccagcaaaaatcgggcccaagaagctgcaagacaggcaacacttgctcctttaatcgggtcgtctattgaggcggctactggtgttgtgtacaatatcactggtggaaaggacataaccttgcaagaagtaaacaaagtctctcagattgtgacaagcttggccgatccttctgcaaatataattttcggggctgttgttgatgaccggtacactggtgagattcatgtgacgatcattgccacagggtttccacaatcctttcagaagtcccttttggccgatcccaagggtgcaagaataatggaggccaaagaaaaggcagcgaacctcacctataaagcagtggcagcggcgacggtacaaccagcgcccgccgccacttggtctcggaggctcttttcctgaacacggttcaataggaaaactagtagtttgtgtaccttagattctcatggaattactgagttggcgctccaatcaggcttctatgtgttattctttttggatatgtaaacacttaacagttacacatagtgattagcttcacttttattgtatgtatcatctagatgaggttgaggtcttcaggagttcagcagccgtcacgaatttttattgtatgttcaagacgacacttggtagttgttcgttgtaggcgcgacttgctggccaaatctattgagttgtagatgtggatgaatttgcactaaaaaaaaaaaaaa

SEQ ID NO: 20 - protein, 171 aa, Oryza sativa
GISDIITIPGLVNVDFADVKAVMKNSGTAMLGVGVSSSKNRAQEAARQATLAPLIGSSIEAATGVVYNITGGKDITLQEVNKVSQIVTSLADPSANIIFGAVVDDRYTGEIHVTIIATGFPQSFQKSLLADPKGARIMEAKEKAANLTYKAVAAATVQPAPAATWSRRLFS

SEQ ID NO: 21 - DNA, 2271 bp, Zea mays
ctaatagttttttctcatgcaaactatttatttctaaggtatgtgatgagtcctccaatctgagaagcagggcatggtaaaacaactcggcaggaatcaagagaaaccatagccactgccctgagggattcagatcttatcttcataacagctgggatgggagggggttctagatctggtgctgctccagttgttccccagatatcaaaggaagccggttatcttacagttggtgttgtcacctatccattcagtttcgagggccgtaagcgctctgtacaggcaagtatttgagcccccttcactcctgaattagaattcaaattgtcatatctcgttctgcgactttcttttgttcgatggaagcattagtttgtagtcataacaatgacatgcagccacatttattgcgatcatgtatataatggtagatcaaagaaatgtagcatcatgccatcacctgtagctcatctcataatttttgttcctacttttcttcgtggttgatgcccaaaacaatatacaactatgtggaatctattctaattaatccatgatttacctatgtgattgcaacagtaaacatatgataaccacatattaattaggtttaatagattcatttcacaaatcagtcactgtttatgcaattagttttataataaactcatgtttaatcattctaatcgaaatgcaaacatcggatgtgacccagactaaagttcagtccgagatccaaacaactcctacgcctgaagtcacagatcccacaaaactaggtgtagaaccaaaacaaagctgtacctaaatcgaatattttgatttaaaacgattttatttgtcaattagcgtgtttatctatctatggagttcagattcaaaagcgtgccatctctggaacctcccacccattctggagtctggacgcacgactaacggaattgcagaacgtgaagcttgccgctgagcgttgacctttgaggtacaaccatagaagatacaaatccccgactctttttgttctgttcatgtggatggcaataaaaactactgcaagttgcggatggacacggccagagaagcctcccatgttctagctagagttactaagcaggtcagctttatttcagcaggagtatagtaataaaaaaagagaggaagagagcggagattggtatggaagctttaccgcagctaccctggtccttgacctcggcgacagcacgtgtcattatgaatattcccccctgtggttaactgggaatacatttcactgttattacttttgaatttctatctgcaggtttttggccaatattccctttctaaacactttttgtttctgttctctatagttacttaatgttatgacttgtgtatgcccattttaagcattggaagcactagagaagctggaaaagagtgtagatacacttattgtgattccaaatgataagttattagatgttgccgatgaaaacatgcccttgcaagatgcatttctctttgcagatgatgttcttcgtcagggtgtccaaggaatatcagacatcatcacagtgggtggttctcccctttcctgctctaacttatctgcaaattgttatcatgtaccttatgagtgaacattgcagatatcaggacttgtcaatgttgattttgctgatgtaaaagctgtcatgaaaaactctggaacttccatgctcggtgttggtgtttcttccagcaaaatttgggcctaagaagctgctgaacaggcaacacttgctactttgattgggtcatccatcgaggcagctactggcgttgtgtataatattactggtgggaaggacatcactttgcaagaagtgaacaaggtgtcccaggtgcgtgtaggattccttagaaattctttattgattctgcaatggtgttttaagagaagttaggaaacgttgggtgtatcaaatagaagaaccaaatatatagttgttttagatagttttgaccatatgtattcacctcttgcagattgtgacaagcctagctggcccatctgcgaacataatttttggtgctgtcgttgatgaccgttacactggtgagatacatgtgacaatcactgcgacgggatttccacagtgcttccagaaatcccttttggcggatccaaagggagcatgtatagtggaatccaaagagaaaacaacaaccctcgcccataaagcagcagcagctacagttcaaccggtccctgcttctacttggtctcgaagactcttctcctga

SEQ ID NO: 22 - DNA, 30 bp, Artificial Sequence
  Description of Artificial Sequence PCR primer
acgtggatccaatgckgtkaatmgkatgat

SEQ ID NO: 23 - DNA, 27 bp, Artificial Sequence
  Description of Artificial Sequence PCR primer
acgtggatccgckccgaakatkakgtt

SEQ ID NO: 24 - DNA, 35 bp, Artificial Sequence
  Description of Artificial Sequence PCR primer
tagcggatccgtggcagtggcttgcagggtgttga

SEQ ID NO: 25 - DNA, 34 bp, Artificial Sequence
  Description of Artificial Sequence PCR primer
actgggatccakggatcagccaggctkgtgacaa

SEQ ID NO: 26 - DNA, 32 bp, Artificial Sequence
  Description of Artificial Sequence PCR primer
actgggatcctggatcmgcmaamswmgtmacm

SEQ ID NO: 27 - DNA, 31 bp, Artificial Sequence
  Description of Artificial Sequence PCR primer
gctaggatccggkttkcagggkgtkgatcck

SEQ ID NO: 28 - DNA, 37 bp, Artificial Sequence
  Description of Artificial Sequence PCR primer
agtcggatccatggccaccatgttaggactctcaaac

SEQ ID NO: 29 - DNA, 37 bp, Artificial Sequence
  Description of Artificial Sequence PCR primer
agtcggatccatggccaccatctcaaacccagcagag

SEQ ID NO: 30 - DNA, 37 bp, Artificial Sequence
  Description of Artificial Sequence PCR primer
acgtggatccctaaaagaacagcctccgagtaggtgt

SEQ ID NO: 31 - DNA, 36 bp, Artificial Sequence
  Description of Artificial Sequence PCR primer
ctggagatctatggctacttgtacatcagctgtgtt

SEQ ID NO: 32 - DNA, 36 bp, Artificial Sequence
  Description of Artificial Sequence PCR primer
ctagagatctatgcctcctgatacgcgacggtcacg

SEQ ID NO: 33 - DNA, 37 bp, Artificial Sequence
  Description of Artificial Sequence PCR primer
agtcagatcttcttaagctgttgggtagcgtgatcgc

SEQ ID NO: 34 - DNA, 26 bp, Artificial Sequence
  Description of Artificial Sequence PCR primer
catcactaatgacagttgcggtgcaa

SEQ ID NO: 35 - DNA, 27 bp, Artificial Sequence
  Description of Artificial Sequence PCR primer
ataatcatcgcaagaccggcaacagga

SEQ ID NO: 36 - DNA, 20 bp, Artificial Sequence
  Description of Artificial Sequence PCR primer
ggtgctcctgtaattgctgg

SEQ ID NO: 37 - DNA, 21 bp, Artificial Sequence
  Description of Artificial Sequence PCR primer
catttcctccagtgatattcc

SEQ ID NO: 38 - protein, 14 aa, Artificial Sequence
  Description of Artificial Sequence synthetic polypeptide
EGRKRSLQALEAIE

SEQ ID NO: 39 - protein, 14 aa, Artificial Sequence
  Description of Artificial Sequence synthetic polypeptide
RRRAVQAQEGIAAL

SEQ ID NO: 40 - DNA, 23 bp, Artificial Sequence
  Description of Artificial Sequence PCR primer
tcctcttttaggggaacaggcag

SEQ ID NO: 41 - DNA, 24 bp, Artificial Sequence
  Description of Artificial Sequence PCR primer
cttcagctcggttcttgcttgatg

SEQ ID NO: 42 - DNA, 24 bp, Artificial Sequence
  Description of Artificial Sequence PCR primer
tgacaaattattgacagctgtttc

SEQ ID NO: 43 - DNA, 24 bp, Artificial Sequence
  Description of Artificial Sequence PCR primer
acattaactagcccaggaatcgta

SEQ ID NO: 44 - DNA, 24 bp, Artificial Sequence
  Description of Artificial Sequence PCR Primer
tgatccctctgctaacatcatatt

SEQ ID NO: 45 - DNA, 24 bp, Artificial Sequence
  Description of Artificial Sequence PCR Primer
acagcctccgagtaggtgtccgtg

SEQ ID NO: 46 - DNA, 24 bp, Artificial Sequence
  Description of Artificial Sequence PCR Primer
ttgtacatcagctgtgtttatgcc

SEQ ID NO: 47 - DNA, 20 bp, Artificial Sequence
  Description of Artificial Sequence PCR Primer
atccaccacctcctacacca

(19) United States
(12) Patent Application Publication
(10) Pub. No.: US 2003/0177532 A1
(43) Pub. Date: Sep. 18, 2003

(54) Manipulation of starch granule size and number

(76) Inventors: Michael Meyrick Burrell, Cambridge (GB); Stephen Andrew Coates, Cambridge (GB)

Correspondence Address: PENNIE AND EDMONDS, 1155 AVENUE OF THE AMERICAS, NEW YORK, NY, 100362711

(21) Appl. No.: 10/279,579

(22) Filed: Oct. 24, 2002

Related U.S. Application Data
(60) Provisional application No. 60/346,905, filed on Jan. 8, 2002.

(30) Foreign Application Priority Data
Oct. 24, 2001 (GB) 0125493.7

(51) Int. Cl. A01H 1/00; C12N 5/04; C12N 15/82; C12Q 1/68; C07H 21/04; C12N 9/24

(52) U.S. Cl. 800/284; 435/6; 435/69.1; 435/200; 435/419; 536/23.2

(57) ABSTRACT

The invention provides isolated nucleic acids which encompass FtsZ nucleic acid molecules, FtsZ protein products (including, but not limited to, transcriptional products such as mRNAs, antisense and ribozyme molecules, and translational products such as FtsZ proteins, polypeptides, peptides and fusion proteins related thereto), antibodies to FtsZ protein products, vectors and expression vectors with FtsZ nucleic acids, cells, plants and plant parts with FtsZ nucleic acids, modified starch and starch granules from such plants and the use of the foregoing to improve agronomically valuable plants, including but not limited to maize, wheat, barley and potato.

This application claims priority to United States Provisional Patent Application No. 60/346,905, filed on Jan. 8, 2002 and Great Britain Patent Application No. 0125493.7, filed on Oct. 24, 2001, both of which are incorporated by reference herein in their entireties.

### 1. FIELD OF INVENTION

The present invention is based upon the identification of a protein, which alters the sizes and quantity of starch granules in a plant. In particular, the invention relates to FtsZ nucleic acid molecules, FtsZ gene products, antibodies to FtsZ gene products, vectors and expression vectors with FtsZ genes, cells, plants and plant parts with FtsZ genes, modified starch, and starch granules from such plants and the use of the foregoing to improve agronomically valuable plants.

### 2. BACKGROUND

Starch, a branched polymer of glucose consisting of largely linear amylose and highly branched amylopectin, is the product of carbon fixation during photosynthesis in plants, and is the primary metabolic energy reserve stored in seeds and fruit. For example, up to 75% of the dry weight of grain in cereals is made up of starch. The importance of starch as a food source is reflected by the fact that two thirds of the worlds food consumption (in terms of calories) is provided by the starch in grain crops such as wheat, rice and maize.

Starch is the product of photosynthesis, and is analogous to the storage compound glycogen in eukaryotes. It is produced in the chloroplasts or amyloplasts of plant cells, these being the plastids of photosynthetic cells and non-photosynthetic cells, respectively. The biochemical pathway leading to the production of starch in leaves has been well characterised, and considerable progress has also been made in elucidating the pathway of starch biosynthesis in storage tissues.

The biosynthesis of starch molecules is dependent on a complex interaction of numerous enzymes, including several essential enzymes such as ADP-Glucose, a series of starch synthases which use ADP glucose as a substrate for forming chains of glucose linked by alpha-1-4 linkages, and a series of starch branching enzymes that link sections of polymers with alpha-1-6 linkages to generate branched structures (Smith et al., 1995, Plant Physiology, 107:673-677). Further modification of the starch by yet other enzymes, i.e. debranching enzymes or disproportionating enzymes, can be specific to certain species.

The fine structure of starch is a complex mixture of D-glucose polymers that consist essentially of linear chains (amylose) and branched chains (amylopectin) glucans. Typically, amylose makes up between 10 and 25% of plant starch, but varies significantly among species. Amylose is composed of linear D-glucose chains typically 250-670 glucose units in length (Tester, 1997, in: Starch Structure and Functionality, Frazier et al., eds., Royal Society of Chemistry, Cambridge, UK). The linear regions of amylopectin are composed of low molecular weight and high molecular weight chains, with the low ranging from 5 to 30 glucose units and the high molecular weight chains from 30 to 100 or more. The amylose/amylopectin ratio and the distribution of low and high molecular weight D-glucose chains can affect starch granule characteristics such as gelatinization temperature, retrogradation, and viscosity (Blanshard, 1987.) The characteristics of the fine structure of starch mentioned above have been examined in detial and are well known in the art of starch chemistry.

Starch granules extracted from rice are typically polygonal in shape and ranging from 3 to 8 um in diameter, maize has both polygonal and round granules ranging from 5 to 25 um in diameter with an average of 15 um, and tapioca (Manihot or cassava) starch granules typically have rounded shapes truncated at one end averaging 20 um in diameter, but ranging from 5 to 35 um. The starch of wheat and other cereal crops has predominantly round starch granules, with some flat granules and elliptical granules that are categorized into two types, large and small granules. The starch of potato comprises the largest commercially available granules which are oval or egg shaped and range from 15-100 um in diameter (Wurzburg, 1986, Modified starches: properties and uses, CRC Press, Boca Raton, Fla.).

Starch molecules are deposited in successive layers around a central hilum and through hydrogen bonding to form a tightly packed granule. The starch molecules are arranged radially to form a partially crystalline structure that causes polarized light passed through the granule to exhibit bifringence. The outer amorphous areas have weaker and/or fewer hydrogen bonds holding the starch molecules together. The inner, micellar or crystalline layers, areas have stronger bonds.

The fine structure of starch can be correlated to some extent to the structure of starch granules. It is know that starch granule size and amylose percentage change during kernel development in maize and during tobacco leaf development (Boyer et al., 1976, Cereal Chem 53:327-337). In his classic study Boyer et al. concluded the amylose percentage of starch decreases with decreasing granule size in later stages of maize kernel development. Another way in which the fine structure of starch can be correlated to the structure of starch granules is through the organization of amylose and amylopectin in granules. The two molecules form alternating semi-crystalline and amorphous layers, the layers in most starches having central symmetry. The semi-crystalline layers consist of ordered regions composed of double helices formed by short amylopectin branches, most of which are further ordered into crystalline structures. The amorphous regions of the semi-crystalline layers and the amorphous layers are composed of amylose and non-ordered amylopectin branches. There is an additional complexity relating to the nature of the crystalline structures. The double helices comprising the crystallites may be densely packed in an orthgonal pattern, as in cereal starches, or less densely packed in an hexagonal pattern, as in potato starch. Both types of crystallite contain structural water, the amount and mobility of which is greater in potato-type crystallites. Starches from other species, for example pea, contain both types of crystallites, the two types of crystallite being confined to specific regions of the granule.

The production of starch comprising granules of a more uniform size would reduce the need for, and cost of, post-harvest processing. Such starch would have more uniform gelling properties. In wheat the elimination of the smaller granules would improve starch extractability. Furthermore, it has recently been discovered that the proportion of smaller granules influences water absorption and hence the water content of dough, an important quality in bread making. Additionally, the size and relative number of starch granules can effect several characteristics of starch including gelatinization temperature, retrogradation, and viscosity. Starch modified with respect to these characteristics can be used in commercial food products, industrial products, paper products, textile warp additives, and corrugating and adhesive industries. Specific products made from such modified starch include, but are not limited to, viscoelastic starch pastes, starch gels, thermoplasts, and extruded starch foams.

Although the biochemical pathway leading to the production of starch in leaves and storage organs has been extensively studied, the processes involved in the initiation and control of granule size are not understood. There is therefore an interest in, and a need for, a method of modifying the number and/or size of starch granules in plants which has not been met by the prior art.

Starch is synthesized in amyloplasts, which are committed primarily to starch production in storage organs such as the potato tuber and cereal endosperm are called amyloplasts. Among the various different types of plastids present in plants, chloroplasts have been studied most extensively because of their role in photosynthesis. The morphology and population dynamics of chloroplast division have been well documented, but comparatively little is known about the molecular controls underlying chloroplast division. It is thought that chloroplasts were originally prokaryotic endosymbionts, and division of chloroplasts is superficially similar to that of bacteria. For this reason it has been proposed that knowledge of plant homologues of bacterial cell division genes may be essential for understanding the process of chloroplast division in full (Pyke, 1997, American Journal of Botany 84: 1017-1027)

Several genes essential for cell division in prokaryotes have been identified. One of these encodes the protein FtsZ, which forms a ring at the leading edge of the cell division site. Two genes have been identified in Arabidopsis which encode proteins with significant sequence homology to E. coli FtsZ (Osteryoung and Vierling (1995) Nature, 376, 473-474; Osteryoung et al. (1998) The Plant Cell 10: 1991-2004). AtFtsZ1-1 contains a chloroplast targeting sequence while AtFtsZ2-1 was thought to be localized in the cytosol. A second gene closely related to AtFtsZ2-1 has also been identified in Arabidopsis, designated AtFtsZ2-2, leading to the hypothesis that there are two functionally divergent FtsZ gene families in plants, encoding differentially localized gene products (Osteryoung et al. (1998)). In subsequently published work (McAndrew et al. (2001)), it has been demonstrated that the original sequences designated as AtFtsZ2-1 and AtFtsZ2-2 were not full length and that in fact both of the products of these genes do have chloroplast targeting transit peptide sequences allowing for the import of the proteins into the chloroplast and a functional interaction with the product of the AtFtsZ1-1 protein.

Antisense down regulation of either Arabidopsis FtsZ gene (AtFtsZ1 -1 or AtFtsZ2-1) in transgenic Arabidopsis showed that both genes are essential for chloroplast division (WO 98/00436; Osteryoung et al. (1998)). It was further showed that a single FtsZ sequence, FtsZ1 could alter plastid division (Osteryoung et al. U.S. Pat. No.: 5,981,836 (1999)). In contrast, overexpression of the two genes gave different results. Transgenic plants overexpressing AtFtsZ1-1 showed inhibited chloroplast division and in some cases novel chloroplast morphology while those overexpressing AtFtsZ2-1 did not show any obvious effect on chloroplast division or morphology (Stokes et al. (2000) Plant Physiol. 124: 1668-1677).

However, there is no indication or suggestion in the prior art that FtsZ genes, can be used to alter the number and/or size of starch granules in plants.

### 3 SUMMARY OF THE INVENTION

The invention provides isolated nucleic acids which encompass FtsZ nucleic acid molecules, FtsZ protein products (including, but not limited to, transcriptional products such as mRNAs, antisense and ribozyme molecules, and translational products such as FtsZ proteins, polypeptides, peptides and fusion proteins related thereto), antibodies to FtsZ protein products, vectors and expression vectors with FtsZ nucleic acids, cells, plants and plant parts with FtsZ nucleic acids, modified starch from such plants and the use of the foregoing to improve agronomically valuable plants, including but not limited to maize, wheat, barley and potato.

The invention is based upon the identification of a protein responsible for controlling starch granule size. In particular, the inventors have discovered nucleic acid molecules from wheat and potato which have sequences that are homologous to the known FtsZ genes of Arabidopsis. FtsZ genes from other plant species have been identified by analysis of sequence homology with the wheat and potato sequences of the invention.

Altering the numbers, sizes, and distributions of starch granules allows various characteristics and properties of starch to be regulated. By altering aspects of starch related to starch granules, the starch extracted from the plant may be altered in magnitude and directions that may be more favorable for nutritional or industrial uses.

The present invention provides for an isolated nucleic acid molecule that comprises a nucleotide sequence which encodes a polypeptide comprising the amino acid sequence that is at least 86% to 98% identical to SEQ ID NO: 2, 4, 6, 8, 10, 12, 14, 16, 18, or 20, or a fragment thereof as determined using the BLASTX or program with a score=50 and wordlength=3; comprises a nucleotide sequence at least 83% to 94% identical to SEQ ID NOs: 1, 3, 5, 7, 9, 11, 13, 15, 17, 19, or 21 or a complement thereof as determined using the BLASTN program with a score=100 and wordlength=12; or hybridizes to a nucleic acid molecule consisting of SEQ ID NOs: 1, 3, 5, 7, 9, 11, 13, 15, 17, 19, or 21 or a complement thereof, under conditions of hybridization comprising washing at 60° C. twice for 15 minutes in 2×SSC, 0.5% SDS.

In another embodiment the percent identity of two nucleotide sequences can be determined using the BESTFIT or GAP programs with a gap weight of 50 and a length weight of 3, and the percent identity of two polypeptide sequences using the BESTFIT or GAP programs with a gap weight of 12 and a length weight of 4.

In one embodiment, the invention provides for a fragment of any one of the isolated nucleic acid molecules encompassed by the invention as described herein wherein the fragment comprises at least 40, 60, 80, 100 or 150 contiguous nucleotides of the nucleic acid molecule of the invention.

The invention provides for an isolated polypeptide comprising, an amino acid sequence that is at least 86-98% identical to SEQ ID NO: 2, 4, 6, 8, 10, 12, 14, 16, 18, or 20 or a fragment thereof, an amino acid sequence encoded by any one of the nucleic acid molecules encompassed by the invention as described herein; or an amino acid sequence of SEQ ID NO: 2, 4, 6, 8, 10, 12, 14, 16, 18, or 20 or a fragment thereof.

The invention provides for a polypeptide comprising the amino acid sequence of SEQ ID NO: 2, 4, 6, 8, 10, 12, 14, 16, 18, or 20 and which further comprises one or more conservative amino acid substitution.

The invention provides for a fusion polypeptide comprising any one of the amino acid sequences encompassed by the invention as described herein and a heterologous polypeptide.

The invention provides for a fragment or immunogenic fragment of any one of the polypeptides encompassed by the invention as described herein, wherein the fragment comprises at least 8, 10, 15, 20, 25, 30 or 35 consecutive amino acids of the polypeptide.

The invention provides for a method for making any one of the polypeptides encompassed by the invention as described herein, comprising the steps of culturing a cell comprising a recombinant polynucleotide encoding the polypeptide of any one of the polypeptides encompassed by the invention as described herein, under conditions that allow said polypeptide to be expressed by said cell; and recovering the expressed polypeptide.

The invention provides for a vector comprising of any one of the nucleic acid molecules encompassed by the invention as described herein.

The invention provides for an expression vector comprising of any one of the nucleic acid molecules encompassed by the invention as described herein, including sense and/or antisense molecules, and at least one regulatory region operably linked to the nucleic acid molecule. The invention provides for the expression vector as described above, wherein the regulatory region confers chemically-inducible, dark-inducible, developmentally regulated, developmental-stage specific, wound-induced, environmental factor-regulated, organ-specific, cell-specific, and/or tissue-specific expression of the nucleic acid molecule, or constitutive expression of the nucleic acid molecule. The invention provides for the expression vector as described above, wherein the regulatory region is selected from the group consisting of a 35S CaMV promoter, a rice actin promoter, a patatin promoter, and a high molecular weight glutenin gene of wheat.

The invention provides for an expression vector comprising the antisense molecules of any one of the nucleic acid molecules encompassed by the invention as described herein, wherein the antisense sequence is operably linked to at least one regulatory region.

The invention provides for a genetically-engineered cell which comprises of any one of the nucleic acid molecules encompassed by the invention as described herein. In a related embodiment, a cell comprises any one of the above described expression vectors.

The invention provides for a genetically-engineered plant or progeny thereof comprises any one of the above described expression vectors and further comprising of any one of the nucleic acid molecules encompassed by the invention as described herein. In a related embodiment, in a genetically-engineered plant as described above the nucleic acid molecule comprises an antisense nucleotide sequence.

The invention provides for a plant part from any one of the genetically-engineered plants described above comprising of any one of the nucleic acid molecules encompassed by the invention as described herein, wherein the overall size of starch granules is altered relative to a plant part not comprising the nucleic acid molecule. In one embodiment, the plant part described above is a tuber, stem, root, seed, or seed endosperm.

The invention also provides for starch granules obtained from any one of the genetically-engineered plants described above, wherein at least one of the starch granules is larger than any of the granules found in a plant without the nucleic acid molecule. Starch granules obtained from any one of the genetically-engineered plants described above, wherein the starch granules are larger than any found in the plant without the nucleic acid molecule.

In one embodiment, the invention provides for a method of altering the sizes of starch granules comprising introducing into a plant any one of the expression vectors encompassed by the invention described herein, and growing the plant such that the nucleic acid molecule in the expression vector is expressed, wherein the size of the starch granules is altered relative to a plant without the expression vector. In a related embodiment, the invention provides for a method of altering the sizes of starch granules comprising introducing into a plant any one of the expression vectors encompassed by the invention described herein, and growing the plant such that the nucleic acid molecule in the expression vector is expressed, wherein the size of one or more starch granule is larger than any found in the plant without the expression vector. In another related embodiment, the invention provides for a method of altering the sizes of starch granules comprising introducing into a plant any one of the expression vectors encompassed by the invention described herein, and growing the plant such that the nucleic acid molecule in the expression vector is expressed, wherein altering the sizes of starch granules results in an increase in a ratio of large to small starch granules.

In another embodiment, the invention provides for a method of altering the sizes of starch granules comprising introducing into a plant any one of the expression vectors encompassed by the invention described herein, and growing the plant such that the nucleic acid molecule in the expression vector is expressed, wherein the size of the starch granules is altered relative to a plant without the expression vector. In a related embodiment, the invention provides for a method of altering the sizes of starch granules comprising introducing into a plant any one of the expression vectors encompassed by the invention described herein, and growing the plant such that the nucleic acid molecule in the expression vector is expressed, wherein altering the sizes of starch granules results in an decrease in a ratio of large to small starch granules. In another related embodiment, the invention provides for a method of altering the sizes of starch granules comprising introducing into a plant any one of the expression vectors encompassed by the invention described herein, and growing the plant such that the nucleic acid molecule in the expression vector is expressed, wherein the small starch granules are less than or equal to 10 um in diameter and the large starch granules are greater than 10 um in diameter. In yet another related embodiment, the invention provides for a method of altering the sizes of starch granules comprising introducing into a plant any one of the expression vectors encompassed by the invention described herein, and growing the plant such that the nucleic acid molecule in the expression vector is expressed, wherein altering the sizes of starch granules results in a shift in a distribution of starch granule size towards larger or smaller granules.

In another embodiment, the invention provides for a method of altering the sizes of starch granules comprising introducing into a plant any one of the expression vectors encompassed by the invention described herein, and growing the plant such that the nucleic acid molecule in the expression vector is expressed, wherein altering the sizes of starch granules results in a shift in a distribution of starch granule size, wherein a peak in the distribution widens. The invention also provides for a method of making starch granules comprising, growing a plant comprising any one of the nucleic acids encompassed by the invention described herein, such that the overall size of the starch granules is altered relative to that of a plant without the nucleic acid; and extracting the starch granules from the plant.

The invention provides for a method of altering one or more starch characteristics comprising growing a plant comprising any one of the nucleic acids encompassed by the invention described herein, such that the overall size of the starch granules is altered relative to that of a plant without the nucleic acid, wherein the characteristics of the starch from the plant with the nucleic acid is modified relative to a plant without the nucleic acid. The invention also provides for methods wherein the characteristic altered is selected from the group consisting of viscosity, gelling, thickness, foam density, or pasting.

The invention provides for a method for altering starch granule quantity comprising, introducing into a plant an expression vector of the present invention described herein, such that the quantity of starch granules is altered relative to a plant without the expression vector.

The invention also provides for the methods described herein for altering the sizes of starch granules, with the additional limitation that the viscosity of starch is increased or decreased.

In a preferred embodiment, the invention provides for a genetically-engineered potato cell comprising a patatin promoter operably linked to a nucleic acid molecule of SEQ ID NO: 1 such that said patatin promoter regulates transcription of said molecule, and wherein sizes of starch granules in the cell are altered relative to a potato cell not comprising the nucleic acid molecule.

In a preferred embodiment, the invention provides for a genetically-engineered potato cell comprising a patatin promoter operably linked to a nucleic acid molecule of SEQ ID NO: 9 in an antisense orientation, such that said patatin promoter regulates transcription of said molecule, and wherein sizes of starch granules in the cell are altered relative to a potato cell not comprising the nucleic acid molecule.

In another preferred embodiment, the invention provides for a genetically-engineered cereal cell comprising a HMWG promoter operably linked to a nucleic acid molecule of SEQ ID NO: 5 in an antisense orientation, such that said HMWG promoter regulates transcription of said molecule, and wherein sizes of starch granules in the cell exhibit an increase in a ratio of large to small granules relative to a cereal cell not comprising the nucleic acid molecule.

In yet another preferred embodiment, the invention provides for a plant derived from any one of the genetically-engineered cells described above and altered starch extracted from such plants and/or cells.

The invention also provides for altered starch extracted from genetically-engineered cells or plants as described herein comprising starch granules of a more uniform size and/or a population of starch granules from the plant of claim, wherein the size distribution is more uniform relative to a non-engineered control plant. In a preferred embodiment, the genetically-engineered cells or plants are of a cereal grain species and exhibit an alteration, i.e. increase or decrease in the ration of large (A type) to small (B type) starch granules.

3.1 Sequence Identifiers

The present invention is illustrated by way of non-limiting examples of biological sequences in which:

SEQ ID NO: 1 shows the nucleotide and predicted amino acid sequence for the first potato FtsZ2 fragment isolated by PCR.

SEQ ID NO: 2 shows the predicted amino acid sequence for the first potato FtsZ2 fragment isolated by PCR.

SEQ ID NO: 3 shows the nucleotide and predicted amino acid sequence for the second potato FtsZ2 fragment isolated by PCR.

SEQ ID NO: 4 shows the predicted amino acid sequence for the second potato FtsZ2 fragment isolated by PCR.

SEQ ID NO: 5 shows the nucleotide and predicted amino acid sequence for the first wheat FtsZ2 fragment isolated by PCR.

SEQ ID NO: 6 shows the predicted amino acid sequence for the first wheat FtsZ2 fragment isolated by PCR.

SEQ ID NO: 7 shows the nucleotide and predicted amino acid sequence for the second wheat FtsZ2 fragment isolated by PCR.

SEQ ID NO: 8 shows the predicted amino acid sequence for the second wheat FtsZ2 fragment isolated by PCR.

SEQ ID NO: 9 shows the nucleotide and predicted amino acid sequence for the potato FtsZ1 fragment isolated by PCR.

SEQ ID NO: 10 shows the predicted amino acid sequence for the potato FtsZ1 fragment isolated by PCR.

SEQ ID NO: 11 shows the nucleotide and predicted amino acid sequence for the full length potato FtsZ1 cDNA isolated by PCR.

SEQ ID NO: 12 shows the predicted amino acid sequence for the full length potato FtsZ1 cDNA isolated by PCR.

SEQ ID NO: 13 shows the nucleotide and predicted amino acid sequence for the full length potato FtsZ2 cDNA isolated by PCR.

SEQ ID NO: 14 shows the predicted amino acid sequence for the full length potato FtsZ2 cDNA isolated by PCR.

SEQ ID NO: 15 shows the nucleotide and predicted amino acid sequence for the wheat EST Accession No. SCU007.B07.R990714 which is identified as a fragment of wheat FtsZ.

SEQ ID NO: 16 shows the predicted amino acid sequence for the wheat EST Accession No. SCU007.B07.R990714.

SEQ ID NO: 17 shows the nucleotide and predicted amino acid sequence for the maize EST Accession No. AI745801 which is identified as a fragment of maize FtsZ.

SEQ ID NO: 18 shows the predicted amino acid sequence for the maize EST Accession No. AI745801.

SEQ ID NO: 19 shows the nucleotide and predicted amino acid sequence for the combined Rice EST's Accession No's. C27863 and AU091451 having homology to FtsZ1.

SEQ ID NO: 20 shows the predicted amino acid sequence for the combined Rice EST's Accession No's. C27863 and AU091451.

SEQ ID NO: 21 shows the nucleotide sequence for the maize genomic fragment Accession No. AF105716 which is identified as a fragment of maize FtsZ.

SEQ ID NO: 22 shows the nucleotide sequence for a PCR primer used to isolate FtsZ type 2 cDNA fragments.

SEQ ID NO: 23 shows the nucleotide sequence for a PCR primer used to isolate FtsZ type 2 cDNA fragments.

SEQ ID NO: 24 shows the nucleotide sequence for a PCR primer used to isolate FtsZ type 1 cDNA fragments.

SEQ ID NO: 25 shows the nucleotide sequence for a PCR primer used to isolate FtsZ type 1 cDNA fragments.

SEQ ID NO: 26 shows the nucleotide sequence for a PCR primer used to isolate FtsZ type 1 cDNA fragments.

SEQ ID NO: 27 shows the nucleotide sequence for a PCR primer used to isolate FtsZ type 1 cDNA fragments.

SEQ ID NO: 28 shows the nucleotide sequence for a PCR primer used to isolate FtsZ type 1 full length cDNA sequences.

SEQ ID NO: 29 shows the nucleotide sequence for a PCR primer used to isolate FtsZ type 1 full length cDNA sequences.

SEQ ID NO: 30 shows the nucleotide sequence for a PCR primer used to isolate FtsZ type 1 full length cDNA sequences.

SEQ ID NO: 31 shows the nucleotide sequence for a PCR primer used to isolate FtsZ type 2 full length cDNA sequences.

SEQ ID NO: 32 shows the nucleotide sequence for a PCR primer used to isolate FtsZ type 2 full length cDNA sequences.

SEQ ID NO: 33 shows the nucleotide sequence for a PCR primer used to isolate FtsZ type 2 full length cDNA sequences.

SEQ ID NO: 34 shows the nucleotide sequence for a PCR primer used to screen transformed potato plants.

SEQ ID NO: 35 shows the nucleotide sequence for a PCR primer used to screen transformed potato plants.

SEQ ID NO: 36 shows the nucleotide sequence for a PCR primer used to screen transformed barley plants.

SEQ ID NO: 37 shows the nucleotide sequence for a PCR primer used to screen transformed barley plants.

SEQ ID NO: 38 shows the synthetic peptide sequence used to produce antisera to FtsZ type 1 proteins.

SEQ ID NO: 39 shows the synthetic peptide sequence used to produce antisera to FtsZ type 2 proteins.

SEQ ID NO: 40 shows the nucleotide sequence for a PCR primer used in RT-PCR analysis of FtsZ type 1 expression.

SEQ ID NO: 41 shows the nucleotide sequence for a PCR primer used in RT-PCR analysis of FtsZ type 1 expression.

SEQ ID NO: 42 shows the nucleotide sequence for a PCR primer used in RT-PCR analysis of FtsZ type 2 expression.

SEQ ID NO: 43 shows the nucleotide sequence for a PCR primer used in RT-PCR analysis of FtsZ type 2 expression.

SEQ ID NO: 44 shows the nucleotide sequence for a PCR primer used in RT-PCR analysis of endogenous FtsZ type 1 expression.

SEQ ID NO: 45 shows the nucleotide sequence for a PCR primer used in RT-PCR analysis of endogenous FtsZ type 1 expression.

SEQ ID NO: 46 shows the nucleotide sequence for a PCR primer used in RT-PCR analysis of endogenous FtsZ type 2 expression.

SEQ ID NO: 47 shows the nucleotide sequence for a PCR primer used in RT-PCR analysis of endogenous FtsZ type 2 expression.

### 4 BRIEF DESCRIPTION OF THE FIGURES

FIG. 1 shows a map of the plasmid pFW14000, comprising the patatin promoter

FIG. 2 shows a map of the plasmid pFW14555, comprising the potato FtsZ2a fragment in sense orientation under the control of the patatin promoter

FIG. 3 shows a map of the plasmid pFW14556, comprising the potato FtsZ2a fragment in antisense orientation under the control of the patatin promoter

FIG. 4 shows a map of the plasmid pFW14561, comprising the potato FtsZ1 fragment in sense orientation under the control of the patatin promoter

FIG. 5 shows a map of the plasmid pFW14562, comprising the potato FtsZ1 fragment in antisense orientation under the control of the patatin promoter

FIG. 6 shows a map of the plasmid pDV03553, comprising the wheat FtsZ2a fragment in sense orientation under the control of the HMWG promoter

FIG. 7 shows a map of the plasmid pDV03554, comprising the wheat FtsZ2a fragment in antisense orientation under the control of the HMWG promoter

FIG. 8 shows a map of the plasmid pCL46B, comprising the wheat FtsZ2a fragment in sense orientation under the control of the HMWG promoter

FIG. 9 shows a map of the plasmid pCL47B, comprising the wheat FtsZ2a fragment in sense orientation under the control of the HMWG promoter

FIG. 10 shows a map of the plasmid GEX-FI+, comprising the potato full length FtsZ1 cDNA.

FIG. 11 shows a map of the plasmid GEX-F2+, comprising the potato full length FtsZ2 cDNA.

FIG. 12 shows a graph of the starch granule size distributions of starch extracted from barley endosperm transformed with pCL47B compared with starch extracted from control (non-transformed ) barley endosperm.

FIG. 13 shows a graph of the percentage of A type starch granules present in starch extracted from barley endosperm transformed with pCL47B compared with starch extracted from control (non-transformed ) barley endosperm.

FIG. 14 shows a cumulative frequency plot of the starch granule size distributions of starch extracted from potato microtuber tissue transformed with pFW14555 compared with starch extracted from control (non-co-cultivated) potato microtuber tissue.

FIG. 15 shows a cumulative frequency plot of the starch granule size distributions of starch extracted from potato microtuber tissue transformed with pFW14561 compared with starch extracted from control (non-co-cultivated) potato microtuber tissue.

FIG. 16 shows a cumulative frequency plot of the starch granule size distributions of starch extracted from potato microtuber tissue transformed with pFW14562 compared with starch extracted from control (non-co-cultivated) potato microtuber tissue.

FIG. 17 shows a cumulative frequency plot of the starch granule size distributions of starch extracted from potato tuber tissue transformed with pFW14555, pFW14562, or pFW14561 compared with starch extracted from control (non-co-cultivated) potato tuber tissue.

FIG. 18 shows the results from analysis of potato tuber starch from greenhouse grown tubers analyzed by Differential Scanning Calorimetry (DSC).

FIG. 19 shows the results of an RT-PCR using RNA from control and pFW14555 transformed tubers. Lane 1 is lamda/Pst1; lane 2 is Pr pFW14555-2; lane 3 is Pr NCC; lane 4 is no template (−ve control); lane 5 is plasmid pFW14555 template (+ve control); lane 6 is lamda/Pst1; lane 7 is lamda/Pst1; lane 8 is Pr pFW14555-2; lane 9 is Pr NCC; lane 10 is no template (−ve control); lane 11 is plasmid pFW14555 template (+ve control); lane 12 is lamda/Pst1. Products in lanes 2-5 were amplified with primer pair RT555F1 and RT555R2. The products in lanes 8-11 were amplified with primer pair RT565F1 and RT565R1.

FIGS. 20 shows the results of an RT-PCR using RNA from control and pFW14561 or 14562 transformed tubers. (A) Amplification using primer pair RT561F3 and RT561R3. Lane 1 is lamda/Pst1; lane 2 is Pr pFW14561-4; lane 3 is Pr pFW14561-13; lane 4 is Pr pFW14561-16; lane 5 is Pr pFW14562-5; lane 6 is Pr pFW14562-23; lane 7 is Pr pFW14562-28; lane 8 is Pr pFW14562-34; lane 9 is Pr pFW14562-38; lane 10 is Pr pFW14562-56; lane 11 is Pr NCC; lane 12 is no template (−ve control); lane 13 is plasmid pFW14561 template (+ve control); and lane 14 is lamda/Pst1. (B) Amplification using primer pair RT563F1 and RT563R1. Lane 16 is Pr pFW14561-4; lane 17 is Pr pFW14561-13; lane 18 is Pr pFW14561-16; lane 19 is Pr pFW14562-5; lane 20 is Pr pFW14562-23; lane 21 is Pr pFW14562-28; lane 22 is Pr pFW14562-34; lane 23 is Pr pFW14562-38; lane 24 is Pr pFW14562-56; lane 25 is Pr NCC; lane 26 is no template (−ve control); lane 27 is plasmid pAdV563 (full length FtsZI template (+ve control); and lane 28 is lamda/Pst1.

### 5. DETAILED DESCRIPTION OF THE INVENTION

5.1 FtsZ Nucleic Acids

The FtsZ polynucleotides or nucleic acids of the invention comprise a nucleotide sequence that is derived from plant species whose starch granules it is desired to alter, including but not limited to potato, wheat, maize, rice or barley. Other FtsZ nucleic acids that are characterized by their nucleotide sequence similarity to the FtsZ genes disclosed herein and/or to known FtsZ genes are also encompassed. The polynucleotides or nucleic acid molecules (the two terms are used interchangeably herein) of the invention can be DNA, RNA and comprise the nucleotide sequences of an FtsZ gene, or fragments or variants thereof from plants or other organisms. The terms nucleic acids, nucleic acid molecules, and polynucleotides are used interchangeably, and are intended to include DNA molecules (e.g., cDNA, genomic DNA), RNA molecules (e.g., hnRNA, pre-mRNA, mRNA, double-stranded RNA), and DNA or RNA analogs generated using nucleotide analogs. The polynucleotide can be single-stranded or double-stranded. An isolated polynucleotide is one which is distinguished from other polynucleotides that are present in the natural source of the polynucleotide. Preferably, an “isolated” polynucleotide lacks flanking sequences (i.e., sequences located at the 5′ and 3′ ends of the nucleic acid), which naturally flank the nucleic acid sequence in the genomic DNA of the organism from which the nucleic acid is derived.

In one embodiment, the FtsZ nucleic acids of the invention include the potato FtsZ2 sequence shown in SEQ ID NO: 1; the potato FtsZ2 sequence shown in SEQ ID NO: 3; the wheat FtsZ2 sequence shown in SEQ ID NO: 5; the wheat FtsZ2 sequence shown in SEQ ID NO: 7; the potato FtsZ1 sequence shown in SEQ ID NO: 9, the potato FtsZ1 cDNA sequence shown in SEQ ID NO: 11 and the potato FtsZ2 cDNA sequence shown in SEQ ID NO: 13, or fragments thereof, or sequences substantially homologous thereto. Also included are nucleic acid molecules encodes the amino acid sequence of SEQ ID NO: 2, 4, 6, 8, 10, 12, or 14, or a fragment or variant thereof. The variants may be an allelic variants or fragments thereof. Allelic variants being multiple forms of a particular gene or protein encoded by a particular gene. In various embodiments of the invention, an isolated polynucleotide that comprises the nucleic acid molecule of SEQ ID NO: 1, 3, 5, 7, 9, 11, 13, 15, 17, 19, or 21, or a complement, variant or fragment thereof is provided. In other embodiments, the nucleic acids of the invention comprise fragments of an FtsZ1 or FtsZ2 gene and regulatory elements of the gene such as promoters, enhancers, and transcription factor binding sites, wherein the fragments of the gene can correspond to a conserved domain, an exon, or a transit peptide. Antisense FstZ nucleic acids corresponding to the foregoing nucleic acids are also encompassed in the invention.

In a preferred embodiment, the nucleic acid molecules of the invention are comprised of full length sequences in that they encode an entire FtsZ protein as it occurs in nature. Examples of such sequences include SEQ ID NOs: 11 and 13. The corresponding amino acid sequences of full length FtsZ are SEQ ID NOs: 12 and 14. Preferably, the nucleic acids of the invention are isolated.

In various embodiments, the invention encompasses plant FstZ nucleic acids, including those from monocotyledonous and dicotyledonous plants, with the proviso that the plant FstZ nucleic acids do not consist of nucleotide sequences known in the art which include: 1. Arabidopsis thaliana; Accession Numbers Q425445, AL353912, AB052757.1 and AF089738. 2. Nicotiana tabacum; AJ271750, AJ133453, AJ271749, AJ271748, AF212159.5, AJ311847.1 and AF205858. 3. Gentiana lutea; AF205859. 4. Pisum sativum; T06774. 5. Tagetes erecta; AF251346. 6. Lilium longiflorum; AB042101. 7. Physcomitrella patens; AJ001586 and AJ249139. Although these nucleotide sequences are known in the art, their uses in the methods of the invention are not known and are thus encompassed in the invention. For example, genes that can be used in the methods of the invention include AtFtsZ1-1, AtFtsZ2-1 and AtFtsZ2-2 from Arabidopsis thaliana; NtFtsZ1-1, NtFtsZ1-2 and NtFtsZ1-3 from Nicotiana tabacum (Genbank accession numbers AJ272748, AJ133453 and AJ271749).

The nucleic acid molecules of the invention and their variants can be identified by several approaches including but not limited to analysis of sequence similarity and hybridization assays.

In the context of the present invention the term “substantially homologous,” “substantially identical,” or “substantial similarity,” when used herein with respect to sequences of nucleic acid molecules, means that the sequence has either at least 83% sequence identity with the reference sequence, preferably 84% sequence identity, more preferably at least 85%, 86%, 87%, 88%, 89%, 90%, 91%, 92%, 93% and most preferably at least 94% sequence identity with said sequences, in some cases the sequence identity may be 98% or more preferably 99%, or above, or the term means that the nucleic acid molecule is capable of hybridizing to the complement of the nucleic acid molecule having the reference sequence under stringent conditions.

In embodiments, the invention encompasses a nucleic acid sequence at least 92% identical to SEQ ID NO: 1, at least 92% identical to SEQ ID NO: 3, at least 83% identical to SEQ ID NO: 5, at least 83% identical to SEQ ID NO: 7, at least 94% identical to SEQ ID NO: 9, at least 92% identical to SEQ ID NO: 11, or at least 92% identical to SEQ ID NO: 13, as determined using BLASTN. In a less preferred embodiment, the invention encompasses a nucleic acid sequence at least 92% identical to SEQ ID NO: 1, at least 92% identical to SEQ ID NO: 3, at least 83% identical to SEQ ID NO: 5, at least 83% identical to SEQ ID NO: 7, at least 94% identical to SEQ ID NO: 9, at least 92% identical to SEQ ID NO: 11, or at least 92% identical to SEQ ID NO:13, as determined using BLASTN, wherein the sequences are not the FtsZ cDNA Arabidopsis sequences of Osteryoung (U.S. Pat. No. 5,981,836). In another less preferred embodiment, the invention encompasses a nucleic acid sequence at least 83% identical to SEQ ID NO: 5 or 7, wherein the nucleic acid sequence is not SEQ ID NO: 15.

“% identity”, as known in the art, is a measure of the relationship between two polynucleotides or two polypeptides, as determined by comparing their sequences. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. In one embodiment of the invention, the sequences are identical in length to those of the invention.

For example, sequences can be aligned with the software clustalw under Unix which generates a file with a “.aln” extension, this file can then be imported into the Bioedit program (Hall, T. A. 1999. BioEdit: a user-friendly biological sequence alignment editor and analysis program for Windows 95/98/NT. Nucl. Acids. Symp. Ser. 41:95-98) which opens the .aln file. In the Bioedit window, one can choose individual sequences (two at a time) and alignment them. This method allows for comparison of the entire sequences.

Methods for comparing the identity of two or more sequences are well known in the art. Thus for instance, programs available in the Wisconsin Sequence Analysis Package, version 9.1 (Devereux J et al, Nucleic Acids Res. 12:387-395, 1984, available from Genetics Computer Group, Maidson, Wis., USA). The determination of percent identity between two sequences can be accomplished using a mathematical algorithm. For example, the programs BESTFIT and GAP, may be used to determine the % identity between two polynucleotides and the % identity between two polypeptide sequences. BESTFIT uses the “local homology” algorithm of Smith and Waterman (Advances in Applied Mathematics, 2:482-489, 1981) and finds the best single region of similarity between two sequences. BESTFIT is more suited to comparing two polynucleotide or two polypeptide sequences which are dissimilar in length, the program assuming that the shorter sequence represents a portion of the longer. In comparison, GAP aligns two sequences finding a “maximum similarity” according to the algorithm of Neddleman and Wunsch (J. Mol. Biol. 48:443-354, 1970). GAP is more suited to comparing sequences which are approximately the same length and an alignment is expected over the entire length. Preferably the parameters “Gap Weight” and “Length Weight” used in each program are 50 and 3 for polynucleotides and 12 and 4 for polypeptides, respectively. Preferably % identities and similarities are determined when the two sequences being compared are optimally aligned.

Other programs for determining identity and/or similarity between sequences are also known in the art, for instance the BLAST family of programs (Karlin & Altschul, 1990, Proc. Natl. Acad. Sci. USA 87:2264-2268, modified as in Karlin & Altschul, 1993, Proc. Natl. Acad. Sci. USA 90:5873-5877, available from the National Center for Biotechnology Information (NCB), Bethesda, Md., USA and accessible through the home page of the NCBI at www.ncbi.nlm.nih.gov). These programs exemplify a preferred, non-limiting example of a mathematical algorithm utilized for the comparison of two sequences. Such an algorithm is incorporated into the BLASTN and BLASTX programs of Altschul, et al., 1990, J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTX program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Nucleotide sequences that have been identified according to this method include the wheat EST designated RHO:S:12674 shown in SEQ ID NO: 15 which shows homology to the AtFtsZ2 sequences; and the maize EST accession no. AI745801 (SEQ ID NO: 18), the overlapping rice ESTs C27863 and AU091451 (SEQ ID NO: 19), and the maize genomic clone AF105716 (SEQ ID NO: 21) which all show homology to the AtFtsZ1 sequence. The uses of these sequences in the methods of the invention are encompassed.

Another non-limiting example of a program for determining identity and/or similarity between sequences known in the art is FASTA (Pearson W. R. and Lipman D. J., Proc. Nat. Acac. Sci., USA, 85:2444-2448, 1988, available as part of the Wisconsin Sequence Analysis Package). Preferably the BLOSUM62 amino acid substitution matrix (Henikoff S. and Henikoff J. G., Proc. Nat. Acad. Sci., USA, 89:10915-10919, 1992) is used in polypeptide sequence comparisons including where nucleotide sequences are first translated into amino acid sequences before comparison.

Yet another non-limiting example of a program known in the art for determining identity and/or similarity between amino acid sequences is SeqWeb Software (a web-based interface to the GCG Wisconsin Package: Gap program) which is utilized with the default algorithm and parameter settings of the program: blosum 62, gap weight 8, length weight 2.

The percent identity between two sequences can be determined using techniques similar to those described above, with or without allowing gaps. In calculating percent identity, typically exact matches are counted.

Preferably the program BESTFIT is used to determine the % identity of a query polynucleotide or a polypeptide sequence with respect to a polynucleotide or a polypeptide sequence of the present invention, the query and the reference sequence being optimally aligned and the parameters of the program set at the default value.

Alternatively, variants and fragments of the nucleic acid molecules of the invention can be identified by hybridization to SEQ ID NOs: 1, 3, 5, 7, 9, 11, 13, 15, 17, 19, or 21. In the context of the present invention “stringent conditions” are defined as those given in Martin et al (EMBO J 4:1625-1630 (1985)) and Davies et al (Methods in Molecular Biology Vol 28: Protocols for nucleic acid analysis by non-radioactive probes, Isaac, P. G. (ed) pp 9-15, Humana Press Inc., Totowa N.J, USA)). The conditions under which hybridization and/or washing can be carried out can range from 42° C. to 68° C. and the washing buffer can comprise from 0.1×SSC, 0.5% SDS to 6×SSC, 0.5% SDS. Typically, hybridization can be carried out overnight at 65° C. (high stringency conditions), 60° C. (medium stringency conditions), or 55° C. (low stringency conditions). The filters can be washed for 2×15 minutes with 0.1×SSC, 0.5% SDS at 65° C. (high stringency washing). The filters were washed for 2×15 minutes with 0.1×SSC, 0.5% SDS at 63 ° C. (medium stringency washing). For low stringency washing, the filters were washed at 60° C. for 2×15 minutes at 2×SSC, 0.5% SDS.

In instances wherein the nucleic acid molecules are oligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may act as plant FtsZ gene antisense molecules, useful, for example, in FtsZ gene regulation and/or as antisense primers in amplification reactions of FtsZ gene and/or nucleic acid molecules. Further, such nucleic acid molecules may be used as part of ribozyme and/or triple helix sequences, also useful for FtsZ gene regulation. Still further, such molecules may be used as components in probing methods whereby the presence of a FtsZ allele may be detected.

In one embodiment, a nucleic acid molecule of the invention may be used to identify other FtsZ genes by identifying homologs. This procedure may be performed using standard techniques known in the art, for example screening of a cDNA library by probing; amplification of candidate nucleic acid molecules; complementation analysis, and yeast two-hybrid system (Fields and Song Nature 340 245-246 (1989); Green and Hannah Plant Cell 10 1295-1306 (1998)).

The invention also includes nucleic acid molecules, preferably DNA molecules, that are amplified using the polymerase chain reaction and that encode a gene product functionally equivalent to a FtsZ product.

In another embodiment of the invention, nucleic acid molecules which hybridize under stringent conditions to the nucleic acid molecules comprising a FtsZ gene and its complement are used in altering starch synthesis in a plant. Such nucleic acid molecules may hybridize to any part of a FtsZ gene, including the regulatory elements. Preferred nucleic acid molecules are those which hybridize under stringent conditions to a nucleic acid molecule comprising the nucleotide sequence encoding the amino acid sequence of SEQ ID NO: 2, 4, 6, 8, 10, 12, 14, 16, 18, or 20 and/or a nucleotide sequence of any one of SEQ ID NOs: 1, 3, 5, 7, 9, 11, 13, 15, 17, 19, or 21 or their complement sequences. In another embodiment of the invention, nucleic acid molecules are those which hybridize under stringent conditions to the nucleic acid molecules of SEQ ID NOs: 1, 3, 5, 7, 9, 11, 13, 15, 17, 19, or 21 hybridize over the full length of the sequences of the nucleic acid molecules. Preferably the nucleic acid molecule which hybridizes under stringent conditions to a nucleic acid molecule comprising the sequence of an FtsZ nucleic acid molecule of the invention or its complement are complementary to the nucleic acid molecule to which they hybridize.

Fragments of a FtsZ nucleic acid molecule of the invention preferably comprise, for example, in various embodiments, less than about 5 kb, 4 kb, 3 kb, 2 kb, 1 kb, 0.5 kb or 0.1 kb of nucleotide sequences which naturally flank the polynucleotide in genomic DNA of the cell from which the nucleic acid is derived. In other embodiments, the isolated FstZ polynucleotide is about 10-20, 21-50, 51-100, 101-200, 201-400, 401-750, 751-1000, 1001-1500 bases in length. Fragments of a FtsZ nucleic acid molecule of the invention encompassed by the invention may include introns and exons of FstZ genes, elements involved in regulating expression of the gene or may encode functional domains of FtsZ proteins. Fragments of the nucleic acid molecules of the invention encompasses fragments of SEQ ID NOs: 1, 3, 5, 7, 9, 11, 13, 15, 17, 19, or 21 as well as fragments of the variants of those sequences identified as defined above by percent homology or hybridization assay. Fragments of an FtsZ gene are preferably at least 40 nucleotides long, more preferably at least 60 nucleotides, at least 80 nucleotides, or most preferably at least 100 or 150 nucleotides in length, and may include elements involved in regulating expression of the gene.

The nucleic acid molecules of the invention which comprise or consist of an EST sequence can be used as probes for cloning corresponding full length genes. For example, the wheat EST of SEQ ID NO: 16 can be utilized as a probe in identifying and cloning the full length wheat homolog of the Arabidopsis FtsZ1 and FtsZ2 genes. The EST nucleic acid molecules may be used as sequence probes by themselves or in combination with the sequences of the invention in connection with computer software to search databases, such as GenBank for homologous sequences. Alternatively, the EST nucleic acid molecules can be used as probes in hybridization reactions as described herein. The EST nucleic acid molecules of the invention can also be used as molecular markers to map chromosome regions.

An isolated nucleic acid molecule encoding a variant protein can be created by introducing one or more nucleotide substitutions, additions or deletions into the FtsZ nucleic acid molecule, such that one or more amino acid substitutions, additions or deletions are introduced into the encoded protein. Mutations can be introduced by standard techniques, such as, ethyl methane sulfonate, X-rays, gamma rays, T-DNA mutagenesis, or site-directed mutagenesis, PCR-mediated mutagenesis. Briefly, PCR primers are designed that delete the trinucleotide codon of the amino acid to be changed and replace it with the trinucleotide codon of the amino acid to be included. This primer is used in the PCR amplification of DNA encoding the protein of interest. This fragment is then isolated and inserted into the full length cDNA encoding the protein of interest and expressed recombinantly.

An isolated nucleic acid molecule encoding a variant protein can be created by any of the methods described in section 5.1. Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, WH Freeman and Co.: 1995).

Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The invention also encompasses (a) DNA vectors that contain any of the foregoing nucleic acids and/or coding sequences (i.e. fragments and variants) and/or their complements (i.e., antisense molecules); (b) DNA expression vectors that contain any of the foregoing nucleic acids and/or coding sequences operatively associated with a regulatory region that directs the expression of the nucleic acids and/or coding sequences; and (c) genetically engineered host cells that contain any of the foregoing nucleic acids and/or coding sequences operatively associated with a regulatory region that directs the expression of the gene and/or coding sequences in the host cell. As used herein, regulatory region include, but are not limited to, inducible and non-inducible genetic elements known to those skilled in the art that drive and regulate expression of a nucleic acid. The nucleic acid molecules of the invention may be under the control of a promoter, enhancer, operator, cis-acting sequences, or trans-acting factors, or other regulatory sequence. The nucleic acid molecules encoding regulatory regions of the invention may also be functional fragments of a promoter or enhancer. The nucleic acid molecules encoding a regulatory region is preferably one which will target expression to desired cells, tissues, or developmental stages.

Examples of highly suitable nucleic acid molecules encoding regulatory regions are endosperm specific promoters, such as that of the high molecular weight glutenin (HMWG) gene of wheat, prolamin, or ITR1, or other suitable promoters available to the skilled person such as gliadin, branching enzyme, ADPG pyrophosphorylase, patatin, starch synthase, granule bound starch synthase, rice actin for example. Constitutive promoters may also be suitable. A suitable promoter in potato would be a tuber specific promoter, for example a promoter of the patatin gene family (Blundy K S; Blundy M A C; Carter D; Wilson F; Park W D; Burrell M M (1991), Plant Molecular Biology 16,153-160).

Other suitable promoters include the stem organ specific promoter gSPO-A, the seed specific promoters Napin, KTI 1, 2, & 3, beta-conglycinin, beta-phaseolin, heliathin, phytohemaglutinin, legumin, zein, lectin, leghemoglobin c3, ABI3, PvAlf, SH-EP, EP-C1, 2S 1, EM 1, and ROM2.

Constitutive promoters, such as CaMV promoters, including CaMV 35S and CaMV 19S may also be suitable. Other examples of constitutive promoters include Actin 1, Ubiquitin 1, and HMG2.

In addition, the regulatory region of the invention may be one which is environmental factor-regulated such as promoters that respond to heat, cold, mechanical stress, light, ultra-violet light, drought, salt and pathogen attack. The regulatory region of the invention may also be one which is a hormone-regulated promoter that induces gene expression in response to phytohormones at different stages of plant growth. Useful inducible promoters include, but are not limited to, the promoters of ribulose bisphosphate carboxylase (RUBISCO) genes, chlorophyll a/b binding protein (CAB) genes, heat shock genes, the defense responsive gene (e.g., phenylalanine ammonia lyase genes), wound induced genes (e.g., hydroxyproline rich cell wall protein genes), chemically-inducible genes (e.g., nitrate reductase genes, gluconase genes, chitinase genes, PR-1 genes etc.), dark-inducible genes (e.g., asparagine synthetase gene as described by U.S. Pat. No. 5,256,558), and developmental-stage specific genes (e.g., Shoot Meristemless gene, ABI3 promoter and the 2S1 and Em 1 promoters for seed development (Devic et al.,1996, Plant Journal 9(2):205-215), and the kin1 and cor6.6 promoters for seed development (Wang et al., 1995, Plant Molecular Biology, 28(4):619-634). Examples of other inducible promoters and developmental-stage specific promoters can be found in Datla et al., in particular in Table 1 of that publication (Datla et al., 1997, Biotechnology annual review 3:269-296).

A vector of the invention may also contain a sequence encoding a transit peptide which can be fused in-frame such that it is expressed as a fusion protein, such a sequence can be used to replace the native transit peptide of a FtsZ gene.

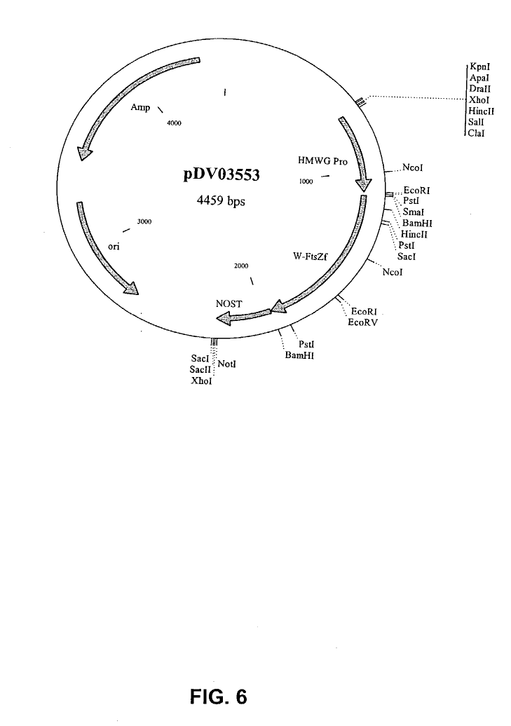

Methods which are well known to those skilled in the art can be used to construct vectors and/or expression vectors containing FtsZ protein coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, and Ausubel et al., 1989. Alternatively, RNA capable of encoding FtsZ protein sequences may be chemically synthesized using, for example, synthesizers. See, for example, the techniques described in Gait, 1984, Oligonucleotide Synthesis, IRL Press, Oxford. In a preferred embodiment of the invention, the techniques described in section 6, example 6, and illustrated in FIG. 6 are used to construct a vector.

A variety of host-expression vector systems may be utilized to express the FtsZ protein products of the invention. Such host-expression systems represent vehicles by which the FtsZ protein products of interest may be produced and subsequently recovered and/or purified from the culture or plant (using purification methods well known to those skilled in the art), but also represent cells which may, when transformed or transfected with the appropriate nucleic acid molecules, exhibit the FtsZ protein of the invention in situ. These include but are not limited to microorganisms such as bacteria (e.g., E. coli, B. subtilis) transformed with recombinant bacteriophage DNA, plasmid DNA or cosmid DNA expression vectors containing FtsZ protein coding sequences; yeast (e.g., Saccharomyces, Pichia) transformed with recombinant yeast expression vectors containing the FtsZ protein coding sequences; insect cell systems infected with recombinant virus expression vectors (e.g., baculovirus) containing the FtsZ protein coding sequences; plant cell systems infected with recombinant virus expression vectors (e.g., cauliflower mosaic virus, CaMV; tobacco mosaic virus, TMV); plant cell systems transformed with recombinant plasmid expression vectors (e.g., Ti plasmid) containing FtsZ protein coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter; the cytomegalovirus promoter/enhancer; etc.). In a preferred embodiment of the invention, an expression vector comprising a FtsZ nucleic acid molecule operably linked to at least one suitable regulatory sequence is incorporated into a plant by one of the methods described in this section, section 5.4, 5.5 and 5.6 or in examples 7, 8, 9, and 12.

In bacterial systems, a number of expression vectors may be advantageously selected depending upon the use intended for the FtsZ protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the FtsZ coding sequence may be ligated individually into the vector in frame with the lac Z coding region so that a fusion protein is produced; pIN vectors (Inouye & Inouye, 1985, Nucleic Acids Res. 13:3101-9; Van Heeke & Schuster, 1989, J. Biol. Chem. 264:5503-9); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

In one such embodiment of a bacterial system, full length cDNA nucleic acid molecules are appended with in-frame Bam HI sites at the amino terminus and Eco RI sites at the carboxyl terminus using standard PCR methodologies (Innis et al., 1990, supra) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson, et al., 1985, EMBO J. 4:1075; Zabeau and Stanley, 1982, EMBO J. 1: 1217).

The recombinant constructs of the present invention may include a selectable marker for propagation of the construct. For example, a construct to be propagated in bacteria preferably contains an antibiotic resistance gene, such as one that confers resistance to kanamycin, tetracycline, streptomycin, or chloramphenicol. Examples of other suitable marker genes include antibiotic resistance genes such as those conferring resistance to G4 18 and hygromycin (npt-II, hyg-B); herbicide resistance genes such as those conferring resistance to phosphinothricin and sulfonamide based herbicides (bar and suI respectively; EP-A-242246, EP-A-0369637) and screenable markers such as beta-glucoronidase (GB2 197653), luciferase and green fluorescent protein. Suitable vectors for propagating the construct include, but are not limited to, plasmids, cosmids, bacteriophages or viruses.

The marker gene is preferably controlled by a second promoter which allows expression in cells other than the seed, thus allowing selection of cells or tissue containing the marker at any stage of development of the plant. Preferred second promoters are the promoter of nopaline synthase gene of Agrobacterium and the promoter derived from the gene which encodes the 35S subunit of cauliflower mosaic virus (CaMV) coat protein. However, any other suitable second promoter may be used.

The nucleic acid molecule encoding a FtsZ protein may be native or foreign to the plant into which it is introduced. One of the effects of introducing a nucleic acid molecule encoding a FtsZ nucleic acid molecule into a plant is to increase the amount of FtsZ protein present and therefore the amount of starch produced by increasing the copy number of the nucleic acid molecule. Foreign FtsZ nucleic acid molecules may in addition have different temporal and/or spatial specificity for starch synthesis compared to the native FtsZ protein of the plant, and so may be useful in altering when and where or what type of starch is produced. Regulatory elements of the FtsZ nucleic acid molecules may also be used in altering starch synthesis in a plant, for example by replacing the native regulatory elements in the plant or providing additional control mechanisms. The regulatory regions of the invention may confer expression of a FtsZ nucleic acid molecules product in a chemically-inducible, dark-inducible, developmentally regulated, developmental-stage specific, wound-induced, environmental factor-regulated, organ-specific, cell-specific, tissue-specific, or constitutive manner. Alternatively, the expression conferred by a regulatory region may encompass more than one type of expression selected from the group consisting of chemically-inducible, dark-inducible, developmentally regulated, developmental-stage specific, wound-induced, environmental factor-regulated, organ-specific, cell-specific, tissue-specific, and constitutive.

Further, any of the nucleic acid molecules (including ESTs) and/or polypeptides and proteins described herein, can be used as markers for qualitative trait loci in breeding programs for crop plants. To this end, the nucleic acid molecules, including, but not limited to, full length FtsZ nucleic acid molecules coding sequences, and/or partial sequences (ESTs), can be used in hybridization and/or DNA amplification assays to identify the endogenous FtsZ nucleic acid molecules, FtsZ mutant alleles and/or FtsZ gene expression products in cultivars as compared to wild-type plants. They can also be used as markers for linkage analysis of qualitative trait loci. It is also possible that the FtsZ nucleic acid molecules may encode a product responsible for a qualitative trait that is desirable in a crop breeding program. Alternatively, the FtsZ protein and/or peptides can be used as diagnostic reagents in immunoassays to detect expression of the FtsZ nucleic acid molecules in cultivars and wild-type plants.

Genetically-engineered plants containing constructs comprising the FtsZ nucleic acid and a reporter gene can be generated using the methods described herein for each FtsZ nucleic acid gene variant, to screen for loss-of-function variants induced by mutations, including but not limited to, deletions, point mutations, rearrangements, translocation, etc. The constructs can encode for fusion proteins comprising a FtsZ protein fused to a protein product encoded by a reporter gene. Alternatively, the constructs can encode for a FtsZ protein and a reporter gene product that are not fused. The constructs may be transformed into a cell having the homozygous recessive FtsZ gene mutant background, and the restorative phenotype examined, i.e. quantity and quality of starch, as a complementation test to confirm the functionality of the variants isolated.

5.2 FtsZ Gene Products

In another aspect, the invention provides isolated FtsZ polypeptides, variants and fragments thereof (e.g., biologically active portions), as well as FtsZ peptides suitable for use as immunogens to raise antibodies directed against a FtsZ polypeptide of the invention.

In one embodiment, the native polypeptide can be isolated, using standard protein purification techniques, from cells or tissues expressing a FtsZ polypeptide. In a preferred embodiment, polypeptides of the invention are produced from expression vectors comprising FtsZ nucleic acid molecules as described in the previous section by recombinant DNA techniques. In another preferred embodiment, a polypeptide of the invention is synthesized chemically using standard peptide synthesis techniques.

The invention encompasses a polypeptide comprising an amino acid sequence of SEQ ID NOs: 2, 4, 6, 8, 10, 12, 14, 16, 18, or 20. Preferred polypeptides consist of an amino acid sequence of SEQ ID NOs: 2, 4, 6, 8, 10, 12, 14, 16, 18, or 20. The invention also encompasses FtsZ gene translational products which include, but are not limited to those proteins and polypeptides encoded by the sequences of the FtsZ nucleic acid molecules of the invention. The invention also encompasses proteins that are functionally equivalent to the FtsZ protein products of the invention. Such functionally equivalents of FtsZ proteins include polypeptides, peptides, fragments, variants, allelic variants, mutant forms of FtsZ proteins, truncated or deleted forms of FtsZ proteins, and FtsZ fusion proteins. The FtsZ proteins and functional equivalents can be prepared for a variety of uses, including, but not limited to, the manipulation of starch synthesis, generation of antibodies, use as reagents in assays, and identification of other cellular gene products involved in starch synthesis. The primary use of the FtsZ proteins and functional equivalents of the invention is to alter the number and size of starch granules found in storage portions of a plant.

An isolated or purified protein or biologically active portion thereof is substantially free of cellular material or other contaminating proteins from the cell or tissue source from which the protein is derived, or substantially free of chemical precursors or other chemicals when chemically synthesized. The language “substantially free” indicates protein preparations in which the protein is separated from cellular components of the cells from which it is isolated or recombinantly produced. Thus, protein that is substantially free of cellular material includes protein preparations having less than 20%, 10%, or 5% (by dry weight) of a contaminating protein.

Biologically active portions of a polypeptide of the invention include polypeptides comprising amino acid sequences identical to or derived from the amino acid sequence of the protein, such that the variants sequences comprise conservative substitutions or truncations (e.g., amino acid sequences comprising fewer amino acids than those shown in any of SEQ ID NO: 2, 4, 6, 8, 10, 12, 14, 16, 18, or 20, but which maintain a high degree of homology to the remaining amino acid sequence). Typically, biologically active portions comprise a domain or motif with at least one activity of the corresponding protein. Domains or motifs include, but are not limited to, a biologically active portion of a protein of the invention can be a polypeptide which is, for example, at least 10, 25, 50, 100, 200, 300, 400 or 500 amino acids in length.

In various embodiments, the invention also encompasses plant FstZ proteins and fragments thereof, including those from monocotyledonous and dicotyledonous plants, with the proviso that the plant FstZ proteins do not consist of amino acid sequences known in the art, including those that can be predicted from full length gene sequences such as those described in Section 5.1. Although these FtsZ proteins and fragments are known in the art, their uses in the methods of the invention are not known and are thus encompassed in the invention. In specific embodiments involving FtsZ polypeptides encoded by expressed sequence tags (ESTs), although the nucleotide sequences of the ESTs may be known, with no recognized function and reading frame information, such FtsZ polypeptides and their amino acid sequences are encompassed in the invention.

The present invention also provides variants of the polypeptides of the invention. Such variants may include but are not limited to homologs of the FtsZ proteins in other species, preferably plant species, and with the proviso that the species is not Arabidopsis thaliana. For example, other useful FtsZ proteins and polypeptides are substantially identical (e.g., at least 40%, preferably 50%, 60%, 65%, 75%, 85%, 90%, 95%, 96%, 97%, 98% or 99%) to any of SEQ ID NO: 2, 4, 6, 8, 10, 12, 14, 16, 18, or 20. In certain embodiments, the invention provides fragments of the amino acid sequence wherein the percent identity is determined over amino acid sequences of identical size to the fragment. In another embodiment, the invention encompasses an amino acid sequence at least 98% identical to SEQ ID NO: 2, at least 98% identical to SEQ ID NO: 4, at least 89% identical to SEQ ID NO: 6, at least 89% identical to SEQ ID NO: 8, at least 98% identical to SEQ ID NO: 10, at least 93% identical to SEQ ID NO: 12, or at least 88% identical to SEQ ID NO: 14, as determined using BLASTX. The percent identity can be determined over an amino acid sequence of identical size to said fragment. Determining whether two sequences are substantially similar may be carried out using any methodologies known to one skilled in the art, preferably using computer assisted analysis as described in section 5.1.

The FtsZ variants of the invention have an altered FtsZ amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, deleting one or more of the receiver domains. Thus, specific biological effects can be elicited by addition of a variant of limited function.

Modification of the structure of the subject polypeptides can be for such purposes as enhancing efficacy, stability, or post-translational modifications (e.g., to alter the phosphorylation pattern of the protein). Such modified peptides, when designed to retain at least one activity of the naturally-occurring form of the protein, or to produce specific antagonists thereof, are considered functional equivalents of the polypeptides. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule.

Whether a change in the amino acid sequence of a peptide results in a functional homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

The invention encompasses functionally equivalent mutant FtsZ proteins and polypeptides. The invention also encompasses mutant FtsZ proteins and polypeptides that are not functionally equivalent to the gene products. Such a mutant FtsZ protein or polypeptide may contain one or more deletions, additions or substitutions of FtsZ amino acid residues within the amino acid sequence encoded by any one the FtsZ nucleic acid molecules described above in Section 5. 1, and which result in loss of one or more functions of the FtsZ protein, thus producing a FtsZ gene product not functionally equivalent to the wild-type FtsZ protein.

FtsZ proteins and polypeptides bearing mutations can be made to FtsZ DNA (using techniques discussed above as well as those well known to one of skill in the art) and the resulting mutant FtsZ proteins tested for activity. Mutants can be isolated that display increased function, (e.g., resulting in improved root formation), or decreased function (e.g., resulting in suboptimal root function). Additionally, peptides corresponding to one or more exons of the FtsZ protein, truncated or deleted FtsZ protein are also within the scope of the invention. Fusion proteins in which the full length FtsZ protein or a FtsZ polypeptide or peptide fused to an unrelated protein are also within the scope of the invention and can be designed on the basis of the FtsZ nucleotide and FtsZ amino acid sequences disclosed herein.

While the FtsZ polypeptides and peptides can be chemically synthesized (e.g., see Creighton, 1983, Proteins: Structures and Molecular Principles, W. H. Freeman & Co., NY) large polypeptides derived from FtsZ gene and the full length FtsZ gene may advantageously be produced by recombinant DNA technology using techniques well known to those skilled in the art for expressing nucleic acid molecules.

Nucleotides encoding FtsZ proteins and fusion proteins may include, but are not limited to, nucleotides encoding full length FtsZ proteins, truncated FtsZ proteins, or peptide fragments of FtsZ proteins fused to an unrelated protein or peptide, such as for example, an enzyme, fluorescent protein, or luminescent protein that can be used as a marker or an epitope that facilitates affinity-based purification. A fusion protein of the invention can further comprise a heterologous polypeptide such as a transit peptide or fluorescence protein.

Further, it may be desirable to include additional DNA sequences in the protein expression constructs. Examples of additional DNA sequences include, but are not limited to, those encoding: a 3′ untranslated region; a transcription termination and polyadenylation signal; an intron; a signal peptide (which facilitates the secretion of the protein); or a transit peptide (which targets the protein to a particular cellular compartment such as the nucleus, chloroplast, mitochondria or vacuole). The nucleic acid molecules of the invention will preferably comprise a nucleic acid molecule encoding a transit peptide, to ensure delivery of any expressed protein to the plastid. Preferably, the transit peptide will be selective for amyloplasts, and can be native to the nucleic acid molecule of the invention or derived from known plastid sequences, such as those from the small subunit of the ribulose bisphosphate carboxylase enzyme (ssu of rubisco) from pea, maize or sunflower for example. Where an agonist or antagonist which modulates activity of the FtsZ protein is a polypeptide, the polypeptide itself must be appropriately targeted to the plastids, for example by the presence of plastid targeting signal at the N terminal end of the protein (Castro Silva Filho et al Plant Mol Biol 30 769-780 (1996) or by protein-protein interaction (Schenke P C et al, Plant Physiol 122 235-241 (2000) and Schenke et al PNAS 98(2) 765-770 (2001). The transit peptides of the invention are used to target transportation of FtsZ proteins as well as agonists or antagonists thereof to plastids, the sites of starch synthesis, thus altering the starch synthesis process and resulting starch characteristics.

The FtsZ proteins and transit peptides associated with the FtsZ genes of the present invention have a number of important agricultural uses. The transit peptides associated with the FtsZ genes of the invention may be used, for example, in transportation of desired heterologous gene products to a root, a root modified through evolution, tuber, stem, a stem modified through evolution, seed, and/or endosperm of transgenic plants transformed with such constructs.

The invention encompasses methods of screening for agents (i.e., proteins, small molecules, peptides) capable of altering the activity of a FtsZ protein in a plant. Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into nucleic acid molecules such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-33 1).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. In one embodiment, the antigenic peptide of a protein of the invention or fragments or immunogenic fragments of a protein of the invention comprise at least 8 (preferably 10, 15, 20, 30 or 35) consecutive amino acid residues of the amino acid sequence of SEQ ID NO: 2, 4, 6, 8, 10, 12, 14, 16, 18, or 20 and encompasses an epitope of the protein such that an antibody raised against the peptide forms a specific immune complex with the protein.

Exemplary amino acid sequences of the polypeptides of the invention can be used to generate antibodies against plant glycogenin-like genes. In one embodiment, the immunogenic polypeptide is conjugated to keyhole limpet hemocyanin (“KLH”) and injected into rabbits. Rabbit IgG polyclonal antibodies can purified, for example, on a peptide affinity column. The antibodies can then be used to bind to and identify the polypeptides of the invention that have been extracted and separated via gel electrophoresis or other means.

More recently, specialized PCR technologies have been applied to the problem of directed evolution (Stemmer, 1994, Proc. Natl. Acad. Sci. 91: 10747-51). The most popular version, primerless PCR or so-called sexual PCR, allows for the re-assortment, or “shuffling”, of closely related sequences. Briefly, a set of related gene sequences are fragmented, denatured, allowed to reanneal, and PCR extension is then performed through a number of cycles to reconstruct unit length genes. This process produces novel sequences that are complex permutations of the substrates. This process has proven to produce genes with significantly varied characteristics, and in many instances phenotypes dramatically improved for selected properties (e.g., Chang et al., 1999, Nat. Biotechnol. 8:793-7).

5.3 Starch Granules

The invention encompasses methods of altering the sizes of starch granules, the distribution of the sizes of starch granules, and/or the quantity of starch granules in a plant and the resulting modified starch produced.

In the context of the present invention, “altering the sizes of starch granules” means altering the dimensions, i.e. diameter or shape, of starch granules in the plant, by inhibiting or enhancing an FtsZ protein which effects aspects of starch granule growth limitations, such that starch granule sizes differ from the native plant. In the invention, this is achieved by altering the activity of the FtsZ product, which includes, but is not limited to, its function in plastid division, its temporal and spatial distribution and specificity, and its effect on starch granule growth limitations. The effects of altering the activity of the FtsZ may include, for example, increasing or decreasing the starch yield of the plant; increasing or decreasing the sizes of starch granules; altering temporal or spatial aspects of starch production or granule sizes in the plant; altering the distribution of starch granule sizes; and altering the type of starch produced. For example, the endosperm of mature wheat and barley grains contain two major classes of starch granules: large, early formed “A” granules and small, later formed “B” granules. Type A starch granules in wheat are about 20 μm diameter and type B around 5 μm in diameter (Tester, 1997, in: Starch Structure and Functionality, Frazier et al., eds., Royal Society of Chemistry, Cambridge, UK). Type A starch granules can also be considered greater than 10 um in diameter, while type B granules can be considered less than 10 um in diameter. The value defining the division between larger and small granules can vary depending on the genetic background of plant or the species of plant studied. In one embodiment the defining value is 6, 7, 8, 9, 10, 11, 12, 13, 14, 15, 16, 17, 18, 19, 20, 21, 22, 23, 24, 25, 26, 27, 28, 29, 30, 31, 32, 33, 34,,35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 um in diameter.

The quality of starch in wheat and barley is greatly influenced by the ratio of A-granules to B-granules. Altering the activity of the FtsZ protein will influence the limitations of sizes of starch granules, which is an important factor in determining the number and size of formed starch granules. The degree to which the FtsZ activity of the plant is affected will depend at least upon the nature and of the nucleic acid molecule or antagonist introduced into the plant, and the amount present. By altering these variables, the degree to which the sizes of starch granules can be regulated, the distribution of starch granule sizes, and/or the quantity of starch granules is manipulated according to the desired end result.

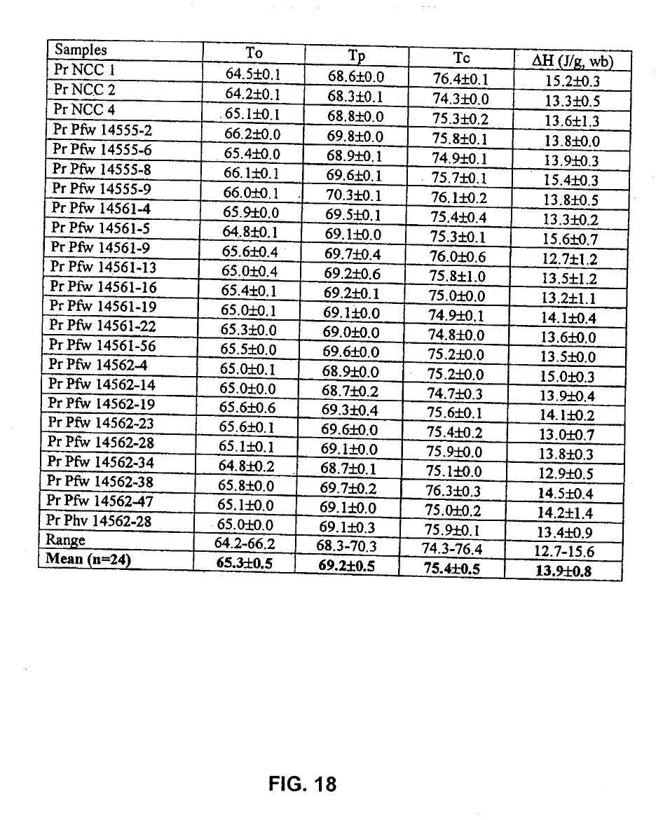

The methods of the invention (i.e. engineering-a plant to express a construct comprising a FtsZ nucleic acid) can, in addition to altering the sizes, distribution, and quantity of starch granules, alter the fine structure of starch in several ways including but not limited to, altering the ratio of amylose to amylopectin. The alteration in the sizes, distribution, and quantity of starch granules can in turn affect the functional characteristics of starch. The invention provides for a method of altering one or more starch characteristics comprising growing a plant comprising an FtsZ nucleic acid, such that the overall size of the starch granules is altered relative to that of a plant without the nucleic acid, wherein the characteristics of the starch from the plant with the nucleic acid is modified relative to a plant without the nucleic acid. The starch characteristics that can be altered by the methods of the invention include but are not limited to viscosity, elasticity, altered DSC values, gelling, thickness, foam density, pasting, or rheological properties of the starch such as those measured using viscometric analysis (FIG. 18). The modified starch can also be characterized by an alteration of more than one of the above-mentioned properties.

In particular, the engineered plants of the invention that produce starch consisting of starch granules with increased size, as measured by granule diameter, will exhibit greater ease of extractability. Starch extraction may be achieved by means common in the art, for example enzyme extraction, or mechanical means for disintegrating starch-containing plant tissues, washing out starch from the tissues and separating the starch granules from the by-products. Separating can be achieved by forcing the plant material through a series of rotary screens in a counter current process while continuously removing by-products with washes of water. Foaming techniques for starch extraction are also popular for some applications. For example, potato processing include hydraulic water washing, this water circulates at high speed, in short circuits. As the water is re-used several times, its content in organic components coming from the potatoes (proteins, starch, and solid particles) increases during the production time. All those ingredients combined form a light foam, rapidly growing, especially when the speed of the water is high. Various commercially available defoamers can then be applied in powder or liquid form throughout the process to extract particular components from the foam and evaporate the water.

In an embodiment of the invention, the size of starch granules is increased or decreased by at least 5%, 10%, 15%, 20%, 30%, 40%, 50%, 60%, 70%, 80%, 90% or more in comparison to a non-engineered control plant(s).

In the context of the present invention, alteration of the “distribution of sizes of starch granules” means, the sizes of all the starch granules in a sample correlated to the quantity or frequency of granules present for each size of granules. The distribution can comprise a single peak of frequency of granule sizes as is the case with potato, or two peaks as with barley, or more than two peaks. Alterations in the distribution can include, but are not limited to shifts of the peak towards larger sizes of granules, shifts in the peak towards smaller sizes of granules, a decrease of the height of the peak, i.e. a decrease in the frequency or quantity of the most common granule sizes, or combinations of these alterations, wherein two peaks are observed to be altered in different manners in a distribution, in comparison to a distribution of starch granules found in a non-engineered control plant(s).

In an embodiment of the invention, the ratio of amylose to amylopectin increases by 10%, 20%, 30%, 40%, or 50% in comparison to a non-engineered control plant(s). Plants engineered to express the nucleic acids of the invention to produce an increase in the sizes of starch granules as described herein, will result in an increase in the ratio because the outer growth layers of larger sized starch granules typically contain greater quantities of amylose than amylopectin.

In an embodiment of the invention, the ratio of amylose to amylopectin decreases by 10%, 20%, 30%, 40%, or 50% in comparison to a non-engineered control plant(s). Plants engineered to express the nucleic acids of the invention to produce an decrease in the sizes of starch granules as described herein, will result in an decrease in the ratio because the outer growth layers of larger sized starch granules typically contain greater quantities of amylose than amylopectin.

According to one aspect of the invention, the ratio of small starch granules to large granules is altered, i.e. increased or decreased, by at least 10%, 20%, 30%, 40%, 50%, 60%, 70%, 80%, 90% or more in comparison to a non-engineered control plant(s). 25 The invention provides for altering the sizes of starch granules wherein at least one of the starch granules is larger than any of the granules found in a plant without the nucleic acid molecule. In this embodiment, the large starch granule may be larger in diameter/dimension than native starch granules by 5 um, 10 um, 15, um, 20 um, 25 um, 30 um, 35 um, 40 um, 45 um, 50 um, 55 um, or 60 um. In one embodiment, the starch granules are as large in diameter/dimension as the largest native starch granules, but occur at an increased frequency.

The modified starch of the invention can be further modified by traditional means such as cross-linking, oxidizing, or conversion (Wurzburg, 1986, Modified starches: properties and uses, CRC Press, Boca Raton, Fla.)

5.4 Production of Transgenic Plants and Plant Cells

The invention also encompasses transgenic or genetically-engineered plants, and progeny thereof. As used herein, a transgenic or genetically-engineered plant refers to a plant and a portion of its progeny which comprises a nucleic acid molecule which is not native to the initial parent plant. The introduced nucleic acid molecule may originate from the same species e.g., if the desired result is over-expression of the endogenous gene, or from a different species. A transgenic or genetically-engineered plant may be easily identified by a person skilled in the art by comparing the genetic material from a non-transformed plant, and a plant produced by a method of the present invention for example, a transgenic plant may comprise multiple copies of FtsZ genes, and/or foreign nucleic acid molecules. Transgenic plants are readily distinguishable from non-transgenic plants by standard techniques. For example a PCR test may be used to demonstrate the presence or absence of introduced genetic material. Transgenic plants may also be distinguished from non-transgenic plants at the DNA level by Southern blot or at the RNA level by Northern blot or at the protein level by western blot, by measurement of enzyme activity or by starch composition or properties.

The nucleic acids of the invention may be introduced into a cell by any suitable means. Preferred means include use of a disarmed Ti-plasmid vector carried by Agrobacterium by procedures known in the art, for example as described in EP-A-01 16718 and EP-A-0270822. Agrobacterium mediated transformation methods are now available for monocots, for example as described in EP 0672752 and WO00/63398. Alternatively, the nucleic acid may be introduced directly into plant cells using a particle gun. A further method would be to transform a plant protoplast, which involves first removing the cell wall and introducing the nucleic acid molecule and then reforming the cell wall. The transformed cell can then be grown into a plant.

In an embodiment of the present invention, Agrobacterium is employed to introduce the gene constructs into plants. Such transformations preferably use binary Agrobacterium T-DNA vectors (Bevan, 1984, Nuc. Acid Res. 12:8711-21), and the co-cultivation procedure (Horsch et al., 1985, Science 227:1229-31). Generally, the Agrobacterium transformation system is used to engineer dicotyledonous plants (Bevan et al., 1982, Ann. Rev. Genet. 16:357-84; Rogers et al., 1986, Methods Enzymol. 118:627-41). The Agrobacterium transformation system may also be used to transform, as well as transfer, DNA to monocotyledonous plants and plant cells (see Hernalsteen et al., 1984, EMBO J. 3:3039-41; Hooykass-Van Slogteren et al., 1984, Nature 311:763-4; Grimsley et al., 1987, Nature 325:1677-79; Boulton et al., 1989, Plant Mol. Biol. 12:31-40.; Gould et al., 1991, Plant Physiol. 95:426-34). Wheat transformed with Agrobacterium using the seed inoculation method described in WO 00/63398 (RhoBio S. A.) can also be used.

Various alternative methods for introducing recombinant nucleic acid constructs into plants and plant cells may also be utilized. These other methods are particularly useful where the target is a monocotyledonous plant or plant cell. Alternative gene transfer and transformation methods include, but are not limited to, protoplast transformation through calcium-, polyethylene glycol (PEG)-or electroporation-mediated uptake of naked DNA (see Paszkowski et al., 1984, EMBO J. 3:2717-22; Potrykus et al., 1985, Mol. Gen. Genet. 199:169-177; Fromm et al., 1985, Proc. Natl. Acad. Sci. USA 82:5824-8; Shimamoto, 1989, Nature 338:274-6), and electroporation of plant tissues (D'Halluin et al., 1992, Plant Cell 4:1495-1505). Additional methods for plant cell transformation include microinjection, silicon carbide mediated DNA uptake (Kaeppler et al., 1990, Plant Cell Reporter 9:415-8), and microprojectile bombardment (Klein et al., 1988, Proc. Natl. Acad. Sci. USA 85:4305-9; Gordon-Kamm et al., 1990, Plant Cell 2:603-18).

According to the present invention, desired plants and plant cells may be obtained by engineering the gene constructs described herein into a variety of plant cell types, including, but not limited to, protoplasts, tissue culture cells, tissue and organ explants, pollen, embryos as well as whole plants. In an embodiment of the present invention, the engineered plant material is selected or screened for transformants (i.e., those that have incorporated or integrated the introduced gene construct or constructs) following the approaches and methods described below. An isolated transformant may then be regenerated into a plant. Alternatively, the engineered plant material may be regenerated into a plant, or plantlet, before subjecting the derived plant, or plantlet, to selection or screening for the marker gene traits. Procedures for regenerating plants from plant cells, tissues or organs, either before or after selecting or screening for marker gene or genes, are well known to those skilled in the art.

A transformed plant cell, callus, tissue or plant may be identified and isolated by selecting or screening the engineered plant material for traits encoded by the marker genes present on the transforming DNA. For instance, selection may be performed by growing the engineered plant material on media containing inhibitory amounts of the antibiotic or herbicide to which the transforming marker gene construct confers resistance. Further, transformed plants and plant cells may also be identified by screening for the activities of any visible marker genes (e.g., the β-glucuronidase, luciferase, green fluorescent protein, B or C1 anythocyanin genes) that may be present on the recombinant nucleic acid constructs of the present invention. Such selection and screening methodologies are well known to those skilled in the art.

The present invention is applicable to all plants which produce or store starch. Examples of such plants are cereals such as maize, wheat, rice, sorghum, barley; fruit producing species such as banana, apple, tomato or pear; root crops such as cassava, potato, yam, beet or turnip; oilseed crops such as rapeseed, canola, sunflower, oil palm, coconut, linseed or groundnut; meal crops such as soya, bean or pea; and any other suitable species. Suitable plants can be monocots, dicots, gymnosperms, annuals, perennial, herbaceous, trees or other woody plants.

In a preferred embodiment of the present invention, the method comprises the additional step of growing the plant and harvesting the starch from a plant part. In order to harvest the starch, it is preferred that the plant is grown until plant parts containing starch develop, which may then be removed. In a further preferred embodiment, the propagating material from the plant may be removed, for example the seeds. The plant part can be an organ such as a stem, root, leaf, or reproductive body. Alternatively, the plant part may be a modified organ such as a tuber, or the plant part is a tissue such as seed or seed endosperm.

5.5 Transgenic Plants that Express Plant FtsZ

The present invention provides a method for producing plants with altered number and/or size of starch granules by manipulating the division of amyloplasts. Amyloplast division, and hence starch granule number and/or size, may be altered by augmenting or by disrupting the expression of the endogenous gene or genes involved in amyloplast division. The former may be achieved by over expression of the introduced nucleotide sequence comprising a native or heterologous FtsZ gene, e.g. increasing the copy number of the introduced sequence such that more FtsZ is produced. The latter may be achieved, for example, by antisense down regulation, or by co-suppression (e.g. by introduction of partial sense sequences), or by double stranded RNA technology (also known as duplex technology), all techniques well known in the art. Additionally, dual constructs may be expressed in a single plant. For example an FtsZ1 gene or fragment thereof and an FtsZ2 gene or fragment thereof can both be expressed in a single plant to alter the sizes of starch granules and/or the distribution of sizes of starch granules or the quantity of starch.

In less preferred embodiments, the nucleic acid molecules used in producing transgenic plants are not FtsZ genes from Arabidopsis. In yet other less preferred embodiments, the nucleic acid molecules used in producing transgenic plants are not FtsZ genes from tobacco, rice, maize, pea and/or wheat.

A plant that expresses a recombinant FtsZ nucleic acid may be engineered by transforming a plant cell with a nucleic acid construct comprising a regulatory region operably associated with a nucleic acid molecule oriented in a sense direction, the sequence of which encodes a FtsZ protein or a fragment thereof. In plants derived from such cells, starch granules are altered. In one embodiment, the FtsZ nucleic acid molecule oriented in a sense direction comprises the sequence of SEQ ID NO: 1, 3, 5, 7, 9, 11, 13, or 15, or a fragment or variant thereof.

The term “operably associated” is used herein to mean that transcription controlled by the associated regulatory region would produce a functional mRNA. Starch may be altered in particular parts of a plant, including but not limited to leaves, seeds, tubers, leaves, roots and stems or modifications thereof.

In one embodiment of the present invention, desired plants with suppressed target gene expression may be engineered by transforming a plant cell with a co-suppression construct. A co-suppression construct comprises a functional promoter operatively associated with a full length or partial FtsZ nucleic acid sequence. According to the present invention, it is preferred that the co-suppression construct encodes FtsZ gene mRNA or enzyme, although a construct encoding an incomplete FtsZ gene mRNA may also be useful in effecting co-suppression. Examples of such constructs can be found in section 6. In one embodiment, the nucleic acids of the invention are fragments of an FtsZ gene that are expressed as RNA under conditions that facilitate co-suppression of one or more FtsZ genes. Fragments of the sequences of the invention may be expressed in a sense orientation to achieve a co-suppression effect, i.e. fewer starch granules that are larger, while the full length cDNAs can be expressed in a sense orientation to overexpress the nucleic acid, i.e. increase the number and decrease the size of starch granules. Alterations in starch and starch granules that can be achieved by the methods of the invention are further disclosed in ways described in section 5.3, 5.4, 5.5, and 5.6. Fragments of the sequences of the invention may be expressed in a bacteria, yeast, algae, fungi, plant, or animal cell.

In another embodiment of the invention, the nucleic acid molecule expressed in the plant cell, plant, or part of a plant comprises a recombinant nucleotide sequence encoding a plant FtsZ protein, or variant thereof. The nucleic acid molecule expressed in the plant cell can comprise a nucleotide sequence encoding a full length FtsZ protein. Examples of such sequences include SEQ ID NOs: 12 or 14, or variants thereof and nucleotide sequences that encode the amino acid sequences of SEQ ID NOs: 11 or 13 or variants thereof. In a related embodiment, the recombinant nucleic acid molecule expressed in the plant cell consists essentially of a full length FtsZ cDNA and functions in the methods of the invention as a full length sequence. Sense directed expression or overexpression of full length FtsZ genes in plants can decrease the sizes of starch granules and/or shift the distribution of sizes of starch granules towards smaller granules or alter the quantity of starch.

Sense directed co-suppression of full length FtsZ genes in plants can increase the sizes of starch granules and/or shift the distribution of sizes of starch granules towards larger granules or alter the quantity of starch.

In yet another embodiment of the invention, the starch content of plants and cells engineered to express the nucleic acids of the invention, the quantity of starch granules, the sizes of starch granules, and/or the distribution of sizes of starch granules of the plant cell and plants derived from such cells exhibit altered characteristics. The altered starch content comprises an alteration in the ratio of amylose to amylopectin. In specific embodiments of the invention, where FtsZ protein activity is decreased by co-suppression of native FtsZ expression, the ratio of amylose to amylopectin increases by 2%, 5%, 10%, 20%, 30%, 40%, or 50% in comparison to a non-engineered control plant(s). In a preferred embodiment, the ratio of amylose to amylopectin increases by 5%-20%.

In various embodiments, a plant genetically-engineered with the nucleic acid molecules of the invention exhibits an altered quantity of starch granules, wherein the quantity increases or decreases by 2%, 5%, 10%, 20%.

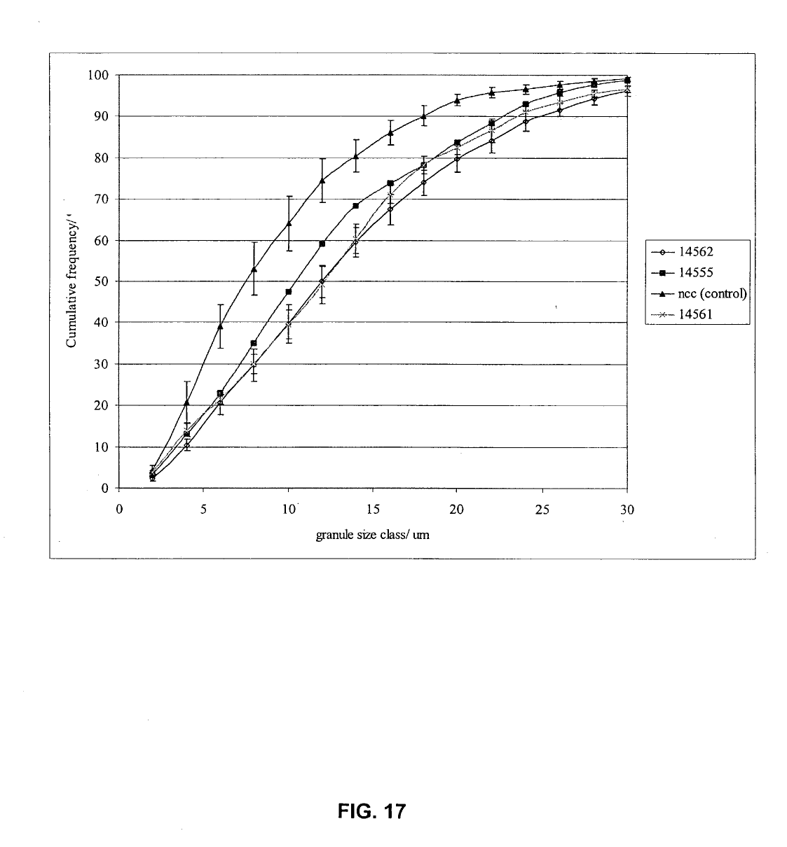

In a preferred embodiments, a genetically-engineered potato plant comprises a patatin promoter operably linked to a nucleic acid molecule of SEQ ID NO: 1 or 9, such that said patatin promoter regulates transcription of the nucleic acid molecule, and the sizes of starch granules in the plant are altered relative to a potato plant not comprising the nucleic acid molecule, such that the sizes of starch granules are more uniform. For example, in FIG. 17, the frequency of classes of sizes of starch granules between 8 and 20 urn in diameter decreases in the transgenic plant lines (14562 with SEQ ID NO: 9 in antisense direction; 14555 with SEQ ID NO: 1 in the sense direction; and 14561 with SEQ ID NO: 9 in the sense direction) in comparison to the non-transgenic plant lines (ncc or control). The amount of observed decrease is greater than the amount of decrease in the frequency of classes of sizes of starch granules less than 8 urn and classes of sizes greater than 20 um. Thus, the distribution of sizes of starch granules in the transgenic lines is more uniform in comparison to the distribution of sizes of granules in the non-transgenic control plants. The invention also provides for starch extracted from such a plant. The distribution of sizes of starch granules in non-transgenic control potato plants comprises a single peak of starch granules between 8 and 20 um in diameter. The distribution of sizes of starch granules in potato plants expressing the nucleic acids of the invention, as described above and exemplified in FIG. 17, exhibits a widening or flattening of the distribution peak, such that the sizes of starch granules exhibit a more uniform distribution. In one embodiment, the peak of the distribution of starch granules shifts towards larger granule size by 2 um, 5 um, 10 um, 15 um, or 20 um. In a preferred embodiment, the peak of the distribution of starch granules shifts towards larger granule size by 10 um.

In a preferred embodiment, a genetically-engineered barley plant comprises a HMWG promoter operably linked to a wheat nucleic acid molecule of SEQ ID NO: 5 in an sense orientation, such that said HMWG promoter regulates transcription of the nucleic acid molecule, and the sizes of starch granules in the plant are altered relative to a barley plant not comprising the nucleic acid molecule, resulting in altered ratios of large to small granules. In one embodiment, the ratio increases by 2%, 5%, 10%, 15%, 20%, 25%, 30%, 35%, 40%, 45%, or 50% or more. In a preferred embodiment, the ratio increases by 5%-25%.

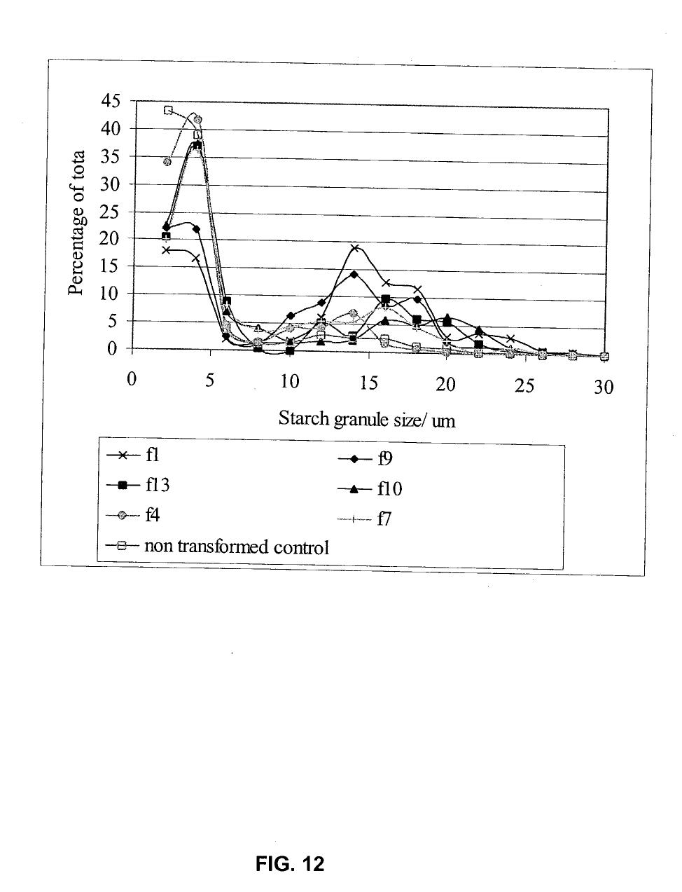

In another prefered embodiment, a genetically-engineered cereal plant comprises a HMWG promoter operably linked to a nucleic acid molecule of SEQ ID NO: 5 in an sense orientation, such that said HMWG promoter regulates transcription of the nucleic acid molecule, and the sizes of starch granules in the plant exhibit an increase in a ratio of large to small granules relative to a cereal plant not comprising the nucleic acid molecule, wherein small granules are less than or equal toIO um in diameter and large granules are greater than 10 um in diameter. For example, FIG. 12 shows control barley plants compared to barley plants genetically-engineered to express the nucleic acid sequence of SED ID NO: 5 in an sense orientation. The increase in the ratio of large to small granules observed can be the result of a decrease in small granules and an increase in large granules as is the case with the f1 and f9 transgenic lines in FIG. 12. The increase in the ratio of large to small granules observed can also be the result of an increase in the large granules as is the case with transgenic line f13. In one embodiment, the ratio increases by 2%, 5%, 10%, 15%, 20%, 25%, 30%, 35%, 40%, 45%, or 50% or more. In a preferred embodiment, the ratio increases by 5%-25%. In this embodiment the cereal plant can be maize, wheat, barley, rye, or progeny or a hybrid plant thereof. The invention also provides for starch extracted from such a plant or progeny thereof which plant contains the nucleic acid molecule.

In a preferred embodiment, a genetically-engineered barley plant comprises a HMWG promoter operably linked to a wheat nucleic acid molecule of SEQ ID NO: 5 in an sense orientation, such that said HMWG promoter regulates transcription of the nucleic acid molecule, and the sizes of starch granules in the plant are altered relative to a barley plant not comprising the nucleic acid molecule, resulting in altered ratios of large to small granules. In one embodiment, the ratio decreases by 2%, 5%, 10%, 15%, 20%, 25%, 30%, 35%, 40%, 45%, or 50% or more. In a preferred embodiment, the ratio decreases by 5%-25%.

In a preferred embodiment, a genetically-engineered potato plant comprises a patatin promoter operably linked to a nucleic acid molecule of SEQ ID NO: 9 in an sense orientation, such that said patatin promoter regulates transcription of the nucleic acid molecule, and the sizes of starch granules in the plant are altered relative to a potato plant not comprising the nucleic acid molecule, resulting in starch granules more uniform in size as described above in relation to FIG. 17. The invention also provides for starch extracted from such a plant or progeny plants thereof, which plants have the nucleic acid molecule.

In preferred embodiments of the invention, the cereal plants transformed with the nucleic acids of the invention can be maize, wheat, barley, rye, or progeny or a hybrid plant thereof. The invention also provides for starch extracted from such a plant or progeny thereof which plant contains the nucleic acid molecule.

In preferred embodiment of the invention, the nucleic acid molecules of the invention are expressed in a potato plant and are transcribed only in the sense orientation. The starch content of the plant, including the tubers, exhibit a modulation in the quantity of starch granules, an alteration in the sizes of starch granules, and/or distribution of sizes of starch granules. If a number of copies of the FtsZ nucleic acid molecules of the invention are expressed in a potato plant in the sense orientation, the effect on the quantity of starch granules, an alteration in the sizes of starch granules, and/or distribution of sizes of starch granules is amplified with greater copy number.

In yet another embodiment of the present invention, it may be advantageous to transform a plant with a nucleic acid construct operably linking a modified or artificial promoter to a nucleic acid molecule having a sequence encoding a FtsZ protein or a fragment thereof. Such promoters typically have unique expression patterns and/or expression levels not found in natural promoters because they are constructed by recombining structural elements from different promoters. See, Salina et al., 1992, Plant Cell 4:1485-93, for examples of artificial promoters constructed from combining cis-regulatory elements with a promoter core.

In one embodiment of the present invention, the associated promoter is a strong leaf, stem, root and/or embryo-specific plant promoter such that the FtsZ protein is overexpressed in the transgenic plant.

In yet another preferred embodiment of the present invention, the overexpression of FtsZ protein in starch producing organs and organelles may be engineered by increasing the copy number of the FtsZ gene. One approach to producing such transgenic plants is to transform with nucleic acid constructs that contain multiple copies of the complete FtsZ nucleic acid with native or heterolgous promoters. Another approach is repeatedly transform successive generations of a plant line with one or more copies of the complete FtsZ nucleic acid constructs. Yet another approach is to place a complete FtsZ gene in a nucleic acid construct containing an amplification-selectable marker (ASM) gene such as the glutamine synthetase or dihydrofolate reductase gene. Cells transformed with such constructs is subjected to culturing regimes that select cell lines with increased copies of complete FtsZ gene. See, e.g., Donn et al., 1984, J. Mol. Appl. Genet. 2:549-62, for a selection protocol used to isolate of a plant cell line containing amplified copies of the GS gene. Cell lines with amplified copies of an FtsZ nucleic acid can then be regenerated into transgenic plants.

In another embodiment of the invention, the method further comprises introducing into the plant a nucleotide sequence comprising a plant glycogenin-like gene or starch primer gene, or a fragment thereof. In the context of the present invention, a “plant glycogenin-like protein” or “starch primer” includes any protein which is capable of initiating starch production in a plant (Great Britain Patent Application No. 0119342.4 PCT/GB2002/003636) By definition, the plant glycogenin-like protein will typically be native to a plant. Preferred fragments thereof are those which retain the ability to initiate starch synthesis. An advantage of this embodiment is that it creates the possibility to manipulate the number and/or size of starch granules by affecting both the initiation of starch granules, via the nucleotide sequence comprising a plant glycogenin-like gene, and the subsequent development of the starch granules via the nucleotide sequence comprising an FtsZ gene.

5.6 Antisense Down Regulation of Endogenous Plant FtsZ

The nucleic acid molecules of the invention can also be used to alternatively alter activity of the FtsZ protein of a plant cell, plant, or part of a plant by modifying transcription or translation of the FtsZ nucleic acid. In an embodiment of the invention, an antagonist which is capable of altering the expression of a nucleic acid molecule of the invention or a native FtsZ gene product is introduced into a plant in order to alter the size, number and distribution of starch granules. The antagonist may be protein, nucleic acid, chemical antagonist, or any other suitable moiety. In an embodiment of the invention, an antagonist which is capable of altering the expression of a nucleic acid molecule of the invention is provided to alter the synthesis of starch. The antagonist may be protein, nucleic acid, chemical antagonist, or any other suitable moiety. Typically, the antagonist will function by inhibiting or enhancing transcription from the FtsZ nucleic acid, either by affecting regulation of the promoter or the transcription process; inhibiting or enhancing translation of any RNA product of the FtsZ nucleic acids; inhibiting or enhancing the activity of the FtsZ protein itself or inhibiting or enhancing the protein-protein interaction of the FtsZ protein and growth and size formation of starch granules. For example, where the antagonist is a protein it may interfere with transcription factors binding to the FtsZ gene promoter, mimic the activity of a transcription factor, compete with or mimic the FtsZ protein, or interfere with translation of the FtsZ RNA, interfere with the interaction of the FtsZ protein and downstream enzymes. Antagonists which are nucleic acids may encode proteins described above, or may be transposons which interfere with expression of the FtsZ nucleic acids. Examples of suitable antisense DNAs are the antisense DNAs of the sequences shown in SEQ ID NOS: 1, 3, 5, 7, 9, 11, 13, 15, 17, 19, or 21.

Full length FtsZ sequences of the invention can also be used in antisense constructs. Examples of such sequences include SEQ ID NOs: 12 or 14, or variants thereof and nucleotide sequences that encode the amino acid sequences of SEQ ID NOs: 11 or 13 or variants thereof. Antisense directed expression or overexpression of full length FtsZ genes in plants can increase the sizes of starch granules and/or shift the distribution of sizes of starch granules towards larger granules or alter the quantity of starch. In a related embodiment the nucleic acid of the invention consists essentially of a full length FtsZ cDNA and functions in the methods of the invention as a full length sequence.

Full length sequences of the invention and fragments thereof may be expressed in an antisense orientation in bacteria, yeast, algae, fungi, plant, or animal cell.

In a preferred embodiment, a genetically-engineered potato plant comprises a patatin promoter operably linked to a nucleic acid molecule of SEQ ID NO: 9 in an sense antisense orientation, such that said patatin promoter regulates transcription of the nucleic acid molecule, and the sizes of starch granules in the plant are altered relative to a potato plant not comprising the nucleic acid molecule, resulting in starch granules more uniform in size as described above in relation to FIG. 17. The invention also provides for starch extracted from such a plant or progeny plants thereof, which plants have the nucleic acid molecule.

In another prefered embodiment, a genetically-engineered cereal plant comprises a HMWG promoter operably linked to a nucleic acid molecule of SEQ ID NO: 5 in an antisense orientation, such that said HMWG promoter regulates transcription of the nucleic acid molecule, and the sizes of starch granules in the plant exhibit an increase in a ratio of large to small granules relative to a cereal plant not comprising the nucleic acid molecule, wherein small granules are less than or equal to 10 um in diameter and large granules are greater than 10 um in diameter. In this embodiment the cereal plant can be maize, wheat, barley, rye, or progeny or a hybrid plant thereof. The invention also provides for starch extracted from such a plant or progeny thereof which plant contains the nucleic acid molecule.

The suppression may be engineered by transforming a plant with a nucleic acid construct encoding an antisense RNA or ribozyme complementary to a segment or the whole of FtsZ gene RNA transcript, including the mature target mRNA. In another embodiment, FtsZ gene suppression may be engineered by transforming a plant cell with a nucleic acid construct encoding a ribozyme that cleaves the FtsZ gene mRNA transcript.

In another embodiment, the FtsZ mRNA transcript can be suppressed through the use of RNA interference, referred to herein as RNAi. RNAi allows for selective knock out of a target gene in a highly effective and specific manner. The RNAi technique involves introducing into a cell double-stranded RNA (dsRNA) which corresponds to exon portions of a target gene such as an endogenous FtsZ gene. The dsRNA causes the rapid destruction of the target gene's messenger RNA, i.e. an endogenous FtsZ gene mRNA, thus preventing the production of the FtsZ protein encoded by that gene. The RNAi constructs of the invention confer expression of dsRNA which correspond to exon portions of an endogenous FtsZ gene. The strands of RNA that form the dsRNA are complementary strands from coding region of the FtsZ gene. Preferably the strands are from the 3′ end of the FtsZ gene.

The dsRNA has an effect on the stability of the mRNA. The mechanism of how dsRNA results in the loss of the targeted homologous mRNA is still not well understood (Cogoni and Macino, 2000, Genes Dev 10: 638-643; Guru, 2000, Nature 404, 804-808; Hammond et al., 2001, Nature Rev Gen 2: 110-119). Current theories suggest a catalytic or amplification process occurs that involves initiation step and an effector step.

In the initiation step, input dsRNA is digested into 21-23 nucleotide “guide RNAs”. These guide RNAs are also referred to as siRNAs, or short interfering RNAs. Evidence indicates that siRNAs are produced when a nuclease complex, which recognizes the 3′ ends of dsRNA, cleaves dsRNA (introduced directly or via a transgene or virus) ˜22 nucleotides from the 3′ end. Successive cleavage events, either by one complex or several complexes, degrade the RNA to 19-20 bp duplexes (siRNAs), each with 2-nucleotide 3′ overhangs. RNase III-type endonucleases cleave dsRNA to produce dsRNA fragments with 2-nucleotide 3′ tails, thus an RNase III-like activity appears to be involved in the RNAi mechanism. Because of the potency of RNAi in some organisms, it has been proposed that siRNAs are replicated by an RNA-dependent RNA polymerase (Hammond et al., 2001, Nature Rev Gen 2:110-119; Sharp, 2001, Genes Dev 15: 485-490).

In the effector step, the siRNA duplexes bind to a nuclease complex to form what is known as the RNA-induced silencing complex, or RISC. The nuclease complex responsible for digestion of mRNA may be identical to the nuclease activity that processes input dsRNA to siRNAs, although its identity is currently unclear. In either case, the RISC targets the homologous transcript by base pairing interactions between one of the siRNA strands and the endogenous mRNA. It then cleaves the mRNA ˜12 nucleotides from the 3′ terminus of the siRNA (Hammond et al., 2001, Nature Rev Gen 2:110-119; Sharp, 2001, Genes Dev 15:485-490).

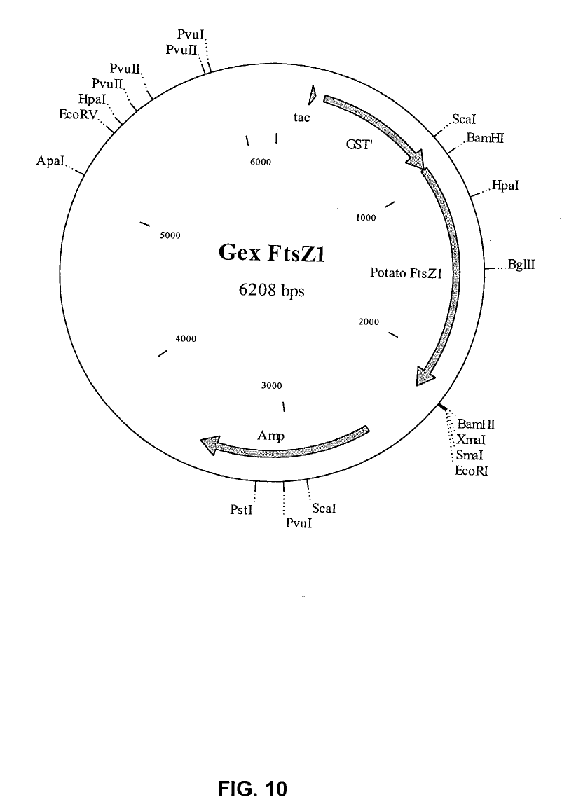

Methods and procedures for successful use of RNAi technology in post-transcriptional gene silencing in plant systems has been described by Waterhouse et al. (Waterhouse et al., 1998, Proc Natl Acad Sci USA, 95(23):13959-64). Methods specific to construction of the RNAi constructs of the invention can be found in Examples 2 and 6 as well as FIGS. 6 and 10. While the invention encompasses use of any FtsZ gene of the invention in the RNAi constructs, in a preferred embodiment, the strands of RNA that form the dsRNA are complementary strands encoded by a coding region on the 3′ end of an FtsZ gene sequence.

For all of the aforementioned constructs, it is preferred that such nucleic acid constructs express specifically in organs where starch synthesis occurs (i.e. tubers, seeds, stems roots and leaves) and/or the plastids where starch synthesis occurs. Alternatively, it may be preferred to have the suppression or antisense constructs expressed constitutively. Thus, constitutive promoters, such as the nopaline, CaMV 35S promoter, may also be used to express the suppression constructs. A most preferred promoter for these suppression or antisense constructs in cereals is a rice actin promoter. Alternatively, a co-suppression construct promoter can be one that expresses with the same tissue and developmental specificity as the FtsZ gene.

In accordance with the present invention, desired plants with suppressed target gene expression may also be engineered by transforming a plant cell with a construct that can effect site-directed mutagenesis of the FtsZ nucleic acid molecules. For discussions of nucleic acid constructs for effecting site-directed mutagenesis of target genes in plants see, e.g., Mengiste et al., 1999, Biol. Chem. 380:749-758; Offringa et al., 1990, EMBO J. 9:3077-84; and Kanevskii et al., 1990, Dokl. Akad. Nauk. SSSR 312:1505-7. It is preferred that such constructs effect suppression of FtsZ genes by replacing the endogenous FtsZ gene nucleic acid molecule through homologous recombination with either an inactive or deleted FtsZ protein coding nucleic acid molecule.

In yet another embodiment, antisense technology can be used to inhibit FtsZ gene mRNA expression. Alternatively, the plant can be engineered, e.g., via targeted homologous recombination to inactive or “knock-out” expression of the plant's endogenous FtsZ protein. The plant can be engineered to express an antagonist that hybridizes to one or more regulatory elements of the gene to interfere with control of the gene, such as binding of transcription factors, or disrupting protein-protein interaction. The plant can also be engineered to express a co-suppression construct. The suppression technology may also be useful in down-regulating the native FtsZ gene of a plant where a foreign FtsZ nucleic acid has been introduced. To be effective in altering the activity of a FtsZ protein in a plant, it is preferred that the nucleic acid molecules are at least 50, preferably at least 100 and more preferably at least 150 nucleotides in length. In one aspect of the invention, the nucleic acid molecule expressed in the plant cell can comprise a nucleotide sequence of the invention which encodes a full length FtsZ protein and wherein the nucleic acid molecule has been transcribed only in the antisense direction.

In another preferred embodiment, the sizes of starch granules and/or the distribution of sizes of starch granules from certain plant organs or tissues is altered in comparison to a non-engineered control plant(s). In other embodiments, the sizes of starch granules and/or the distribution of sizes of starch granules of tubers, or seeds is altered in plants engineered using the antisense technology described herein when compared to the starch content in a non-engineered control plant(s). Plant tissues in which the sizes of starch granules and/or the distribution of sizes of starch granules can be altered using the methods of the invention include but are not limited to endosperm, leaf mesophyll, and root or stem cortex or pith.

In another aspect of the invention, the nucleic acid molecules of the invention are expressed in a plant cell engineered expressing an FtsZ nucleic acids of the invention. The plant cell or cultures of cells can be used to regenerate plants expressing the FtsZ nucleic acids.

In one embodiment, the ratio of large starch granules to small starch granules increases in a cereal plant. An increased ratio of large starch granules to small starch granules results in greater accessibility of starch granules, which has certain industrial and commercial advantages related to extraction and processing of starch.

The progeny of the transgenic or genetically-engineered cells and plants of the invention containing the nucleic acids of the invention are also encompassed by the invention.

The embodiments described in each section above apply to the other aspects of the invention, mutatis mutandis.

### 6 EXAMPLES

### Example 1

Isolation of Potato FtsZ Type 2 cDNA Fragments.

Design of Degenerate Primers.

Full length sequences coding for both FtsZ type 1 and 2 from both monocotyledonous and dicotyledonous higher plant species and the moss Physcomitrella patens were identified from publicly available databases and analyzed. (Accession Numbers for the sequences were: 1. Arabidopsis thaliana; Q425445, AL353912 and AF089738. 2. Nicotiana tabacum; AJ271750, AJ133453, AJ271749, AJ271748 and AF205858. 3. Gentiana lutea; AF205859. 4. Pisum sativum; T06774. 5. Tagetes erecta; AF251346. 6. Lilium longiflorum; AB042101. 7. Physcomitrella patens; AJ001586 and AJ249139) Regions of these sequences having high homology at the protein level were identified and used to design a series of degenerate primers for use in PCR. The primers were tailed at the 5′ end with a 4 bp spacer and a BamHI restriction site (GGATCC) to enable the cloning of the fragments so generated into appropriate vectors. The sequences of these primers are shown below:

| | |
|---|---|
| FTSZ2FB: | |
| ACGTGGATCCAATGCKGTKAATMGKATGAT | (SEQ ID NO: 22) |
| FTSZ2RB: | |
| ACGTGGATCCGCKCCGAAKATKAKGTT | (SEQ ID NO: 23) |

It will be recognized by one skilled in the art that other PCR primers could be designed incorporating the features of FTSZ2FB and FTSZ2RB and alternative restriction enzyme sites.

cDNA Synthesis.

mRNA was extracted from leaf and tuber tissue of Solanum tuberosum c.v. Hermes according to the method given by Nucleon Biosciences in their plant RNA extraction kit. Double stranded cDNA was synthesized from these RNA samples using the procedure given in Clontech's SMARTTM PCR cDNA synthesis kit.

Isolation of FtsZ cDNA Fragments.

The cDNA preparations, produced as described above, were used as the template for isolation of a specific cDNA fragment of a potato FtsZ gene by PCR. PCR was carried out using the AdvanTAge 2 PCR kit from ClonTech The reactions contained 5 ul 10× Advantage Taq buffer; 5 ul 2mM dNTPs; 0.5 ul of primer FTSZ2FB (50 uM); 0.5 ul of primer FTSZ2RB (50 uM); 1 ul cDNA template; 1 ul Advantage Taq polymerase; 37 ul distilled water in a final volume of 50 ul. The PCR was carried out on a thermocycler using the following parameters:

| | | |
|---|---|---|
| Hot start: | 94° C. | 3 min |
| 15 cycles of: | | |
| Step 1 | 94° C. | 1 min |
| Step 2 | 55° C. | 1 min |
| Step 3 | 72° C. | 2 min |
| 15 cycles of: | | |
| Step 1 | 94° C. | 1 min |
| Step 2 | 60° C. | 1 min |
| Step 3 | 72° C. | 2 min |
| Followed by: | | |
| | 72° C. | 5 min |
| Hold at: | 8° C. | |

DNA fragments of about 800 bp were isolated. The fragments were purified by agarose gel electrophoresis and had A tails added to enable them to be inserted into the CloneTech TA cloning vector (pT-Adv) by incubating the fragment with 2 units Taq Polymerase and 0.2 mM dATP at 72° C. for 10 minutes. Ligation and transformation was carried out using the AdvanTAge PCR cloning kit from CLONTECH. A 50 ng aliquot of the vector was ligated with the cDNA fragment at 14° C. overnight. Chemically competent TOP10 E. coli cells were transformed with a 2 ul aliquot by heat shock and grown on selected media overnight. A combination of blue/white selection and colony PCR was used to select individual clones containing the advantage vector with inserted cDNA fragments. Individual colonies were grown up and plasmid DNA extracted for sequence analysis.

Sequence Analysis.

The FtsZ DNA fragments present in a number of independent pT-Adv clones were sequenced. Analysis of the sequence showed that all of the clones contained a fragment of the FtsZ gene family type designated as type 2. Further analysis revealed that there were two homologous but different sequences. These were designated potato FtsZ2a and potato FtsZ2b. They were represented in both the leaf and tuber cDNA preparations. The sequences of these fragments are shown in SEQ ID NOs: 1 and 3.

### Example 2

Isolation of Wheat FtsZ Type 2 cDNA Fragments.

cDNA library

A double stranded cDNA library was constructed from wheat mRNA extracted from seed at 18 days post anthesis using the SMARTTM PCR cDNA synthesis kit (CloneTech) as in Example 1.

Isolation of FtsZ cDNA Fragments.

The cDNA preparations, produced as described above, were used as the template for isolation of a specific cDNA fragment of a wheat FtsZ gene by PCR. PCR was carried out using the Advantage 2 PCR kit from CloneTech as described in Example 1.

DNA fragments of about 800 bp were isolated. The fragments were purified by agarose gel electrophoresis and had A tails added to enable them to be inserted into the CloneTech TA cloning vector (pT-Adv) by incubating the fragment with 2 units Taq Polymerase and 0.2 mM dATP at 72° C. for 10 minutes. Ligation mixtures were set up with a final volume of 10 μl containing 50 ng pT-Adv vector; 50 ng A tailed-PCR product; 1 μl ligase buffer, 10 mM DTT, 1 mM ATP and 0.5 ul T4 DNA ligase. Reactions were incubated at 14° C. for 16 hours. Ligation and transformation was carried out using the AdvanTAge PCR cloning kit from CLONTECH. A 50 ng aliquot of the vector was ligated with the cDNA fragment at 14° C. overnight. Chemically competent TOP10 E. coli cells were transformed with a 2 ul aliquot by heat shock and grown on selected media overnight. A combination of blue/white selection and colony PCR was used to select individual clones containing the pAdvantage vector with inserted cDNA fragments. Individual colonies were grown up and plasmid DNA extracted for sequence analysis.

Sequence Analysis

The FtsZ DNA fragments present in a number of independent pT-Adv clones were sequenced. Analysis of the sequence showed that all of the clones contained a fragment of the FtsZ gene family type designated as type 2. Further analysis revealed that there were two homologous but different sequences represented in the pT-Adv clones. These sequences were designated wheat FtsZ2a and wheat FtsZ2b. The sequences of these fragments are shown in SEQ ID NO: 5 and 7.

### Example 3

Isolation of Potato FtsZ Type 1 cDNA Fragments.

Design of FtsZ type 1 Specific Primers.

Because only type 2 sequences were obtained by PCR using degenerate primers designed using both FtsZ type 1 and FtsZ type 2 sequences, an alternative strategy was employed to obtain a potato FtsZ type 1 sequence. PCR primers were designed to the three Nicotiana tabacum sequences for FtsZ type 1 (NtFtsZ1-1, NtFtsZ1-2 and NtFtsZ1-3; Genbank accession numbers AJ272748, AJ133453 and AJ271749). The selected regions corresponded to regions of high homology at the protein level of all the previously listed type 1 sequences and in an equivalent region to the section used for the isolation of the FtsZ type 2 sequences. Two sets of primer pairs were designed and synthesized. The first set was specific for the N. tabacum cDNA sequences. The second set was based on the N. tabacum amino acid sequences with the necessary degeneracy factored in. The primers are listed below:

| | |
|---|---|
| Set 1. Tobacco specific. | |
| FZT1TOBF: | |
| TAGCGGATCCGTGGCAGTGGCTTGCAGGGTGTTGA | (SEQ ID NO: 24) |
| FZT1TOBR: | |
| ACTGGGATCCAKGGATCAGCCAGGCTKGTGACAA | (SEQ ID NO: 25) |
| Set 2. Degenerate. | |
| FZT1NEWR: | |
| ACTGGGATCCTGGATCMGCMAAMSWMGTMACM | (SEQ ID NO: 26) |
| FZT1NEWF: | |
| GCTAGGATCCGGKTTKCAGGGKGTKGATCCK | (SEQ ID NO: 27) |
| All primers contain a BamHI restriction enzyme | |
| digest site preceded by a 4 bp tail. | |
| cDNA synthesis. | |

mRNA was extracted from leaf and tuber tissue of Solanum tuberosum c.v. Hermes as described in Example 1. Double stranded cDNA was synthesized from these mRNA samples using the procedure given in Clontech's SMARTTM PCR cDNA synthesis kit as described for Example 1.

Isolation of FtsZ cDNA Fragments.

The cDNA preparations, produced as described above, were used as the template for isolation of a specific cDNA fragment of a potato FtsZ gene by PCR. PCR was carried out using the Advantage 2 PCR kit from CloneTech The reactions contained 5 ul 10× Advantage Taq buffer; 5 ul 2 mM dNTPs; 0.5 ul of primer FZT1TOBF (50 uM); 0.5 ul of primer FZT1TOBR (50 uM); 1 ul cDNA template; 1 ul Advantage Taq polymerase; 37 ul distilled water in a final volume of 50ul. Alternatively, the reactions contained 5 ul 10× Advantage Taq buffer; 5 ul 2 mM dNTPs; 0.5 ul of primer FZT1NEWR (50 uM); 0.5 ul of primer FZT1NEWF (50 uM); 1 ul cDNA template; 1 ul Advantage Taq polymerase; 37 ul distilled water in a final volume of 50 ul. The PCR, for either set of reaction mixtures, was carried out on a thermocycler using the following parameters:

| | | |
|---|---|---|
| Hot start: | 94° C. | 3 min |
| 15 cycles of: | | |
| Step 1 | 94° C. | 1 min |
| Step 2 | 55° C. | 1 min |
| Step 3 | 72° C. | 2 min |
| 15 cycles of: | | |
| Step 1 | 94° C. | 1 min |
| Step 2 | 60° C. | 1 min |
| Step 3 | 72° C. | 2 min |
| Followed by: | | |
| | 72° C. | 5 min |
| Hold at: | 8° C. | |

DNA fragments of about 800 bp were isolated. The fragments were purified by agarose gel electrophoresis and had A tails added to enable them to be inserted into the CloneTech TA cloning vector (pT-Adv) by incubating the fragment with 2 units Taq Polymerase and 0.2 mM dATP at 72° C. for 10 minutes. Ligation mixtures were set up with a final volume of 10 μl containing 50 ng pT-Adv vector; 50 ng A tailed-PCR product; 1 μl ligase buffer, 10 mM DTT, 1 mM ATP and 0.5 ul T4 DNA ligase. Reactions were incubated at 14° C. for 16 hours. Ligation and transformation was carried out using the AdvanTAge PCR cloning kit from CLONTECH. A 50 ng aliquot of the vector was ligated with the cDNA fragment at 14° C. overnight. Chemically competent TOP10 E. coli cells were transformed with a 2 ul aliquot by heat shock and grown on selected media overnight. A combination of blue/white selection and colony PCR was used to select individual clones containing the pAdvantage vector with inserted cDNA fragments. Individual colonies were grown up and plasmid DNA extracted for sequence analysis.

Sequence Analysis.

The FtsZ DNA fragments present in two independent pT-Adv clones were each sequenced four times in each direction. Analysis of the sequence showed that both of the clones contained a fragment of the FtsZ gene family type designated as type 1. This sequence was designated as potato FtsZ1. The sequence of this fragment is shown in SEQ ID NO: 9.

### Example 4

Isolation of Wheat FtsZ Type 1 cDNA Fragments.

Design of FtsZ type 1 Specific Primers.

Because only type 2 sequences were obtained by PCR using degenerate primers designed using both FtsZ type 1 and FtsZ type 2 sequences, an alternative strategy was employed to obtain a wheat FtsZ type I sequence. PCR primers were designed to the Oryza sativa cDNA sequence. The selected regions corresponded to regions of high homology at the protein level of all the previously listed type 1 sequences and in an equivalent region to the section used for the isolation of the FtsZ type 2 sequences.

### Example 5

Isolation of Full Length Potato FtsZ cDNA Sequences.

Design of specific primers for the isolation of a full length FtsZ type 1 cDNA. The N. tabacum type 1 sequences AJ271749 & AJ133453 were analyzed. Two primers were designed one for each sequence for the 5′ end of the cDNA, designated FZT2FOR and FZT3FOR. A single primer for the 3′ end was designed because both N. tabacum sequences are identical at the 3′ end. Primers were BamHI tailed as described in the Examples above. The sequences of the primers was as follows:

| | |
|---|---|
| FZT2FOR | (SEQ ID NO: 28) |

AGTCGGATCCATGGCCACCATGTTAGGACTCTCAAAC

| | |
|---|---|
| FZT3FOR | (SEQ ID NO: 29) |

AGTCGGATCCATGGCCACCATCTCAAACCCAGCAGAG

| | |
|---|---|
| FZTREV | (SEQ ID NO: 30) |

ACGTGGATCCCTAAAAGAACAGCCTCCGAGTAGGTGT

Design of specific primers for the isolation of a full length FtsZ type 2 cDNA. There was available a Nicotiana tabacum Type 2 sequence (AJ271750). This was analyzed and suitable primers were designed to the 5′ and 3′ ends. The analysis showed the presence of a BamHI restriction enzyme site within the sequence so the primers were tailed with BglII restriction sites (AGATCT). The sequences of the primers is given below:

| | |
|---|---|
| FZTIIFFR | (SEQ ID NO: 31) |

CTGGAGATCTATGGCTACTTGTACATCAGCTGTGTT

| | |
|---|---|
| FZTIIFOR | (SEQ ID NO: 32) |

CTAGAGATCTATGCCTCCTGATACGCGACGGTCACG

| | |
|---|---|
| FZTIIREV | (SEQ ID NO: 33) |

AGTCAGATCTTCTTAAGCTGTTGGGTAGCGTGATCGC

cDNA Synthesis.

mRNA was extracted from leaf and tuber tissue of Solanum tuberosum c.v. Hermes. Double stranded cDNA was synthesized from these mRNA samples using the procedure given in Clontech's SMARTTM PCR cDNA synthesis kit as described for Example 1.

Isolation of Potato FtsZ I Full Length cDNA Fragments.

The cDNA preparations, produced as described above, were used as the template for isolation of a specific cDNA fragment of a potato FtsZ gene by PCR. PCR was carried out using the Advantage 2 PCR kit from CLONTECH The reactions contained 5 ul 10× Advantage Taq buffer; 5 ul 2 mM dNTPs; 0.5 ul of primer FZT3FOR (50 uM); 0.5 ul of primer FZTREV (50 uM); 1 ul cDNA template; 1 ul Advantage Taq polymerase; 37 ul distilled water in a final volume of 50 ul. The PCR was carried out on a thermocycler using the following parameters:

| | | |
|---|---|---|
| Hot start: | 94° C. | 3 min |
| 15 cycles of: | | |
| Step 1 | 94° C. | 1 min |
| Step 2 | 55° C. | 1 min |
| Step 3 | 72° C. | 2 min |
| 15 cycles of: | | |
| Step 1 | 94° C. | 1 min |
| Step 2 | 60° C. | 1 min |
| Step 3 | 72° C. | 2 min |
| Followed by: | | |
| | 72° C. | 5 min |
| Hold at: | 8° C. | |

DNA fragments of about 1500 bp were isolated. The fragments were purified by agarose gel electrophoresis and had A tails added to enable them to be inserted into the CloneTech TA cloning vector (pT-Adv) by incubating the fragment with 2 units Taq Polymerase and 0.2 mM dATP at 72° C. for 10 minutes. Ligation mixtures were set up with a final volume of 10 μl containing 50 ng pT-Adv vector; 50 ng A tailed-PCR product; 1 μl ligase buffer, 10 mM DTT, 1 mM ATP and 0.5 ul T4 DNA ligase. Reactions were incubated at 14° C. for 16 hours. Ligation and transformation was carried out using the AdvanTAge PCR cloning kit from CLONTECH. A 50 ng aliquot of the vector was ligated with the cDNA fragment at 14° C. overnight. Chemically competent TOP10 E. coli cells were transformed with a 2 ul aliquot by heat shock and grown on selected media overnight. A combination of blue/white selection and colony PCR was used to select individual clones containing the pAdvantage vector with inserted cDNA fragments. Individual colonies were grown up and plasmid DNA extracted for sequence analysis.

Isolation of Potato FtsZ 2Full Length cDNA Fragments.

The cDNA preparations, produced as described above, were used as the template for isolation of a specific cDNA fragment of a potato FtsZ gene by PCR. PCR was carried out using the Advantage 2 PCR kit from CLONTECH The reactions contained 5 ul 10× Advantage Taq buffer; 5 ul 2 mM dNTPs; 0.5 ul of primer FZTIIFFR (50 uM); 0.5 ul of primer FZTIIREV (50 uM); 1 ul cDNA template; 1 ul Advantage Taq polymerase; 37 ul distilled water in a final volume of 50 ul. The PCR was carried out on a thermocycler using the following parameters:

| | | |
|---|---|---|
| Hot start: | 94° C. | 3 min |
| 15 cycles of: | | |
| Step 1 | 94° C. | 1 min |
| Step 2 | 55° C. | 1 min |
| Step 3 | 72° C. | 2 min |
| 15 cycles of: | | |
| Step 1 | 94° C. | 1 min |
| Step 2 | 60° C. | 1 min |
| Step 3 | 72° C. | 2 min |
| Followed by: | | |
| | 72° C. | 5 min |
| Hold at: | 8° C. | |

DNA fragments of about 1500 bp were isolated. The fragments were purified by agarose gel electrophoresis and had A tails added to enable them to be inserted into the CloneTech TA cloning vector (pT-Adv) by incubating the fragment with 2 units Taq Polymerase and 0.2 mM dATP at 72° C. for 10 minutes. Ligation mixtures were set up with a final volume of 10 μl containing 50 ng pT-Adv vector; 50 ng A tailed-PCR product; 1 μl ligase buffer, 10 mM DTT, 1 mM ATP and 0.5 ul T4 DNA ligase. Reactions were incubated at 14° C. for 16 hours. Ligation and transformation was carried out using the AdvanTAge PCR cloning kit from CLONTECH. A 50 ng aliquot of the vector was ligated with the cDNA fragment at 14° C. overnight. Chemically competent TOP10 E. coli cells were transformed with a 2 ul aliquot by heat shock and grown on selected media overnight. A combination of blue/white selection and colony PCR was used to select individual clones containing the pAdvantage vector with inserted cDNA fragments. Individual colonies were grown up and plasmid DNA extracted for sequence analysis.

Sequence Analysis.

The FtsZ DNA fragments present in the pT-Adv clones were sequenced four times in each direction. Analysis of the sequence showed that both the FtsZ gene families were represented in the clones. The sequence of these full length cDNA clones is shown in SEQ ID NOS: 11 and 13.

Race

Alternatively full length potato and wheat FtsZ type 1 and FtsZ type 2 cDNA sequences were obtained by 5′ and 3′ RACE.

### Example 6

Construction of Vectors for Potato Transformation

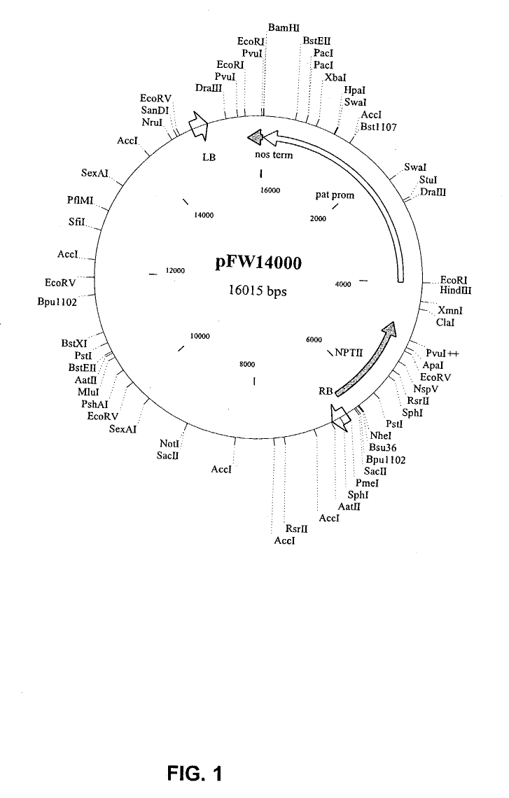

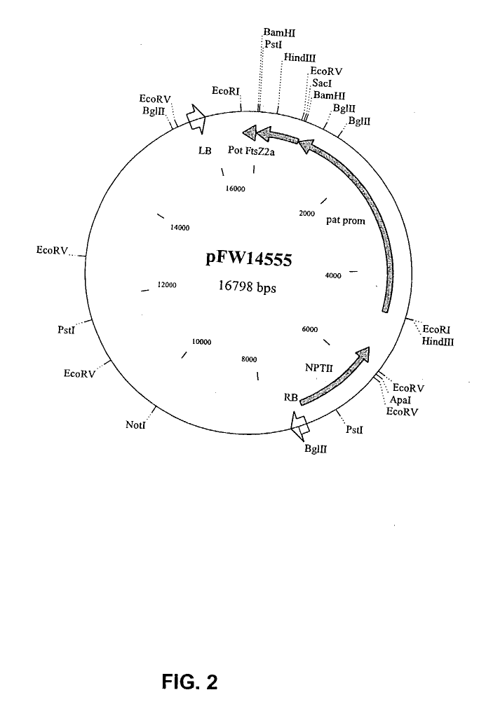

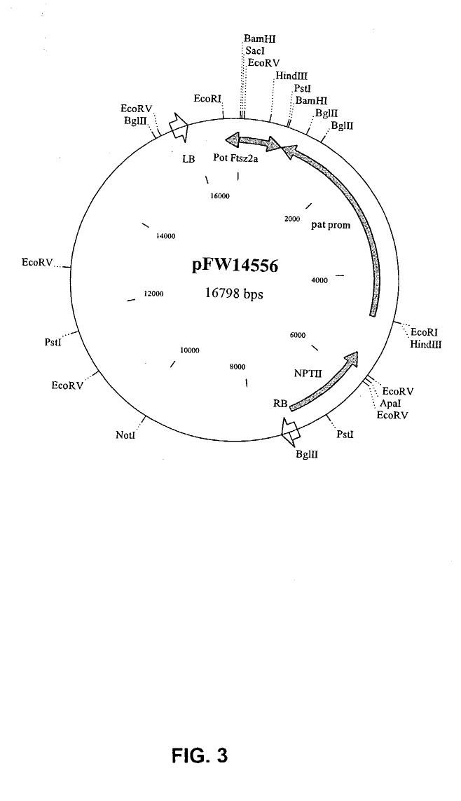

The potato FtsZ2a fragment isolated as described in Example 1 above was cloned into the potato transformation vector pFW14000. The potato transformation vector pFW14000 (FIG. 1) was digested with the restriction enzyme BamHI between the patatin promoter and the nos terminator and dephosphorylated to prevent self ligation. The pT-Adv vector containing the potato FtsZ2a was digested with the restriction enzyme BamHI to release the FtsZ2a fragment. The FtsZ2a fragment was purified by agarose gel electrophoresis. The fragment was ligated into pFW14000 and clones were obtained which had the sequence in either the sense (designated pFW14555, FIG. 2) or antisense (designated pFW14556, FIG. 3) orientations. The transformation vectors so produced were then electroporated into Agrobacterium tumefaciens strain LBA4404 for transformation of potato.

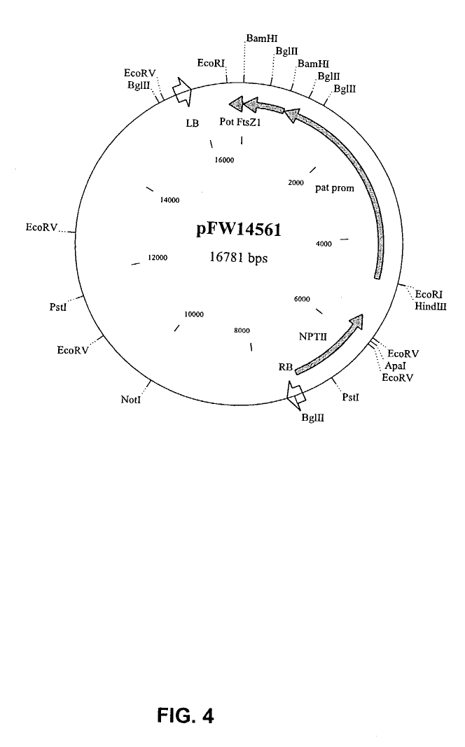

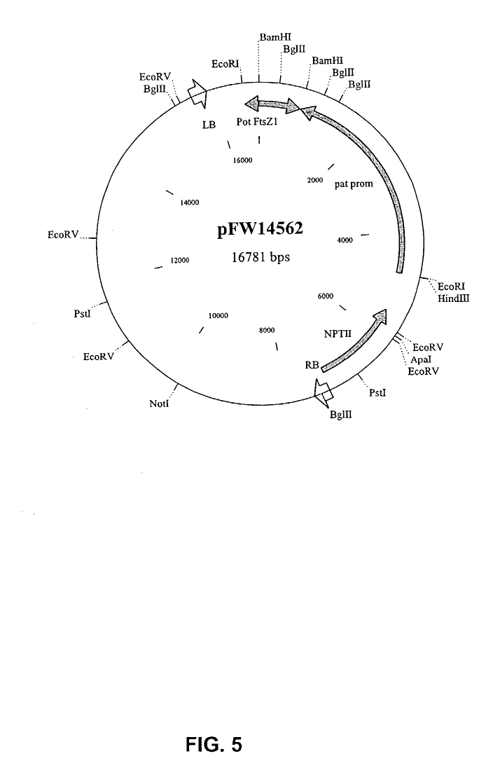

The potato FtsZ1 fragment isolated as described in Example 3 above was cloned into the potato transformation vector pFW14000. The potato transformation vector pFW14000 (FIG. 1) was digested with the restriction enzyme BamHI between the patatin promoter and the nos terminator and dephosphorylated to prevent self ligation. The pT-Adv vector containing the potato FtsZ 1 was digested with the restriction enzyme BamHI to release the FtsZ1 fragment. The FtsZ1 fragment was purified by agarose gel electrophoresis. The fragment was ligated into pFW14000 and clones were obtained which had the sequence in either the sense (designated pFW14561, FIG. 4) or antisense (designated pFW14562, FIG. 5) orientations. The transformation vectors so produced were then electroporated into Agrobacterium tumefaciens strain LBA4404 for transformation of potato.

### Example 7

Construction of Vectors for Wheat Transformation

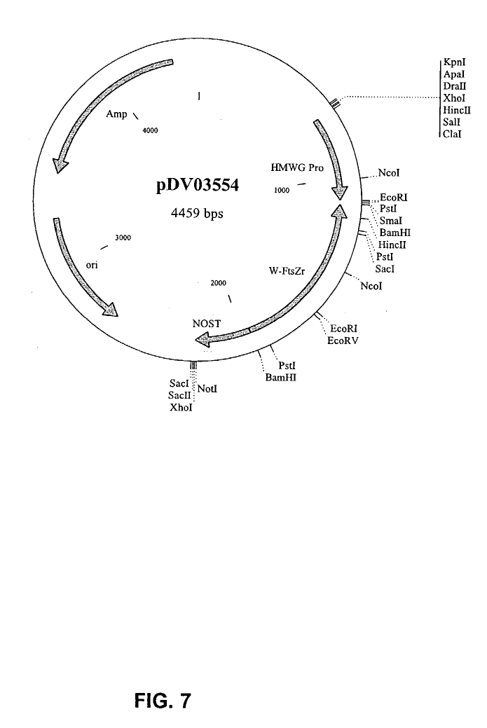

The wheat FtsZ2a fragment, isolated as described in Example 2 was inserted into the vector pDV03000 (WO 00/31274; ATC Ltd.) between the promoter of the high molecular weight glutenin (HMWG) gene (Halford, N. et al. (1989) Plant Science 62 :207-216) and the Nos terminator. A single clone (pT-Adv3-36) containing the wheat FtsZ type II sequence was selected. pAdv3-36 was digested with the restriction enzymes BamHI and ScaI. The ScaI digestion was designed to cut the backbone of the pT-Adv vector so as to prevent it carrying through into the donor vector. The wheat FtsZ2a fragment was purified by agarose gel electrophoresis and ligated into pDV03000 which had been digested with BamHI and dephosphorylated to prevent self-ligation. Ligation mixtures were electroporated into competent E. coli cells and plated out onto selection medium. Resulting colonies were screened by colony PCR and then by restriction enzyme digests to check for the presence of the fragment in the plasmid and to determine its orientation. Clones harboring plasmids having the wheat FtsZ2a fragment present in the sense orientation (designated as pDV03553, FIG. 6) and antisense orientation (designated as pDV03554, FIG. 7) were selected and their sequence verified.

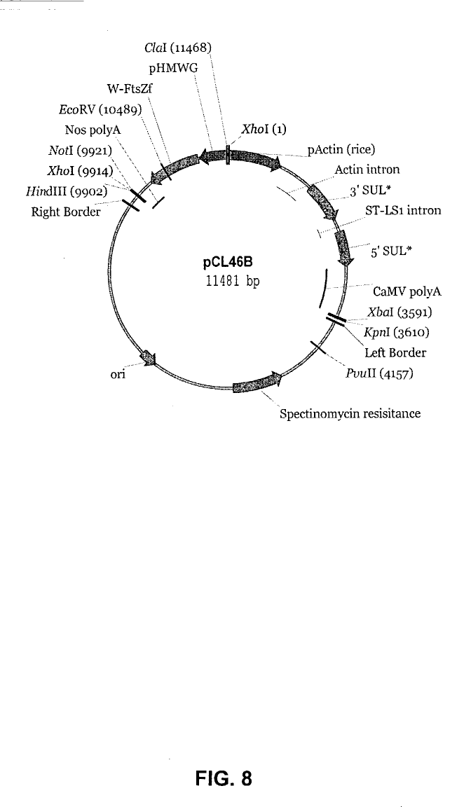

The promoter-coding sequence-terminator cassettes from pDV03553 was inserted into the wheat specific plant transformation binary vector pGB53 as described below. The promoter-coding sequence-terminator cassette of pGB53 based plasmid pGB03205M was excised as a XhoI fragment and replaced by the promoter-coding sequence-terminator XhoI cassette of pDV03553. Competent cells were transformed with the ligation mixture and resulting colonies were screened, one clone was selected and checked using five different restriction digests (PstI, BamHI, EcoRI, NcoI and XhoI). The resulting plasmid is pCL46B (FIG. 8). Plasmid pCL46B was then recombined with pSB1 (Komari et al., Plant J. (1996) 10:165-174) in Agrobacterium tumefaciens strain LBA4404.

### Example 8

Construction of a Vector for Barley Transformation

The pHMWG-senseFtsZ2a cassette from pDV03553 was cloned into a barley specific Agrobacterium vector.

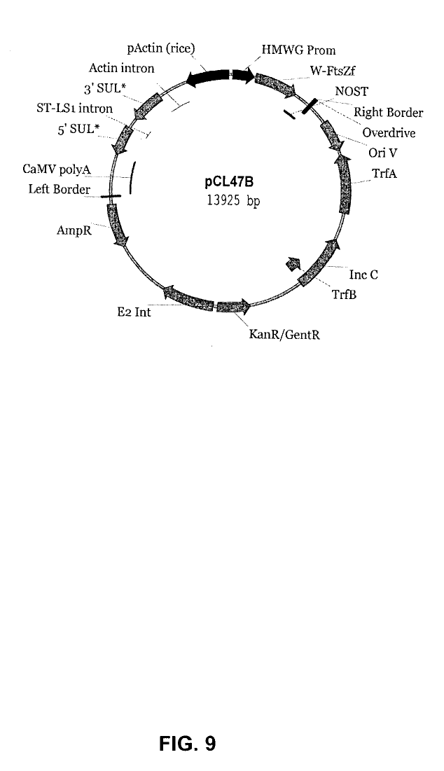

The resulting plasmid, pCL47B, is shown in FIG. 9. The plasmid contains the HMWG promoter driving partial sense FtsZ 2back to back with the Actin promoter driving the selectable marker (sul). The plasmid is in the SCV plant transformation vector, and the Agrobacterium background is Agl1.

### Example 9

Prokaryotic Expression of FtsZ Proteins.

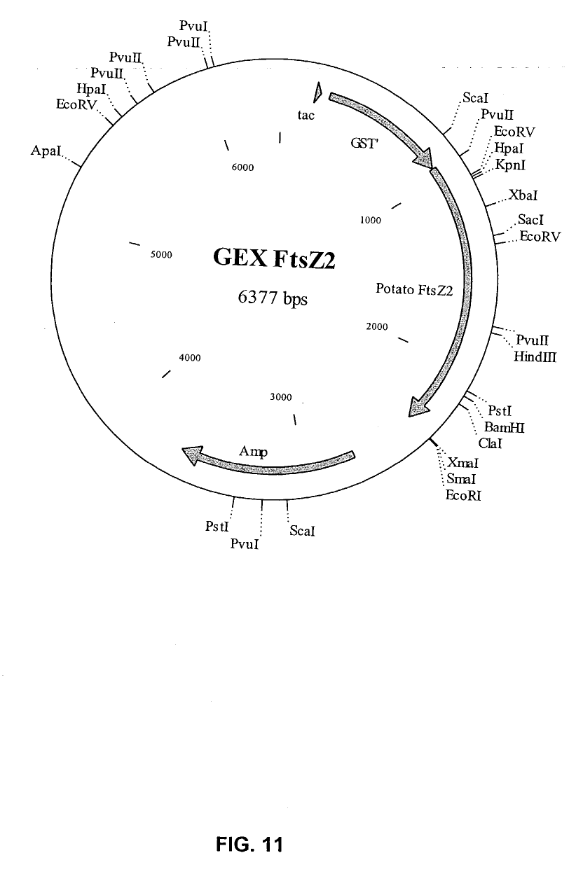

The pT-Adv clones as isolated in Example 5 containing the potato full length DNA fragments for FtsZ type 1 and FtsZ type 2 shown in SEQ ID NOS 11 and 13 were digested with the restriction endonucleases BamHI and BglII respectively and ligated into the E. coli expression vector pGEX2T (Pharmacia) which had also been cut with BamH l restriction endonuclease. This produced the plasmids GEX-FI+(FIG. 10) and GEX-F2+(FIG. 11). The plasmids were electroporated into E. coli XA90 cells. These were plated out onto agar containing kanamycin as a selective agent and grown at 37 C for 16 h. Individual colonies were taken and analyzed for the presence of the FtsZ DNA fragment by PCR. Samples of the cells were grown up at 37° C. in 2% glucose YT medium until an OD600 of 0.6-0.8 was reached. At this stage glutathione-s-transferase-FtsZ fusion protein production was induced in an aliquot of the cells by adding IPTG to a final concentration of 1 mM. These cells were grown on for a further 3 hours at which point they were collected by centrifugation, and whole cell extracts analyzed by SDS-PAGE and compared with cells which had not been induced. There was a novel protein present in the IPTG induced cell extracts of approximately 64 kilodaltons (kDa) which represents the gluthathione-S-transferase-potato FtsZ1 fusion protein.

A pure preparation of the glutathione-S-transferase-potato FtsZ 1 fusion protein was made. E. coli XA90 cells containing the plasmid GEX-FI+ were grown up at 37C in 1 ul of 2% glucose YT medium overnight. This was inoculated into 500 ml of fresh 2% glucose YT medium and grown on at 37° C. until an OD600 of 0.9 was reached. At this point fusion protein production was induced by the addition of IPTG to 1 mM final concentration. The cells were grown for a further 2 hours before they were collected by centrifugation. The cell pellet was resuspended in 50 ml of PBS (50 mM Phosphate buffer, 150 mM NaCl, pH8.0) and sonicated for 2 times 15 seconds. The protein extract was centrifuged at 8000 rpm for 20 minutes at 4° C. and the supernatant decanted into a clean vessel. The fusion protein was purified by affinity chromatography using a GSTrap column (Pharmacia). The clarified supernatant was loaded onto the column and washed with 20 ml of PBS. The bound fusion protein was eluted from the column with 10 ml of 50 mM Tris pH 8.0, 5mM reduced glutathione. Separate 0.5 ml fractions were collected and tested for the presence of fusion protein by SDS-PAGE. A single polypeptide of approximately 64 kDa was isolated from the total soluble proteins. The fractions containing fusion protein (6-9) were pooled and stored.

### Example 10

Transformation of Potato

Solanum tuberosum c.v. Prairie was transformed with pFW14555, pFW14556, pFW14561 and pFW14562 using the method of leaf disk cocultivation essentially as described by Horsch et al. (Science 227: 1229-1231, 1985). The youngest two fully-expanded leaves from a 5-6 week old soil grown potato plant were excised and surface sterilized by immersing the leaves in 8% ‘Domestos’ for 10 minutes. The leaves were then rinsed four times in sterile distilled water. Discs were cut from along the lateral vein of the leaves using a No. 6 cork borer. The discs were placed in a suspension of Agrobacterium, containing one of the four plasmids listed above for approximately 2 minutes. The leaf discs are removed from the suspension, blotted dry and placed on petri dishes (10 leaf discs/plate) containing callusing medium (Murashige and Skoog (MS) agar containing 2.5 ug/ml BAP, 1 ug/ml dimethylaminopurine, 3% (w/v) glucose). After 2 days the discs were transferred onto callusing medium containing 500 μg/ml Claforan and 50 μg/ml Kanamycin. After a further 7 days the discs were transferred (5 leaf discs/plate) to shoot regeneration medium consisting of MS agar containing 2.5 ug/ml BAP, 10 ug/ml GA3, 500 μg/ml Claforan, 50 μg/ml Kanamycin and 3% (w/v) glucose. The discs were transferred to fresh shoot regeneration media every 14 days until shoots appeared. The callus and shoots were excised and placed in liquid MS medium containing 500 μg/ml Claforan and 3% (w/v) glucose. Rooted plants were weaned into soil and grown up under greenhouse conditions to provide tuber material for analysis. Alternatively microtubers were produced by taking nodal pieces of tissue culture grown plants onto MS agar containing 2.5 μg/ml Kanamycin and 6% (w/v) sucrose. These were placed in the dark at 19° C. for 4-6 weeks when microtubers were produced in the leaf axils.

### Example 11

Transformation of Wheat

Spring wheat line NB 1 (Biogemma UK Ltd.) was transformed with Agrobacterium including pCL46B as described in Example 7 using the seed inoculation method described in WO 00/63398 (RhoBio S.A.). Thirteen wheat transformation experiments were initiated in the first instance.

### Example 12

Transformation of Barley

Immature embryos of the barley variety Golden Promise were transformed with pCL47B essentially according to the method of Tingay et al. (The Plant Journal 11 (6) 1369-1376, 1997).

Donor plants of the variety Golden Promise were grown with an 18 h day, and 18/13° C.

Immature embryos (1.5-2.0 mm) were isolated and the axes removed. They were then dipped into an overnight liquid culture of Agrobacterium, blotted and transferred to co-cultivation medium. After 2 days the embryos were transferred to MS based callus induction medium with Asulam and Timentin for 10 days. Tissues were transferred at 2 weekly intervals, and at each transfer they were cut into small pieces and lined out on the plate. At the third transfer, only the embryogenic tissue was moved on to fresh medium. After a total of 8 weeks in culture, the tissue was transferred to regeneration medium (FHG), where plantlets formed within 2-4 weeks. These were transferred to Beatsons glass jar with growth regulator free medium until roots had formed, when they were transferred to Jiffies expandable peat pellets and then to the Conviron growth chamber.

Five Agrobacterium transformation experiments were set up (approximately 600 embryos in total) using the construct pCL47B (FIG. 9).

### Example 13

Analysis of Transformed Plants for Presence of the FtsZ Construct

Analysis of Regenerated Potato Transformants.

Leaf material was taken from regenerated potato plants and genomic DNA isolated. One large potato leaf (approximately 30 mg) was excised from an in vitro grown plant and placed in a 1.5 ml eppendorf tube. The tissue was homogenized using a micropestle and 400 μl extraction buffer (200 mM Tris HCL pH 8.0; 250 mM NaCl; 25 mM EDTA; 0.5% SDS; 40 ug/ml Rnase A) was added and ground again carefully to ensure thorough mixing. Samples were vortex mixed for approximately 5 seconds and then centrifuged at 10,000 rpm for 5 minutes. A 350 μl aliquot of the resulting supernatant was placed in a fresh eppendorf tube and 350 μl chloroform was added. After mixing, the sample was allowed to stand for 5 minutes. This was then centrifuged at 10,000 rpm for 5 minutes. A 300 μl aliquot of the supernatant was removed into a fresh eppendorf tube. To this was added 300 μl of propan-2-ol and mixed by inverting the eppendorf several times. The sample was allowed to stand for 10 minutes. The precipitated DNA was collected by centrifuging at 10,000 rpm for 10 minutes. The supernatant was discarded and the pellet air dried. The pellet of DNA was resuspended in 50 μl of distilled water and was used as a template in PCR. A PCR was then carried out using the primers PS327P and NOS3TP, which are listed below and 1 ul of the plant DNA samples in a 50 ul reaction. A diagnostic DNA fragment of 1015 bp was produced in these reactions, when testing plants transformed with pFW14555 or pFW14556.

| | | |
|---|---|---|
| PS327P | CATCACTAATGACAGTTGCGGTGCAA | (SEQ ID NO: 34) |
| NOS3TP | ATAATCATCGCAAGACCGGCAACAGGA | (SEQ ID NO: 35) |

20 lines of Solanum tuberosum c.v. Prairie pFW14555 and 5 lines of Solanum tuberosum c.v. Prairie pFW14556 were tested and all were shown to contain the construct. The same method was used to analyze Solanum tuberosum c.v. Prairie plants transformed with pFW14561 or pFW14562. In this instance a diagnostic DNA fragment of 1015 bp was produced in these reactions. 37 lines of Solanum tuberosum c.v. Prairie pFW14561 were tested and of these 32 lines were shown to contain the construct. 43 lines of Solanum tuberosum c.v. Prairie pFW14562 were tested and of these 42 lines were shown to contain the construct. The PCR positive plants were selected and used in further experiments.

Analysis of Regenerated Barley Transformants.

A total of 167 plants were recovered and 111 plants were analyzed by PCR for the presence of the introduced FtsZ transgene. Leaf material from plantlets in Jiffy pots, was placed in an Eppendorf tube, frozen in liquid Nitrogen, and ground with a dry plastic drill bit. To this, 400 ul DNA extraction buffer was added and the tubes were left at 65° C. for a minimum of 1 h. The tubes were centrifuged at 13000 rpm for 5 min and the supernatant was added to a tube containing 400 ul iso-propanol, and mixed. After further centrifugation for 5 minutes, the supernatant was discarded and the remaining pellet was resuspended in 50 ul TE buffer and used for the template DNA in the PCR reaction. The primers used were FtsZ for and FtsZrev. A diagnostic fragment of 472 bp is produced.

| | | |
|---|---|---|
| FtsZfor: | GGTGCTCCTGTAATTGCTGG | (SEQ ID NO: 36) |
| FtsZrev: | CATTTCCTCCAGTGATATTCC | (SEQ ID NO: 37) |

PCR reaction mixtures which contained 5 ul 10× Invitrogen Taq buffer; 2.0 ul 50 mM MgCl2 ; 2.5 ul 4 mM dNTPs; 2.5 ul of primer mix FtsZ for (100 mM )and FtsZrev (100 mM); 1.0 ml DNA template (barley genomic DNA or control pCL47B plasmid DNA); 0.25 ul Invitrogen Taq polymerase; 36.75 ul Creosol Red to a final volume of 50 ml were set up. The PCR reaction was carried out in a thermocycler using the following parameters: hot start at 94° C. for 5 min, then 30 cycles of 94° C. for 30 sec, 55° C. for 30 sec 3 min. The cycles were followed by 72° C. for 5 min and the samples held at 24° C.

111 plants were analyzed by PCR and 93 plants were shown to contain the FtsZ transgene. The 93 plants were derived from 46 embryos.

66 plants, derived from 29 embryos, were further characterised by Southern analysis to determine the number of copies of the introduced FtsZ transgene and the number of insertion sites.

Genomic DNA was isolated from Barley leaves using the CTAB extraction method as outlined in: Methods in Molecular Biology vol 28: Protocols for nucleic acid analysis by non-radioactive probes, Isaac P. G. (1994). Humana Press, Totowa, N.J. USA. To determine the number of copies DNA was digested with Bam Hi which releases a single fragment of 828 bp within the Ftsz gene. To determine the number of insertion sites, Xho 1 was used, as this cut once within the T-DNA. The DNA was incubated with the appropriate restriction enzyme overnight at 37° C. The digested DNA was run overnight at 20V out on 0.8% agarose gels. The DNA was then transferred to a nylon membrane by vacuum blotting. The membranes were probed for the FtsZ fragments, at high stringency, and then washed, blocked and labelled with an Anti-Digoxygenin antibody, as described in Methods in Molecular Biology vol 28: Protocols for nucleic acid analysis by non-radioactive probes, Isaac P. G. (1994). Humana Press, Totowa, N.J. USA. The bands were visualized using the CDP-star chemoluminescent spray and then exposed on film.

The Southern analysis showed that the plants derived from a single embryo do not necessarily have the same integration pattern and hence represent different transformation events. The 66 plants analyzed in this way had 36 different integration patterns and therefore represent 36 independent transformation events. The total number of independent transgenic events identified was 65. Plants representing 59 events were fully fertile and have produced mature seed. The PCR and Southern analysis of the transgenic barley plants is presented in Table 1.

TABLE 1
Summary of FtsZ Barley Experiments.

| | | No. of embroys | No. of events | |
|---|---|---|---|---|
| | No. | regenerating | identified by | |
| Expt | embryos | plants confirmed | Southern | No of events |
| No. | plated | by PCR | analysis | producing seed. |
| 1 | 69 | 8 | 20 | 18 |
| 52 | 140 | 2 | 2 | 1 |
| 3 | 225 | 14 | 14 | 13 |
| 4 | 70 | 1 | 1 | 1 |
| 5 | 105 | 21 | 28 | 26 |
| Totals | 609 | 46 | 65 | 59 |

### Example 14

Analysis of Transformed Plants for FtsZ Expression.

Raising Antisera to FtsZ Proteins.

Expression of FtsZ proteins may be analyzed by Western blotting. Antibodies to Ftsz type 1 and FtsZ type 2 were raised by inoculating rabbits with chemically synthesized peptides corresponding to portions of the FtsZ protein sequences, conjugated to keyhole limpet heamocyanin. Diagnostic peptide sequences for the two different proteins have been designed by reference to Stokes et al. (2000) (Plant Physiology 124, 1668-1677), modified according to the specific differences in the Type 2 potato and wheat clones obtained as in Examples 1 and 2 above. The peptide sequences used were:

| | | |
|---|---|---|
| FtsZ1: | EGRKRSLQALEAIE | (SEQ ID NO: 38) |
| FtsZ2: | RRRAVQAQEGIAAL | (SEQ ID NO: 39) |

Preparation of Protein Extracts.

Protein extracts from potato tuber, wheat, barley or maize endosperm were produced by taking up to 100 mg of tissue and homogenizing in 1 ml of ice cold extraction buffer consisting of 50 mM HEPES pH 7.5, 10 mM EDTA, 10 mM DTT. Additionally, protease inhibitors, such as PMSF or pepstatin were included to limit the rate of protein degradation. The extract was centrifuged at 13000 rpm for 1 minute and the supernatant decanted into a fresh eppendorf tube and stored on ice. The supernatants were assayed for soluble protein content using, for example, the BioRad dye-binding protein assay (Bradford, M. C. (1976) Anal. Biochem. 72, 248-254).

An aliquot of the soluble protein sample, containing between 10-50 mg total protein was placed in an eppendorf tube and excess acetone (ca 1.5 ml) added to precipitate the proteins which were collected by centrifuging the sample at 13000 rpm for 5 minutes. The acetone was decanted and the samples air-dried until all the residual acetone has evaporated.

SDS-Polyacrylamide Gel Electrophoresis.

The protein samples were separated by SDS-PAGE. SDS PAGE loading buffer (2% (w/v) SDS; 12% (w/v) glycerol; 50 mM Tris-HCl pH 8.5; 5 mM DTT; 0.01% Serva blue G250) was added to the protein samples (up to 50 ul). Samples were heated at 70° C. for 10 minutes before loading onto a NuPage polyacrylamide gel (Invitrogen). The electrophoresis conditions were 200 V constant for 1 hour on a 10% Bis-Tris precast polyacrylamide gel, using 50 mM MOPS, 50 mM Tris, 1 mM EDTA, 3.5 mM SDS, pH 7.7 running buffer, according to the NuPage methods (Invitrogen, U.S. Pat. No. 5,578,180).

Electroblotting.

Separated proteins were transferred from the acrylamide gel onto PVDF membrane by electroblotting (Transfer buffer: 20% methanol; 25 mM Bicine pH 7.2; 25 mM Bis-Tris, 1 mM EDTA, 50 mM chlorobutanol) in a Novex blotting apparatus at 30 V for 1.5 hours.

Immunodetection.

After blocking the membrane with 5% milk powder in Tris buffered saline (TBS-Tween) (20 mM Tris, pH 7.6; 140 mM NaCl; 0.1% (v/v) Tween-20), the membrane was challenged with a rabbit anti-FtsZ antiserum at a suitable dilution in TBS-Tween. Specific cross-reacting proteins were detected using an anti-rabbit IgG-Horse Radish Peroxidase conjugate secondary antibody and visualized using the enhanced chemiluminescence (ECL) reaction (Amersham Pharmacia).

Antiserum raised to the Type 1 specific peptide was tested for its ability to detect FtsZ proteins from potato tuber, wheat endosperm and maize endosperm. Results showed that the antiserum does cross react with the FtsZ 1 proteins expressed in potato tuber, wheat endosperm and maize endosperm.

rtPCR Analysis.

Expression at the mRNA level was investigated using rtPCR. RNA was extracted from potato tuber tissue using the RNAqueous kit from Ambion. RtPCR was carried out using the reagents and protocols supplied with the RETROscript kit (Ambion). Pairs of primers were designed to detect both the potato FtsZ1 fragment and the potato FtsZ2 fragment as described below.

For potato FtsZ1 fragment:

| | | |
|---|---|---|
| RT561F3 | TCCTCTTTTAGGGGAACAGGCAG | (SEQ ID NO: 40) |
| RT561R3 | CTTCAGCTCGGTTCTTGCTTGATG | (SEQ ID NO: 41) |

For potato FtsZ2 fragment:

| | | |
|---|---|---|
| RT555F1 | TGACAAATTATTGACAGCTGTTTC | (SEQ ID NO: 42) |
| RT555R2 | ACATTAACTAGCCCAGGAATCGTA | (SEQ ID NO: 43) |

These primer sets will amplify both the introduced transgene sequences and the sequences of the endogenous genes. Further sets of primers were designed to sequences only present in the full length endogenous genes, and so will only detect the endogenous gens and not the transgenic fragments as described below. For the potato FtsZI endogenous gene

| | | |
|---|---|---|
| RT563F1 | TGATCCCTCTGCTAACATCATATT | (SEQ ID NO: 44) |
| RT563R1 | ACAGCCTCCGAGTAGGTGTCCGTG | (SEQ ID NO: 45) |

For the potato FtsZ2 endogenous gene

| | | |
|---|---|---|
| RT565F1 | TTGTACATCAGCTGTGTTTATGCC | (SEQ ID NO: 46) |
| RT565R1 | ATCCACCACCTCCTACACCA | (SEQ ID NO: 47) |

Cosupression or antisesne down regulation of the endogenous gene results in a decrease in the transcript levels relative to non transgenic control and this can be observed by semi-quantitative differences upon RT-PCR amplification using the endogenous specific primer sets.

Over-expression of the transgene fragment can increase the template for amplification by the transgene-detecting primers relative to the non transgenic control although the endogenous transcript can be reduced.

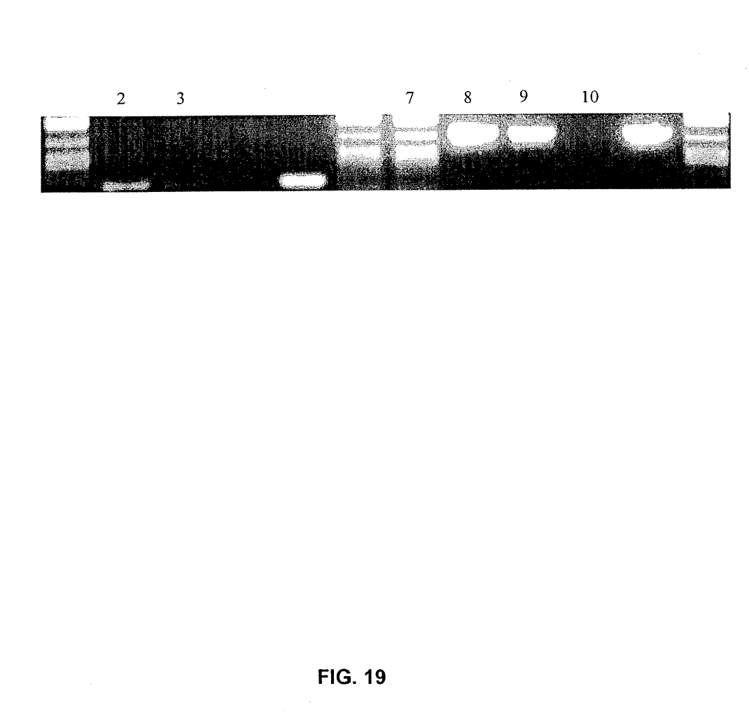

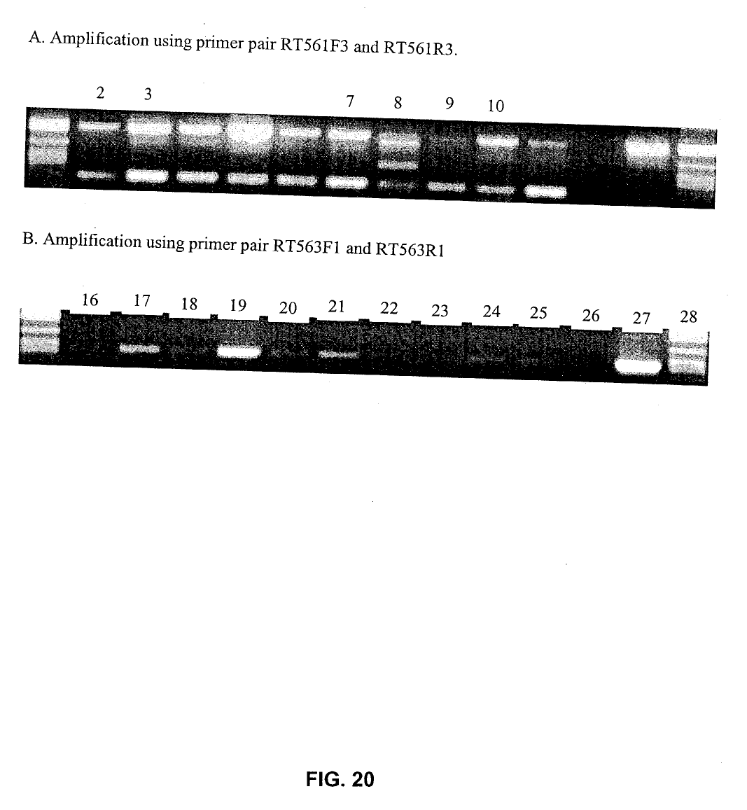

Analyses were performed using the primer combinations designed above using mRNA preparations from tubers of transformed and control non-transformed potato plants. The results of the analysis for plants transformed with pFW14555 are shown in FIG. 19 The results of the analysis for plants transformed with pFW14561 or pFW14562 are shown in FIG. 20. These figures show that the expression patterns of the FtsZ genes were different in the transformed plants compared to controls.

### Example 15

Microscopic Analysis of Amyloplast Size and Number.

Cereal endosperm or potato tuber tissue was fixed, dehydrated and embedded. Samples were taken and sections cut, the sections observed by light microscopy and images captured. The captured images were analyzed for amyloplast numbers per cell and size distribution.

### Example 16

Microscopic Analysis of Starch Granule Size and Number.

Starch granules are extracted from developing and mature cereal endosperm and potato tuber tissues by taking a single endosperm, or 50-100 mg of tuber tissue and homogenizing in 500 ul 1% sodium metabisulphite solution. The starch was collected by centrifugation, 1300 rpm for 5 minutes and then resuspended in 1 ul of water. Aliquots were taken (100 ul) and an equal amount of Lugol solution (Sigma) added to enhance the contrast of the starch granules. Suspensions were prepared for microscope imaging by placing 20 ul onto a graduated microscope slide, covered with a cover slip and sealed with nail varnish. Three representative micrographs were taken of each of the samples and stored electronically. The electronically captured images were then analyzed using suitable image analysis software, such as the package ‘ImageJ’. The raw data was processed to give size range distributions in terms of starch granule diameter classes (measured in micrometer) This enables a quantification of the size distributions of different starch samples to be made and compared. Cumulative frequencies of starch granule size distributions were plotted for each transgenic line and compared with control lines. Statistical significance was determined by using Chi squared tests.

Barley Starch Extraction and Starch Granule Size Measurement from Mature Seeds.

The method described by Zheng and Bhatty (1998, Cereal Chem. 75 p 247-250) was modified for a single kernel extraction. 1 kernel was ground in a ball grinder Retsch (broyeur à bille) in a 10 ml can for 2 minutes at 15 Hz. The ground kernel was suspended in 5 ml of an enzymatic solution (20 mg of Roxazym G : (Roche Vitamins) [Endo-1,4-β-glucanase activity min 8,000 units per gram; Endo-1,3 (4) β-glucanase activity min. 18,000 units per gram; Endo-1,4-β-xylanase activity 26,000 units per gram] in 100 ml of demineralized water).

This suspension was homogenized for 30 seconds with a Vortex agitator and then mixed at room temperature for an additional 30 minutes with constant rotation. The slurry was passed through a 10 μm sieve and washed with demineralized water. The coarse fraction retained by the sieve was discarded. The extract was then passed through a 250 μm sieve. The extract obtained containing starch, protein and β glucan was adjusted to pH 11.5 with 0.1 M NaOH, stirred for 15 minutes at room temperature and then centrifuged at 3000 g for 5 minutes.

The supernatant was discarded and the starch was re-suspended in demineralized water and re-centrifuged as describe above. This procedure was repeated once. The pooled starch was suspended in 95% ethanol and centrifuged at 3000 g for 5 minutes. The supernatant was discarded and pooled starch re-suspended in 95% ethanol. The slurry was then screened through a 67 μm sieve washed with 95% ethanol. The extract was suspended in 95% ethanol, centrifuged at 3000 g for 5 minutes and the pooled starch suspended in 1 ml of 95% ethanol and immediately analyzed with a laser particle size instrument (Malvern) with a 45 mm focal length (0.1 to 80 μm size range measurement) in ethanol.

The calculation of small granules (B) and large granules (A) mean sizes and percentages was managed with the Mastersizer software. Results for 4 transgenic barley lines and 3 non-transformed controls are shown in the Table 2 below, which shows that the mean A and B starch granule sizes of the transformed plants are both lower than those of the controls and that the relative proportions of the A and B granules are different in the transformants compared to the controls.

TABLE 2
Mean starch granule size and distributions from mature barley seed.

| | Mean B | | Mean A | | | | | |
|---|---|---|---|---|---|---|---|---|
| | (μM) | granule size | (μm) | granule size | % B granules | | % A granules | |
| | mean | SEM | mean | SEM | mean | SEM | mean | SEM |
| Transgenic | 2.73 | 0.03 | 15.90 | 0.00 | 12.75 | 0.75 | 74.75 | 1.49 |
| Controls | 2.92 | 0.07 | 17.12 | 0.46 | 13 | 0.58 | 75.5 | 1.38 |

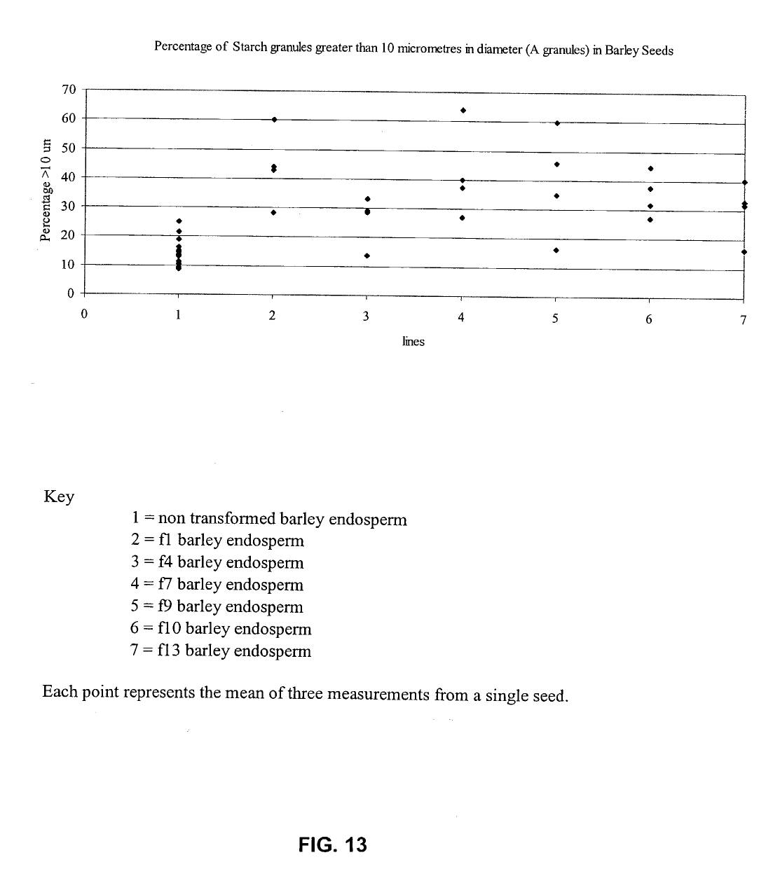

Starch samples were obtained from the endosperm of 22 barley lines transformed with pCL47B as described above in Example 12. These were analyzed microscopically as described. The data for the control and transgenic lines were plotted in two ways. The frequency plots (FIG. 12) show that there are two main size classes, which corresponds to what is known for barley starch. A cumulative frequency plot allowed the distributions between different samples to be compared statistically using a Chi squared test. FIG. 12 shows that the starch granule distributions of 6 transgenic lines are significantly different from the control starch granule distributions, as shown by a Chi squared test for significance. For the lines analyzed, the percentage of granules over 10 mm for each seed of each line and control was calculated and shown in FIG. 13 which clearly shows that the A/B granule ratio in the barley transformed seed is different to that in the control lines. When analyzed microscopically, the starch from the endosperm of one transformed barley line, f58, contained unusually large starch granules.

Analysis of Size Distributions of Starch Granules from Potato Micotubers.

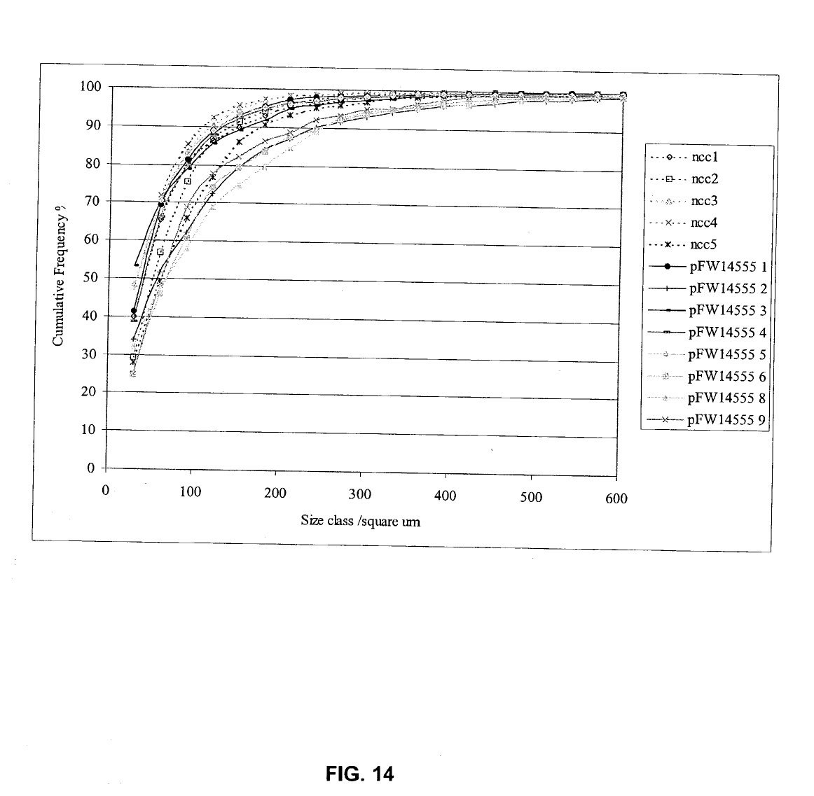

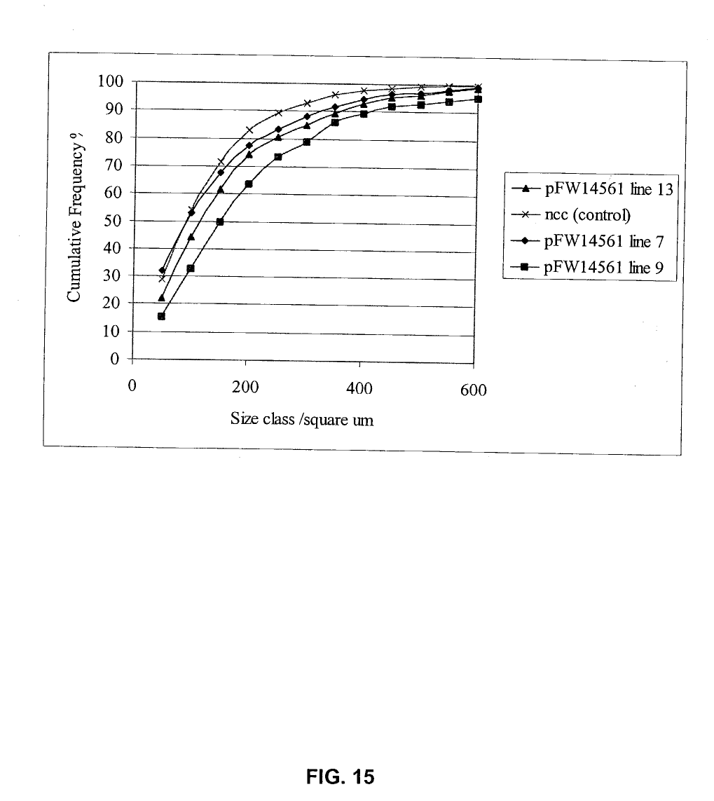

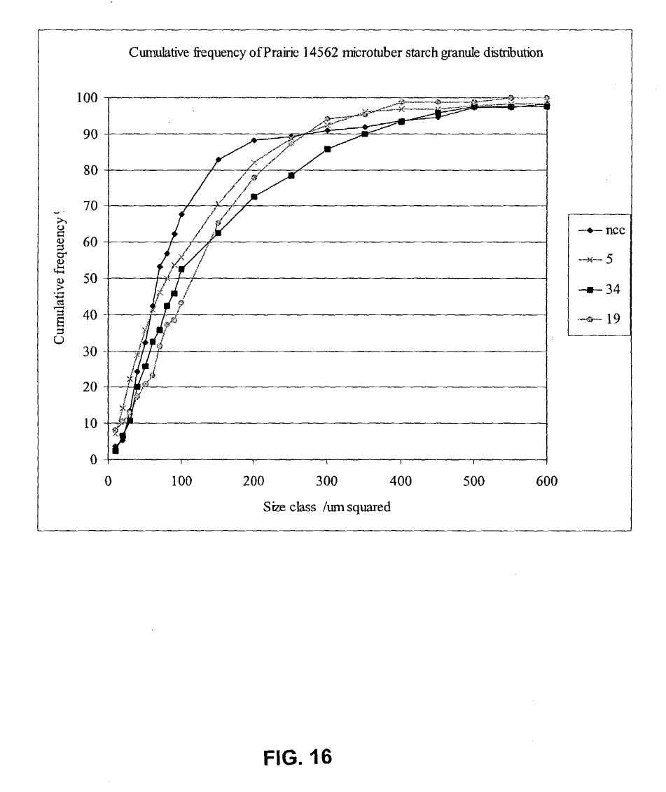

Starch samples were obtained from microtubers of Solanum tuberosum c.v. Prairie lines transformed with the constructs pFW14555, pFW14556, pFW14561 or pFW14562 as described above. These were analyzed microscopically as described. The processed results are shown as cumulative frequency plots in FIGS. 14, 15 and 16. These graphs show that the starch granule distributions of lines pFW14555 2, 6, 8, 9; pFW14561 4, 9, 11, 13, 16, 19, 22, 31; pFW14562 4, 5, 14, 19, 23, 28, 34 and 38 are significantly different from the control starch granule distributions, as demonstrated by a Chi squared test for significance.

Analysis of Size Distributions of Starch Granules from Potato Tubers.

Lines were selected to be grown up to full sized tubers on the basis of the microtuber data shown above. Starch samples were obtained from tubers of 21 Solanum tuberosum c.v. Prairie lines transformed with the constructs described in Example 6 which had been grown in a greenhouse. These were analyzed microscopically as described above. The processed results are shown as cumulative frequency plots in FIG. 17. The starch granule distributions of lines pFW14555 line 2; pFW14561 lines 4, 13, 16; pFW14562 lines 5, 23, 28, 34 and 56 are significantly different from the control starch granule distributions, as demonstrated by a Chi squared test for significance. pFW14555 line 2 exhibited a decrease in the height of the peak of the distribution of starch granule sizes, i.e. a more uniform distribution of sizes of starch granules in comparison to non-engineered control plants. pFW14561 lines 4, 13, 16 exhibited a decrease in the height of the peak of the distribution of starch granule sizes, i.e. a more uniform distribution of sizes of starch granules in comparison to non-engineered control plants. pFW14562 lines 5, 19, 23, 28, 34 and 56 exhibited both a decrease in the height of the peak of the distribution of starch granule sizes, and a shift in the peak towards larger size granules in comparison to non-engineered control plants.

### Example 17

Analysis of Starch Functionality.

Preparation of Starch from Cereal Endosperm.

Starch was extracted from grain of separate wheat and barley lines. Samples (3-4 g) were placed in a mortar, 30 ml of 1% Sodium bisulphite added and placed on ice for 30 minutes. The grains were then gently pulverized using a pestle. The solution was filtered through a nylon filter sieve and collected in a centrifuge tube. The pulverized wheat was re-extracted with a further 30 ml of 1% Sodium bisulphite, the filtrates combined and centrifuged at 6000 rpm for 5 minutes. After decanting off the supernatant, the starch pellet was resuspended in water and centrifuged at 6000 rpm for 5 minutes. This was repeated once. The resulting starch pellet was resuspended in acetone, centrifuged at 6000 rpm for 5 minutes and the supernatant decanted away. This was repeated once and the starch left to air dry. Once dried the starch was stored at −20° C.

Preparation of Starch from Potato Tubers.

Starch was extracted from potato tubers by taking 0.5-1 kg of washed tuber tissue and homogenizing using a juicerator (Waring) chased with 200 ml of 1% Sodium bisulphite solution. The starch was allowed to settle, the supernatant decanted off and the starch washed by resuspending in 200 ml of ice-cold water. The resulting starch pellet was resuspended in acetone and the starch left to air dry. Once dried the starch was stored at −20° C.

Viscometric Analysis of Starch.

Starch samples were analyzed for functionality by testing rheological properties using viscometric analysis. Potato tuber starch from greenhouse grown tubers was analyzed by Differential Scanning Calorimetry (DSC). DSC is a measure of the gelatinisation behavior of starch. The results are shown in FIG. 18. The range of delta H (DH) values of the control samples was 13.3-15.2 J/g. Several of the starch samples from the transformed plants have values which lie outside of this range, including 14555-8, at 15.4 J/g, which may require more energy to form a gel than starch samples from non-transformed plants and 14561-9 at 12.7 J/g; 14561-16 at 13.2 J/g; 14562-23 at 13.0 J/g; and 14562-34 at J/g. which may require less energy to form gels than starch samples from non-transformed plants.

What is claimed is:

1. An isolated nucleic acid molecule that:
(i) comprises a nucleotide sequence that encodes a polypeptide comprising an amino acid sequence that is at least 98% identical to SEQ ID NO: 2, 4, 6, 8, or 10, or a fragment thereof;
(ii) comprises a nucleotide sequence that is at least 94% identical to SEQ ID NOs: 1, 3, 5, 7, or 9, or a complement thereof; or
(iii) hybridizes to a nucleic acid molecule consisting of SEQ ID NO: 1, 3, 5, 7, or 9, or a complement thereof, under conditions of hybridization comprising washing at 60° C. twice for 15 minutes in 2×SSC, 0.5% SDS.

2. An isolated nucleic acid molecule that:
(i) comprises a nucleotide sequence that encodes a polypeptide comprising an amino acid sequence that is at least 93% identical to SEQ ID NO: 12 or 14, or a fragment thereof;
(ii) comprises a nucleotide sequence that is at least 92% identical to SEQ ID NO: 11 or 13, or a complement thereof; or
(iii) hybridizes to a nucleic acid molecule consisting of SEQ ID NO: 11 or 13, or a complement thereof, under conditions of hybridization comprising washing at 60° C. twice for 15 minutes in 2×SSC, 0.5% SDS.

3. An isolated nucleic acid molecule that:
(i) comprises a nucleotide sequence that encodes a polypeptide comprising an amino acid sequence that is at least 95% identical to SEQ ID NO: 16, 18, or 20, or a fragment thereof;
(ii) comprises a nucleotide sequence that is at least 90% identical to SEQ ID NO: 15, 17, 19, or 21, or a complement thereof; or
(iii) hybridizes to a nucleic acid molecule consisting of SEQ ID NO: 15, 17, 19, or 21, or a complement thereof, under conditions of hybridization comprising washing at 60° C. twice for 15 minutes in 2×SSC, 0.5% SDS.

4. A fragment of the isolated nucleic acid molecule of claims 1, 2, or 3, wherein the fragment comprises at least 40, 60, 80, 100 or 150 contiguous nucleotides of the nucleic acid molecule.

5. An isolated polypeptide comprising:
(i) an amino acid sequence that is at least 98% identical to SEQ ID NO: 2, 4, 6, 8, or 10, or an at least 8, 10, 15, 20, 25, 30 or 35 amino acid fragment thereof;
(ii) an amino acid sequence encoded by the nucleic acid molecule of claim 1; or
(iii) an amino acid sequence of SEQ ID NO: 2, 4, 6, 8, or 10, or an at least 8, 10, 15, 20, 25, 30 or 35 amino acid fragment thereof.

6. An isolated polypeptide comprising:
(i) an amino acid sequence that is at least 93% identical to SEQ ID NO: 12 or 14, or an at least 8, 10, 15, 20, 25, 30 or 35 amino acid fragment thereof;
(ii) an amino acid sequence encoded by the nucleic acid molecule of claim 2; or
(iii) an amino acid sequence of SEQ ID NO: 11 or 13, or an at least 8, 10, 15, 20, 25, 30 or 35 amino acid fragment thereof.

7. An isolated polypeptide comprising:
(i) an amino acid sequence that is at least 95% identical to SEQ ID NO: 16, 18, or 20, or an at least 8, 10, 15, 20, 25, 30 or 35 amino acid fragment thereof;
(iii) an amino acid sequence encoded by the nucleic acid molecule of claim 3; or
(v) an amino acid sequence of SEQ ID NO: 16, 18, or 20, or an at least 8, 10, 15, 20, 25, 30 or 35 amino acid fragment thereof..

8. A polypeptide comprising an amino acid sequence of SEQ ID NO: 2, 4, 6, 8, 10, 12, 14, 16, 18, or 20 with one or more conservative amino acid substitutions.

9. A fusion polypeptide comprising the amino acid sequence of any one of claims 5, 6, or 7 and a heterologous polypeptide.

10. A fragment or immunogenic fragment of the polypeptide of any one of claims 5, 6, or 7, wherein the fragment comprises at least 8, 10, 15, 20, 25, 30 or 35 consecutive amino acids of the polypeptide.

11. A method for making the polypeptide of any one of the claims 5, 6, or 7, comprising the steps of:
(a) culturing a cell comprising a recombinant polynucleotide encoding the polypeptide, under conditions that allow said polypeptide to be expressed by said cell; and
(b) recovering the expressed polypeptide.

12. A vector comprising the nucleic acid molecule of any one of claims 1, 2, or 3.

13. An expression vector comprising the nucleic acid molecule of any one of claims 1, 2, or 3 and at least one regulatory region operably linked to the nucleic acid molecule.

14. The expression vector of claim 13, wherein the regulatory region confers chemically-inducible, dark-inducible, developmentally regulated, developmental-stage specific, wound-induced, environmental factor-regulated, organ-specific, cell-specific, and/or tissue-specific expression of the nucleic acid molecule, or constitutive expression of the nucleic acid molecule.

15. The expression vector of claim 13, wherein the regulatory region is selected from the group consisting of a 35S CaMV promoter, a rice actin promoter, a patatin promoter, and a high molecular weight glutenin gene of wheat.

16. An expression vector comprising the antisense nucleotide sequence of the nucleic acid molecule of any one of claims 1, 2, or 3, wherein the antisense sequence is operably linked to at least one regulatory region.

17. A genetically-engineered cell which comprises the nucleic acid molecule of any one of claims 1, 2, or 3 operably linked to a heterologous regulatory region.

18. A cell comprising the expression vector of claim 13.

19. A cell comprising the expression vector of claim 18.

20. A genetically-engineered plant or progeny thereof comprising the nucleic acid molecule of any one of claims 1, 2, or 3 operably linked to a heterologous regulatory region.

21. The plant of claim 20, wherein the nucleic acid molecule comprises an antisense nucleotide sequence.

22. A plant part comprising the nucleic acid molecule of any one of claims 1, 2, or 3 operably linked to a heterologous regulatory region, wherein the overall size of starch granules is altered relative to a plant part not comprising the nucleic acid molecule.

23. The plant part of claim 22, wherein the part is a tuber, stem, root, seed, or seed endosperm.

24. Altered starch obtained from the plant of claim 20.

25. Altered starch obtained from the plant of claim 21.

26. Starch granules obtained from the plant of claim 20, wherein at least one of the starch granules is larger than any of the granules found in a plant without the nucleic acid molecule.

27. Starch granules obtained from the plant of claim 21, wherein the starch granules are larger than any found in the plant without the nucleic acid molecule.

28. A method of altering the sizes of starch granules comprising introducing into a first plant an expression vector of claim 13, and growing the first plant such that the nucleic acid molecule in the expression vector is expressed, wherein the size of the starch granules is altered relative to a second plant that does not contain the expression vector.

29. The method of claim 28, wherein the size of one or more starch granule is larger than any found in the second plant.

30. The method of claim 28, wherein altering the sizes of starch granules results in an increase in a ratio of large to small starch granules.

31. The method of claim 28, wherein altering the sizes of starch granules results in an decrease in a ratio of large to small starch granules.

32. The method of claim 30 or 31, wherein the small starch granules are less than or equal to 10 um in diameter and the large starch granules are greater than 10 um in diameter.

33. The method of claim 28, wherein altering the sizes of starch granules results in a shift in a distribution of starch granule size towards larger granules.

34. The method of claim 28, wherein altering the sizes of starch granules results in a shift in a distribution of starch granule size towards smaller granules.

35. The method of claim 28, wherein altering the sizes of starch granules results in a shift in a distribution of starch granule size, wherein a peak in the distribution widens.

36. A method of making starch granules comprising,
a) growing a plant comprising a nucleic acid of any one of claims 1, 2, or 3 operably linked to a heterologous regulatory region, such that the overall size of the starch granules is altered relative to that of a plant without the nucleic acid; and
b) extracting the starch granules from the plant.

37. A method of altering one or more starch characteristics comprising growing a plant comprising a nucleic acid of any one of claims 1, 2, or 3 operably linked to a heterologous regulatory region, such that the overall size of the starch granules is altered relative to that of a plant without the nucleic acid, wherein the characteristics of the starch from the plant with the nucleic acid is modified relative to a plant without the nucleic acid.

38. The method of claim 36, wherein the characteristic altered is selected from the group consisting of viscosity, gelling, thickness, foam density, or pasting.

39. A method for altering starch granule quantity comprising, introducing into a plant an expression vector of claim 13, such that the quantity of starch granules is altered relative to a plant without the expression vector.

40. A genetically-engineered potato cell comprising a patatin promoter operably linked to a nucleic acid molecule of SEQ ID NO: 1, such that said patatin promoter regulates transcription of said molecule, and wherein sizes of starch granules in the cell are altered relative to a potato cell not comprising the nucleic acid molecule.

41. A genetically-engineered potato cell comprising a patatin promoter operably linked to a nucleic acid molecule of SEQ ID NO: 9 in an antisense orientation, such that said patatin promoter regulates transcription of said molecule, and wherein sizes of starch granules in the cell are altered relative to a potato cell not comprising the nucleic acid molecule.

42. A genetically-engineered cereal cell comprising a HMWG promoter operably linked to a nucleic acid molecule of SEQ ID NO: 5 in an antisense orientation, such that said HMWG promoter regulates transcription of said molecule, and wherein sizes of starch granules in the cell exhibit an increase in a ratio of large to small granules relative to a cereal cell not comprising the nucleic acid molecule.

43. A plant derived from the genetically-engineered cell of any one of claims 40, 41 or 42.

44. Altered starch extracted from a plant of claim 43.

45. The altered starch of claim 44, comprising starch granules of a more uniform size.
